(19) United States
(12) Patent Application Publication
(10) Pub. No.: US 2012/0148594 A1
(43) Pub. Date: Jun. 14, 2012

(54) MIMOTOPES OF HIV AND USES THEREOF

(75) Inventors: Michael Humbert, Boston, MA (US); Ruth M. Ruprecht, Brookline, MA (US); Anton M. Sholukh, Allston, MA (US)

(73) Assignee: DANA-FARBER CANCER INSTITUTE, INC., Boston, MA (US)

(21) Appl. No.: 13/391,498

(22) PCT Filed: Aug. 23, 2010
(86) PCT No.: PCT/US10/46353

§ 371 Date: Feb. 21, 2012

Related U.S. Application Data
(60) Provisional application No. 61/235,942, filed on Aug. 21, 2009.
(60) Provisional application No. 61/351,637, filed on Jun. 4, 2010.

(51) Int. Cl. A61K 39/395; A61K 39/12; A61K 39/02; A61K 39/002; C12Q 1/02; C12N 5/10; C12N 5/00; C12N 1/21; C12N 1/19; C12N 15/63; A61K 38/00; A61K 31/7088; C07K 7/08; C07K 7/06; C07K 16/00; C07H 21/04; A61P 33/00; A61P 33/02; A61P 31/04; A61P 31/10; A61P 37/04; A61P 31/12; A61K 39/00

(52) U.S. Cl. 424/139.1; 424/184.1; 424/185.1; 424/204.1; 424/234.1; 424/265.1; 424/274.1; 435/29; 435/325; 435/348; 435/252.3; 435/252.31; 435/252.33; 435/252.35; 435/254.21; 435/320.1; 514/1.1; 514/44 R; 530/327; 530/329; 530/387.9; 530/388.1; 536/23.1

(57) ABSTRACT

The invention provides methods, compositions and kits for treating and or preventing diseases having immunological components associated with their etiology and/or progression (e.g., an HIV infection). A mimotope that mimics an epitope of an antigen may be administered to an individual to induce or enhance an immune response for the treatment of a disease. For example, HIV envelope-like polypeptides (wild-type HIV polypeptides and mimotopes) may be administered to an individual so as to induce a protective immune response to HIV. Alternatively, antibodies directed to a mimotope may be administered to an individual to treat or prevent a disease. Antibodies directed to HIV envelope-like polypeptides may be administered to an individual to treat or prevent an HIV infection and/or one or more symptoms associated with the infection (e.g., AIDS). In another embodiment, the invention provides compositions and methods for isolating an epitope specific B cell.

### CROSS-REFERENCE TO RELATED APPLICATION

This application claims the benefit of the following U.S. Provisional Application Ser. No. 61/235,942, filed Aug. 21, 2009, and Ser. No. 61/351,637, filed Jun. 4, 2010; the entire contents of each of which are incorporated herein by this reference.

### STATEMENT OF RIGHTS TO INVENTIONS MADE UNDER FEDERALLY SPONSORED RESEARCH

This work was supported by the following grants from the National Institutes of Health, Grant No: P01 AI048240. The government has certain rights in the invention.

### BACKGROUND OF THE INVENTION

The immune system defends the body against infection and a wide variety of other diseases. Methods for enhancing the body's ability to fight disease have been widely used for the treatment of infectious diseases. Recent developments indicate that immune modulation can be used to treat virtually any disease having immunological components associated with its etiology and/or progression. Recent pre-clinical and clinical studies have suggested that active or passive immunization may be useful not only for infectious diseases, such as HIV, but also for the treatment of cancer, Alzheimer's disease, artherosclerotic cardiovascular disease, inflammation, or autoimmune disease. Passive immunization involves the administration of monoclonal or polyclonal antibodies to an individual to prevent or treat infections or disease. To identify promising vaccine candidates for active immunization for the prevention or treatment of such diseases, it is important to identify those portions of an antigen that are immunogenic and that induce protective antibodies in the host. When such information is lacking, vaccine development can be challenging.

Acquired immune deficiency syndrome (AIDS) is a major health threat throughout the world. HIV is the leading cause of AIDS. To date there are no effective vaccines for AIDS. One target of therapeutic vaccines is the envelope protein of HIV (gp160). During infection, gp160 is cleaved by host cell proteases to from the gp120 and gp41. To identify promising vaccine candidates directed to HIV-1 Env, X-ray crystallography has revealed important structural Env features and sites of interaction with cellular receptors. Nevertheless, no effective HIV vaccine is currently available. Given the devastating effects of the illness throughout the world, vaccines to prevent or treat global diseases, such as HIV, are urgently required.

### SUMMARY OF THE INVENTION

The invention provides methods, compositions and kits for treating and or preventing diseases having immunological components associated with their etiology and/or progression. For example, a mimotope that mimics a specific domain of an antigen may be administered to an individual to induce or enhance an immune response for the treatment of a disease. Alternatively, antibodies directed to the mimotope may be administered to an individual to treat or prevent the disease (e.g., an infectious disease, such as HIV, cancer, Alzheimer's disease, artherosclerotic cardiovascular disease, inflammation, or autoimmune disease) and/or one or more symptoms associated with the disease. In other embodiments, the invention provides compositions and methods for isolating epitope-specific B cells using a fusion protein comprising a detectably labelled mimotope. This allows the isolation of B-cells with desired epitope specificity.

In one aspect, the invention is directed to a method of inducing neutralizing antibodies in a subject by administering an expression vector encoding an antigen or entire antigen in an amount insufficient to induce an antibody response in the subject and administering an effective amount of a polypeptide containing a mimotope of a specific subdomain of the antigen, where the method induces a neutralizing antibody response in the subject. In various embodiments, the antigen is derived from a pathogen, including one or more of a bacteria, virus, fungus, protozoan, or parasite. In various embodiments, the antigen is derived from an autoimmune disease. In some embodiments, the expression vector is a DNA vector. Antibodies produced according to methods of the invention may be used therapeutic, prophylactic, or diagnostic purposes

In another aspect, the invention provides a method for isolating an antibody expressing B cell that specifically binds an epitope or a mimotope (i.e., a discrete epitope of an antigen), the method involving contacting the B cell with a detectable fusion polypeptide containing an immunogenic polypeptide fragment thereof (“mimotope”), thereby labeling the B cell, and isolating a B cell that specifically binds to the epitope or mimotope. The invention is directed to isolating an individual B cell expressing an antibody with any desired epitope specificity, as dictated by use of a fluorescently labeled mimotope. In various embodiments, the detectable fusion polypeptide contains a reporter selected from the group consisting of GFP, YFP, BFP, and CFP. In specific embodiments, the reporter is GFP. In various embodiments, the cells are isolated using flow cytometry, affinity purification, affinity chromatography, or column chromatography. In further various embodiments, the method involves amplifying the polynucleotide encoding the V_{H} or V_{L} regions from the B cell. 10. In other embodiments, the method further involves cloning the amplified polynucleotide into a vector encoding an immunoglobulin scaffold, and using the vector to generate a recombinant antibody or fragment thereof, thereby generating an epitope-specific monoclonal antibody, or a fragment thereof. In some embodiments, the method further involves administering the antibodies to a subject for passive immunization. Monoclonal antibodies produced according to methods of the invention may be used therapeutic, prophylactic, or diagnostic purposes.

In yet another aspect, the invention provides a method of treating or preventing a disease in a subject, the method involving administering a polypeptide containing a mimotope of an antigen to induce or enhance an immune response in the subject, thereby treating the disease in the subject. In various embodiments, the disease is a bacterial infection, a viral infection, a fungal infection, a protozoan infection, or autoimmune disease.

The invention provides methods, compositions and kits for treating and or preventing an HIV infection. For example, HIV envelope-like polypeptides (wild-type HIV polypeptides and mimotopes) may be administered to an individual so as to induce a protective immune response to HIV. Alternatively, antibodies directed to the HIV envelope-like polypeptides may be administered to an individual to treat or prevent an HIV infection and/or one or more symptoms associated with the infection (e.g., AIDS). In other embodiments, the invention provides compositions and methods for isolating HIV epitope-specific B cells using a fusion protein comprising a detectably labelled mimotope (e.g., linked to a fluorescent protein).

The present invention is based, at least in part, by the identification of neutralizing antibodies from rhesus macaques infected with SHIV-1157ip (a chimera that encodes env of a recently transmitted HIV-C strain) or the related SHIV-1157ipd3N4 (Song R J, et al. (2006). J Virol 80: 8729-8738). These macaques developed high-titer neutralizing antibody (nAb) responses against homologous SHIV-clade C as well as heterologous primary strains of HIV-1 from different clades. These neutralizing antibodies were employed in phage display to identify the HIV-C Env structures (mimotopes and wild-type polypeptides) recognized by the broadly reactive antibodies.

In one aspect, the invention is directed to an isolated HIV envelope-like polypeptide. Generally, the HIV envelope-like polypeptides will have an amino acid sequence which is 80% or more identical to an amino acid sequence depicted in FIG. 1 (SEQ ID NOs 1-84), FIG. 16, or provided at SEQ ID NOs 94-109. Suitable HIV envelope-like polypeptides include both mimotope and wild-type HIV envelope polypeptides (gp160).

The invention is also directed to nucleic acids encoding the HIV envelope-like polypeptides. The nucleic acids may be isolated, in a vector or expressed in a host cell.

The invention is also directed to antibodies that specifically bind to HIV envelope-like polypeptides. Generally, the antibodies are directed against HIV envelope-like polypeptides having an amino acid sequence which is 80% or more identical to an amino acid sequence depicted in FIG. 1 (SEQ ID NOs 1-84), FIG. 16, or provided at SEQ ID NOs 94-109. Suitable HIV envelope-like polypeptides include both mimotope and wild-type polypeptides. The antibody may be derived from a vertebrate, preferably a mouse, human or monkey and can be either polyclonal or monoclonal. Chimeric and humanized antibodies as well as antibody binding fragments are also contemplated.

Any of the above HIV envelope-like polypeptides, nucleic acids and antibodies may be present in a kit or composition, e.g., pharmaceutical composition.

In yet another embodiment, the invention is directed to a method for inducing an immune response in a subject by administering a pharmaceutical composition having an immunologically effective dose of an HIV envelope-like polypeptide. Generally, the HIV envelope-like polypeptide is 80% or more identical to an amino acid sequence illustrated in FIG. 1 (SEQ ID NOs 1-84), FIG. 16, or provided at SEQ ID NOs 94-109. Suitable HIV envelope-like polypeptides include both mimotope and wild-type HIV envelope polypeptides. In some embodiments, the method further includes the sequential or co-administration of a nucleic acid encoding a HIV envelope polypeptide (e.g., gp160).

Administering a therapeutically effective dose of an antibody that specifically binds to an HIV envelope-like polypeptide may also be used as a means for treating or preventing an HIV infection in a subject. Generally, the antibody binds to an HIV envelope like polypeptide having an amino acid sequence which is 80% or more identical to an amino acid sequence as depicted in FIG. 1 or FIG. 16. Suitable HIV envelope-like polypeptides include both mimotope and wild-type HIV envelope polypeptides.

In another aspect, the invention provides a detectable fusion peptide comprising or consisting essentially of a KLIC mimotope that is any one or more of SHGKLLA, TGKLQCI, QHSWSCSGKLLC, SLWTLHGSLISA, and SIWQTSGVLISY, or an immunogenic fragment thereof and a detectable reporter. In one embodiment, the fragment specifically binds a KLIC epitope-specific B cell.

In another aspect, the invention provides a method for isolating a B cell that specifically binds a KLIC motif of HIV, the method involving contacting a B cell with a detectable polypeptide containing a KLIC mimotope or immunogenic fragment thereof, thereby labeling the B cell, and isolating a B cell that specifically binds a KLIC motif of HIV. In various embodiments, the cells are isolated using flow cytometry, affinity purification, affinity chromatography, or column chromatography.

In yet another aspect, the invention is directed to a cell line that produces an antibody which specifically binds to one or more of SEQ ID NOs 1-84 and 94-109.

### BRIEF DESCRIPTION OF THE DRAWINGS

FIG. 1 illustrates HIV envelope-like polypeptides selected using serum from rhesus monkeys.

FIG. 2 shows the alignment of mimotopes selected with serum from monkey RK1-8 with the sequence of homologous Gp160_{SHIV-1157ip}. HIV envelope-like polypeptides are grouped according to their motifs (V3, gp120 C-terminus, gp41 immunodominant region (IDR), KLIC, and membrane proximal external region (MPER)) and aligned to gp160_{SHIV-1157ip} (grey rows). Linear homologies are shaded grey. Mimotopes are considered linear if they exhibited more than 50% linear amino acid identity.

FIG. 3 illustrates an amino acid sequence for gp160 and highlights the following domains: signal peptide, V1, V2, V3, V4, V5, C-terminus, fusion peptide, N-terminal heptad repeat region, immunodominant region, C-terminal heptad repeat domain, MPER, membrane-spanning domains 1 and 2, MSD3 and intracellular region.

FIG. 4 illustrates the location of mimotope A12.2 on Gp120. (Schreiber A, et al. J Comput Chem 26: 879-887, 2005). 3DEX analysis was used to find structural homology between A12.2 (yellow, orange and green) and the surface of gp120 (PDB ID: 2B4C) with the CD4 binding site shown in red and CCR5 coreceptor contact sites in blue. The inset table shows partial V3 sequences for SHIV-1157ip, the protein structure used for the 3DEX analysis (2B4C), and A12.2. Amino acid residues resembling the parental sequence but too few to form a linear epitope are shown in yellow and orange. Residues identified by 3DEX as showing 3D homology are shown in green.

FIG. 5 illustrates the conformational dependence of mimotope A12.2 by dot spot analysis with phage affinity-purified serum antibodies.

FIG. 6 illustrates the reactivity profile of mimotope fusion proteins by ELISA and Western Blot. (A) Mimotope fusion proteins were tested by ELISA with sera from monkeys RK1-8 and CF12. (B) Cross-reactivity of mimotope fusion protein AIV12.4 (26 kD; arrowhead) with a panel of rhesus monkey sera by Western blot.

FIG. 7 illustrates the immunizations and blood collection schedule for the mouse study of Example 5.

FIG. 8 Mouse immune sera were tested for binding to native (blue/orange bars) and reduced Env (black bars).

FIGS. 9A and 9B provide mimotope sequences (SEQ ID NOs 94-109) and alignments showing their similarity to the V3 loop or C terminal domain.

FIG. 10 is a schematic diagram that illustrates the selection of epitope-specific B cells using detectably labeled mimotopes.

FIG. 11 shows the isolation of the KLIC-specific B cells from REc-11 PBMC using a Fluorescence activated cell sorter.

FIG. 12 depicts gel images showing RT-PCR products of Igμ (510 bp; top panel), Igκ (350 bp; middle panel) and Igλ (350 bp; bottom panel) V genes amplified from single mimotope-specific rhesus monkey B cells. Each sample corresponds to a sample from a single sorted B cell.

FIG. 13 illustrates the immunizations and blood collection schedule for the rhesus monkey study of Example 11. Rhesus monkeys were primed once with DNA encoding the entire HIV-C envelope and subsequently boosted 5× with recombinant phage particles expressing mimotopes of HIV-C envelope. The rhesus monkeys were boosted with heterologous Gag-Pol particles twice and a third time in combination with another recombinant phage particle boost. The rhesus monkeys were challenged with a heterologous clade C virus in a 5 times low, one times high-dose regimen.

FIG. 14 is a table showing that cross-clade neutralizing antibodies were induced in rhesus monkeys. All sera showed high cross-clade serum neutralization titers in human PBMC assays in the presence of polymyxin B. Several animals showed also neutralization in the more commonly used TZM-b1 assay. Purified IgG confirmed the IgG-mediated neutralization activity in the serum samples.

FIG. 15 is a graph showing that partial protection during a multiple low-dose, heterologous SHIV challenge results in a lower peak viremia during acute infection. Plasma viral load during low-dose challenge is graphed up to 80 weeks after first low dose challenge. RHb-10+, RBb-10: immunized with phages encoding mimotope A12.2. RBc-10, RCc-10: immunized with V3 loop phages. RWc-10: immunized with C-terminal phages. During the low-dose challenge, several animals showed partial protection and/or lower peak viremia during acute infection.

FIG. 16 illustrates HIV envelope-like polypeptides selected using serum from rhesus monkeys.

### DETAILED DESCRIPTION OF THE INVENTION

The invention provides methods, compositions and kits for treating and or preventing diseases having immunological components associated with their etiology and/or progression.

The invention is based, at least in part, on the discovery of antibodies that recognize conformational epitopes on an antigen associated with a disease. Accordingly, the invention provides a protein, peptide, or fragment thereof that mimics the antigenicity of a reference polypeptide. Such antigenic mimics are referred to as mimotopes. The polypeptides and mimotopes of the invention are useful for inducing or enhancing an immune response to a disease. Such methods are useful for the treatment or prevention of virtually any disease having immunological components associated with its etiology and/or progression. In one embodiment, antibodies directed to the polypeptides and mimotopes of the invention may be administered to an individual to treat or prevent a disease and/or one or more symptoms associated with disease.

As detailed herein below, phage display was used to screen a random peptide libarary for binding to neutralizing antibodies obtained from monkeys that had been exposed to simian-human immunodeficiency virus (SHIV). To specifically select for mimic recognizing conformational antibody epitopes, RK1-8 serum was pre-incubated with native and in a separate reaction with denatured SHIV-1157ip gp160. Bound antibodies were eluted and used for a biopanning procedure with positive selections (using the antibodies eluted from the native gp160) and negative counter-selections (using the antibodies eluted from the denatured gp160. This method identified peptides resembling two different regions of gp120, the V3 loop and the C-terminal domain. Even though the V3 loop is very variable in sequence, antibodies targeting the conserved structure of this loop can be neutralizing. This is the first identification of conformational mimotopes of the V3 loop that induce neutralizing antibodies, which are of particular interest because they are expected to the HIV envelope structure. Significantly, the envelope mimics described herein (SEQ ID NOs: 94-109) are likely to induce antibodies that target conserved conformational epitopes on the HIV envelope glycoprotein and lead to potent neutralization by mimicking the complex three-dimensional structure rather than a linear amino acid sequence.

The invention further provides HIV envelope-like polypeptides (wild-type HIV polypeptides and mimotopes). These polypeptides and mimotopes may be administered to an individual so as to induce a protective immune response to HIV or to induce an immune response for the treatment of an existing HIV infection or AIDS. Alternatively, antibodies directed to the HIV envelope-like polypeptides may be administered to an individual to treat or prevent an HIV infection and/or one or more symptoms associated with the infection (e.g., AIDS). Mimotopes of the invention are also useful for the isolation of epitope specific B cells.

### DEFINITIONS

“HIV” is meant to include HIV-1 (human immunodeficiency virus type-1), HIV-2 (human immunodeficiency virus type-2) and SIV (simian immunodeficiency virus). HIV includes but is not limited to extracellular virus particles and the forms of HIV associated with HIV infected cells. “HIV” is Human Immunodeficiency Virus, a virus that causes immunodeficiency by attacking CD4⁺ cells in the body. HIV is organized into groups and subtypes (clades). Guidance on the application of the invention to different clades may also be found in HIV Sequence Compendium 2002 Kuiken C, Foley B, Freed E, Hahn B, Marx P, McCutchan F, Mellors J, Wolinsky S, and Korber B, editors. Published by the Theoretical Biology and Biophysics Group, Los Alamos National Laboratory, LA-UR number 03-3564, incorporated herein by reference. In particular, consensus sequence data for HIV-1 clades A, B, C and D and a number of isolates can be found on pages 490 to 550; consensus sequence data for HIV-2 clades A, B, C and D and a number of isolates can be found one pages 554 to 578.

As used herein, the terms “Env polypeptide” or “envelope polypeptide” refers to a molecule derived from an HIV envelope protein. An exemplary Env polypeptide sequence is provided at SEQ ID NO:93. The envelope protein of HIV is a glycoprotein of about 160 kd (gp160). During virus infection of the host cell, gp160 is cleaved by host cell proteases to form gp120 and the integral membrane protein, gp41. The gp41 portion is anchored in (and spans) the membrane bilayer of the virion, while the gp120 segment protrudes into the surrounding environment. Env polypeptides can exist as monomers, dimers or multimers.

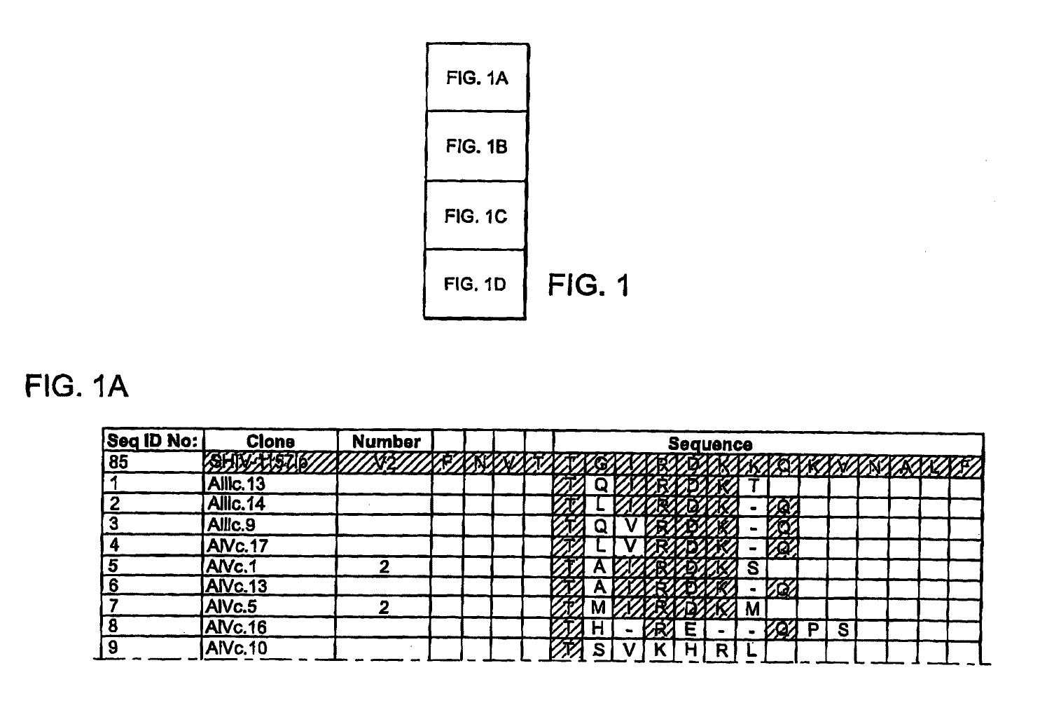

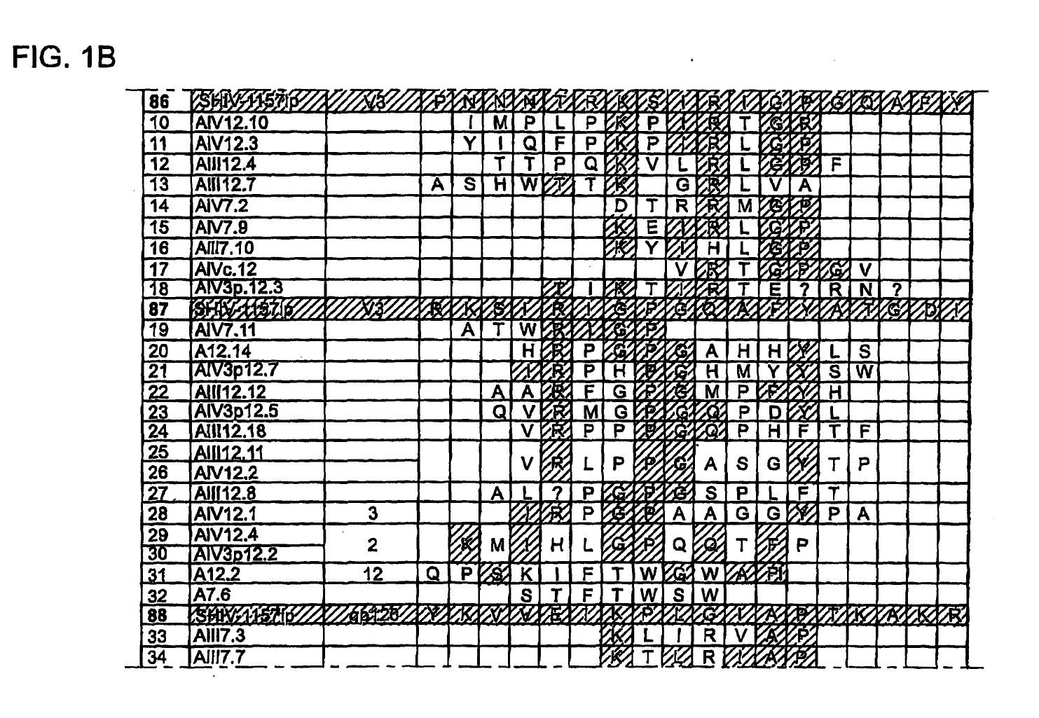

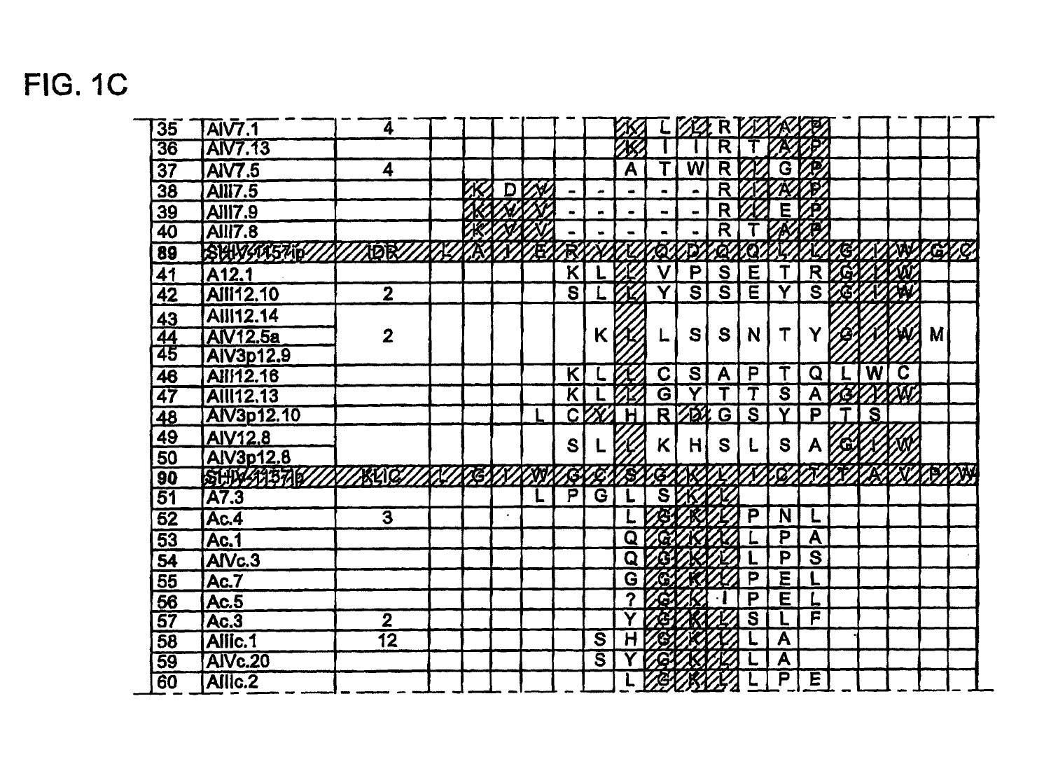

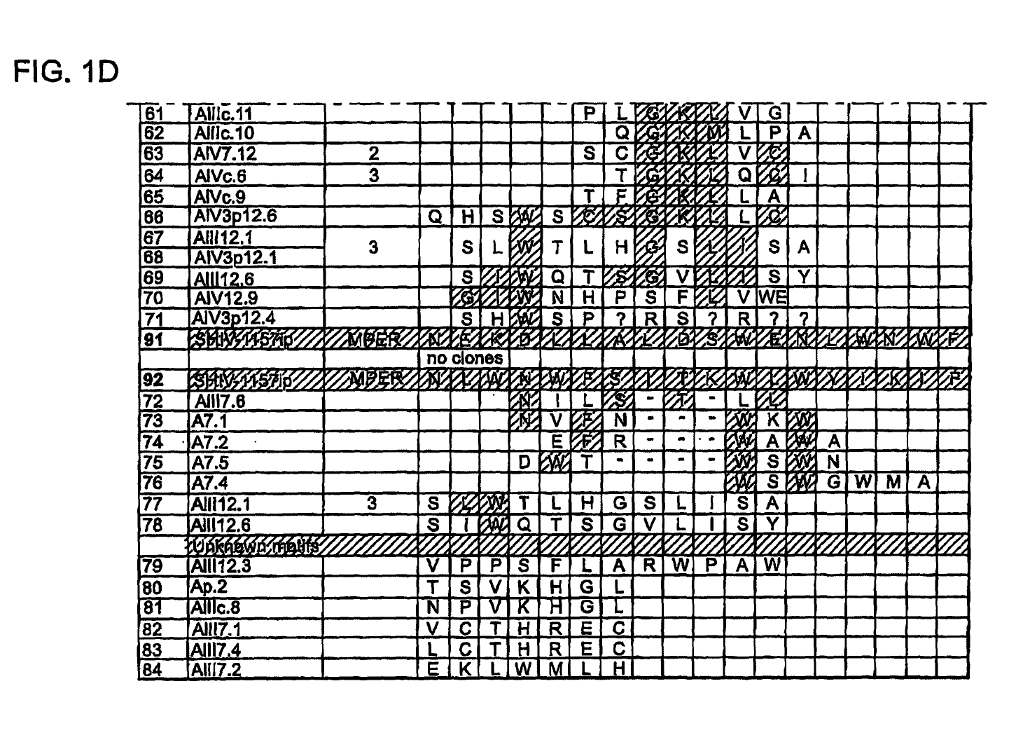

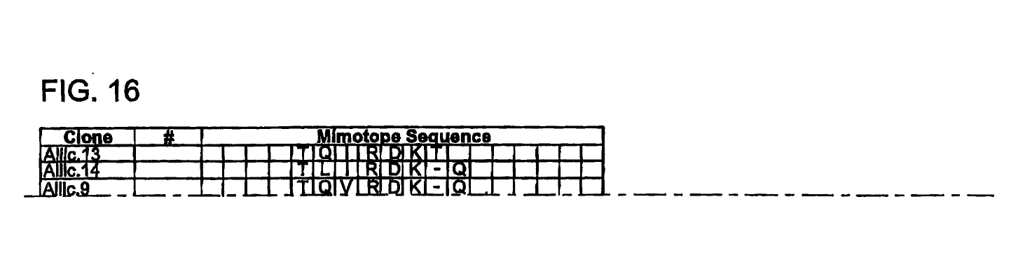

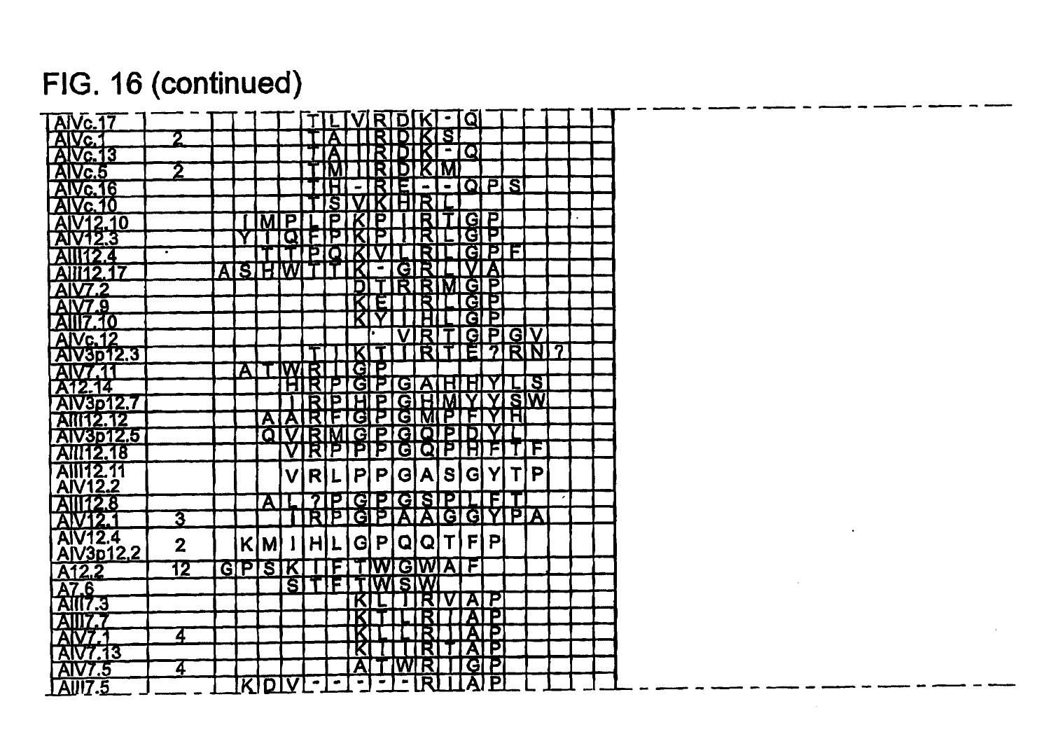

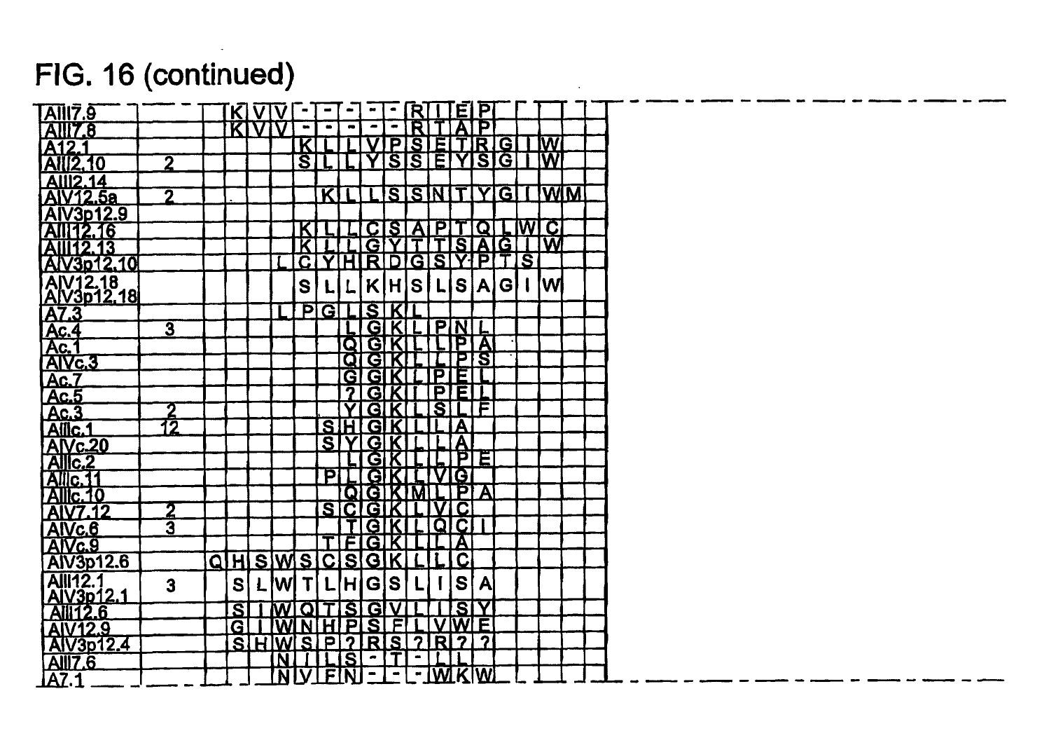

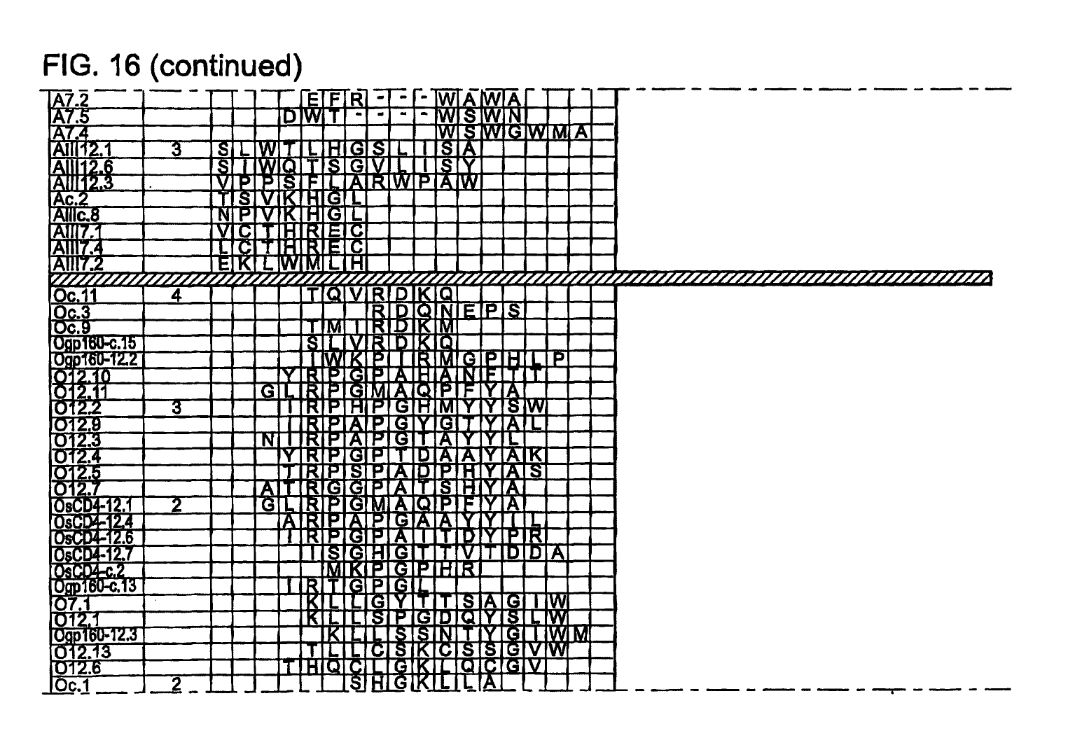

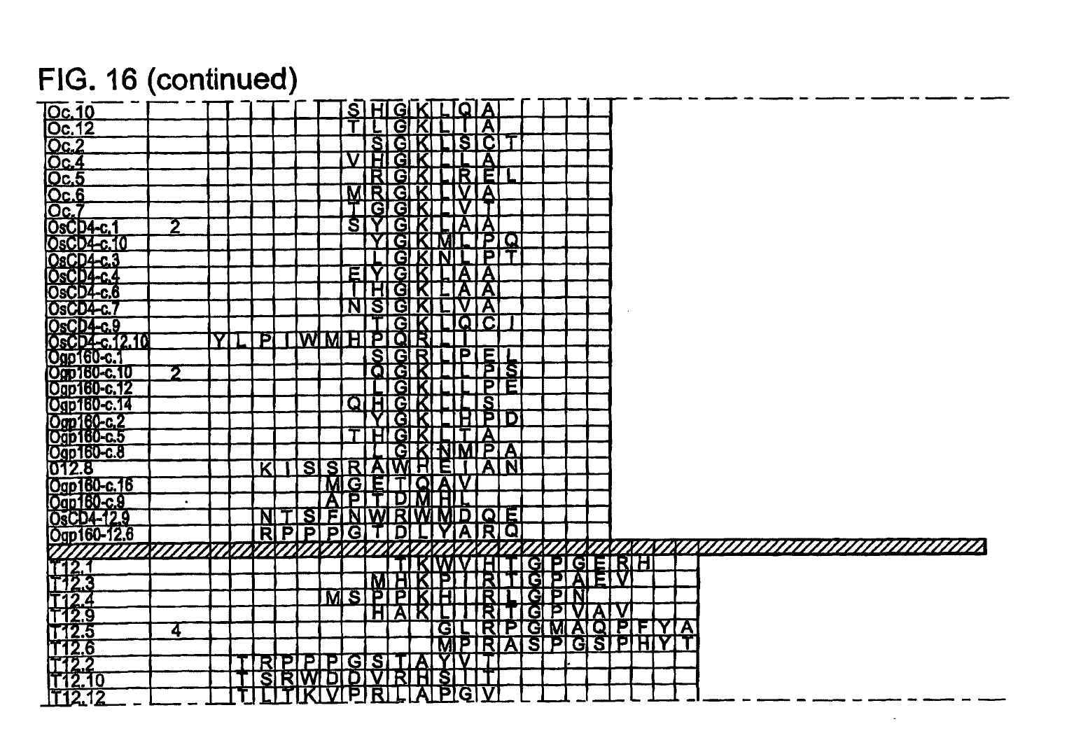

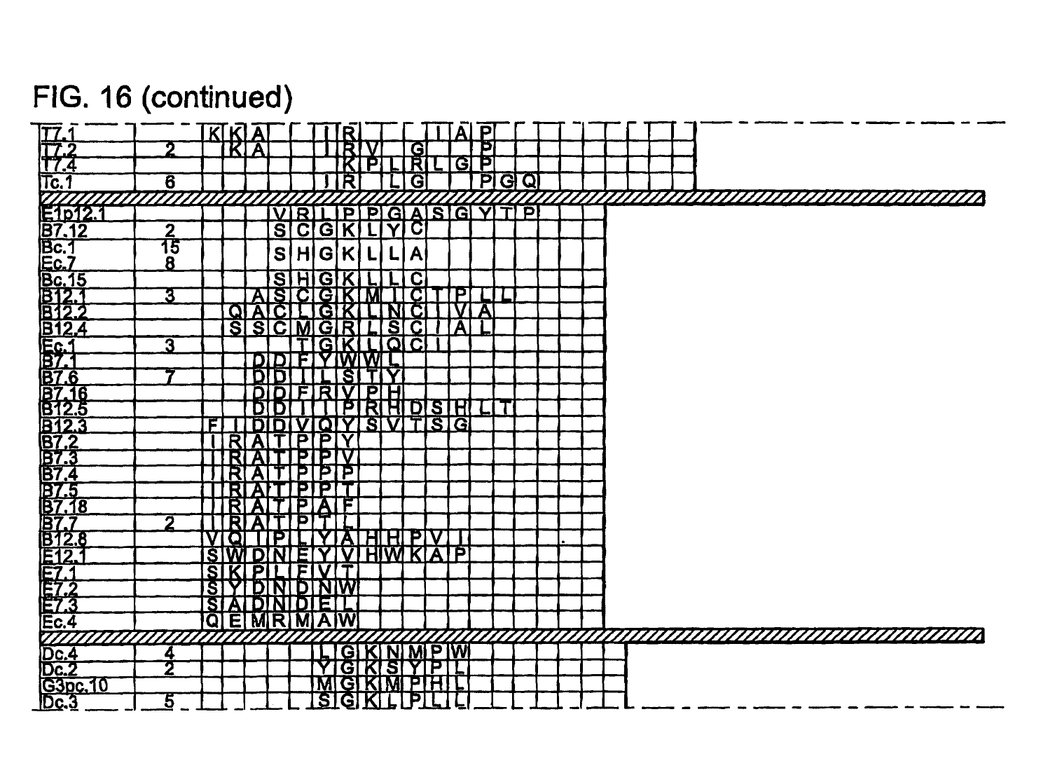

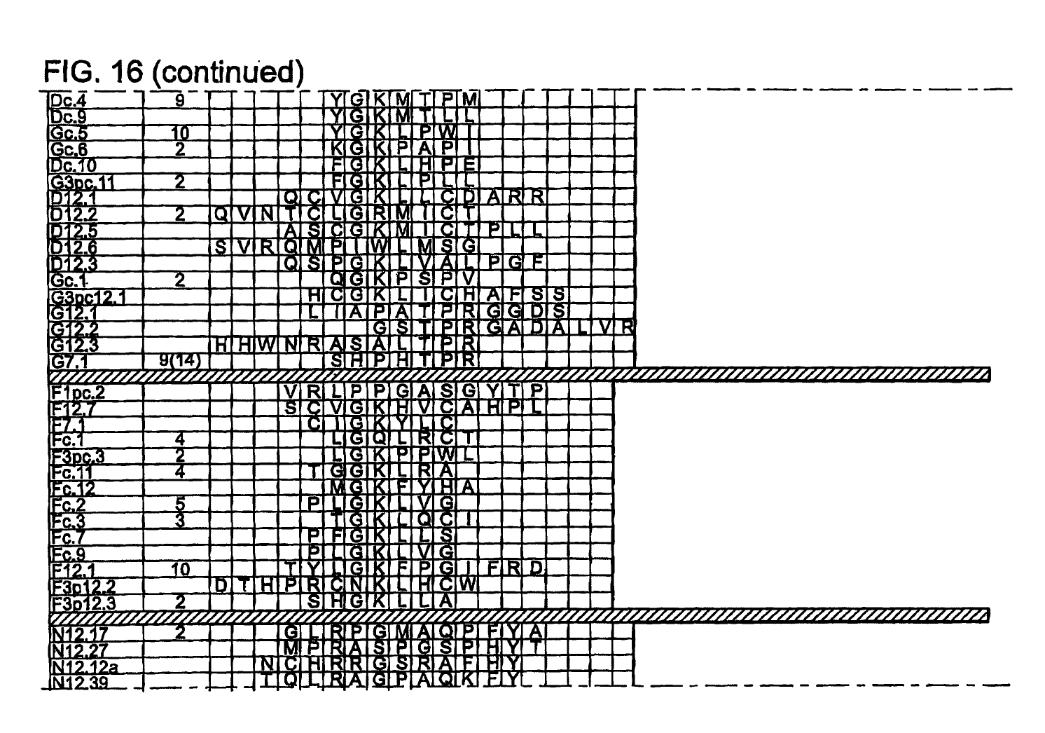

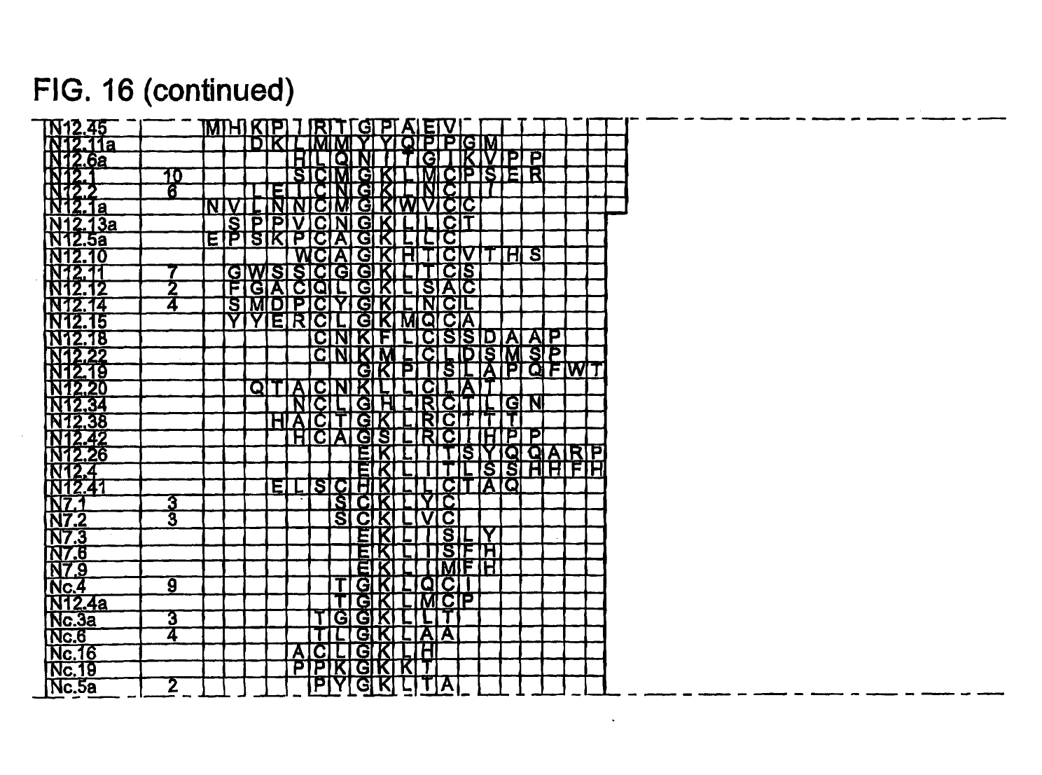

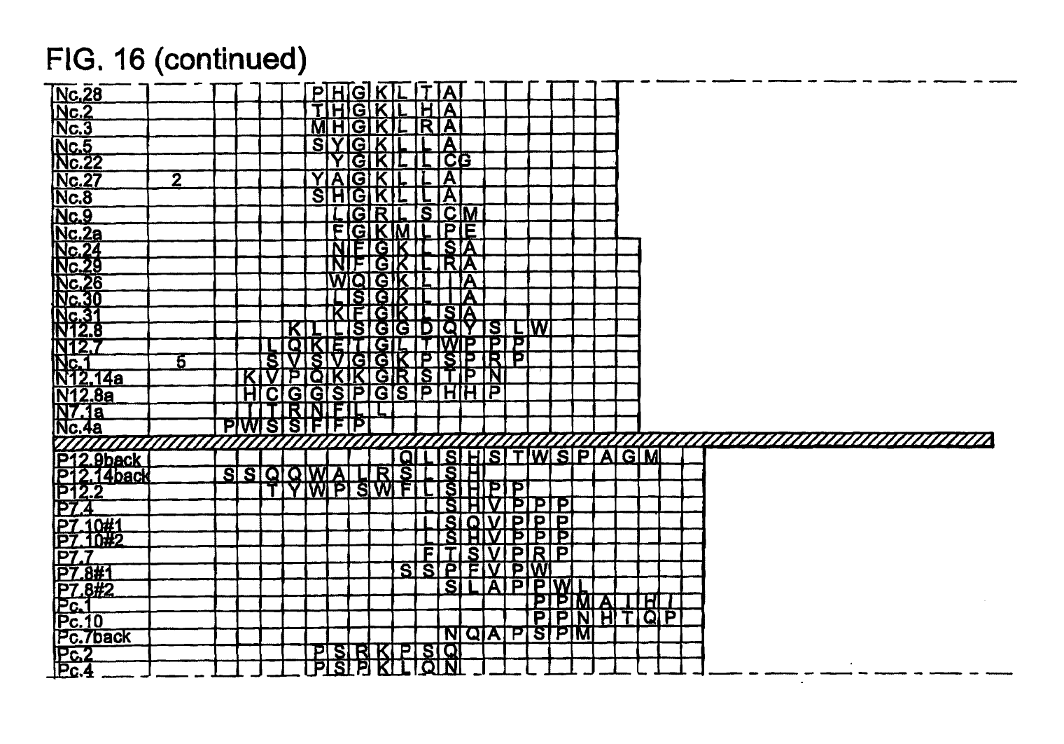

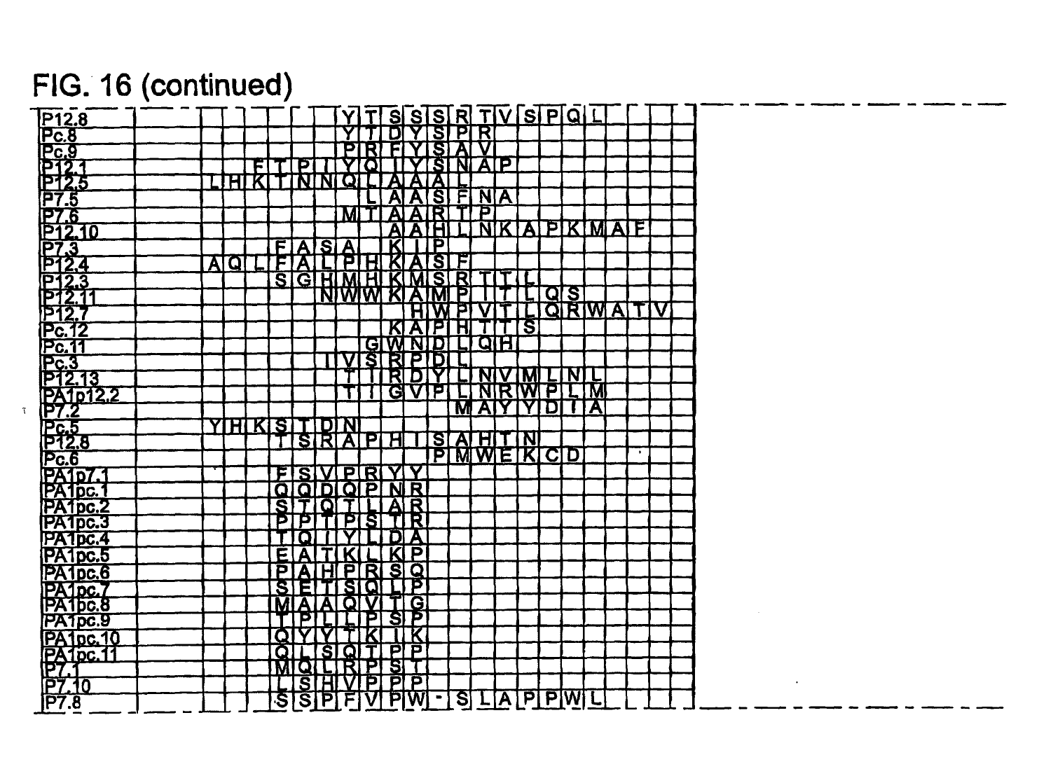

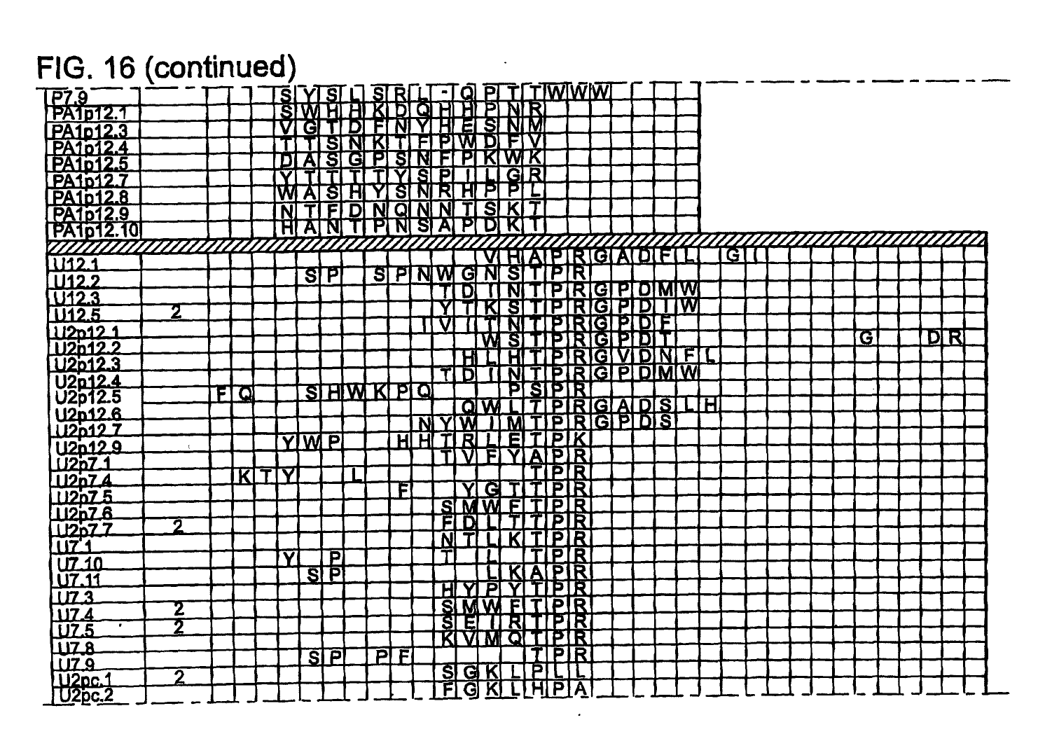

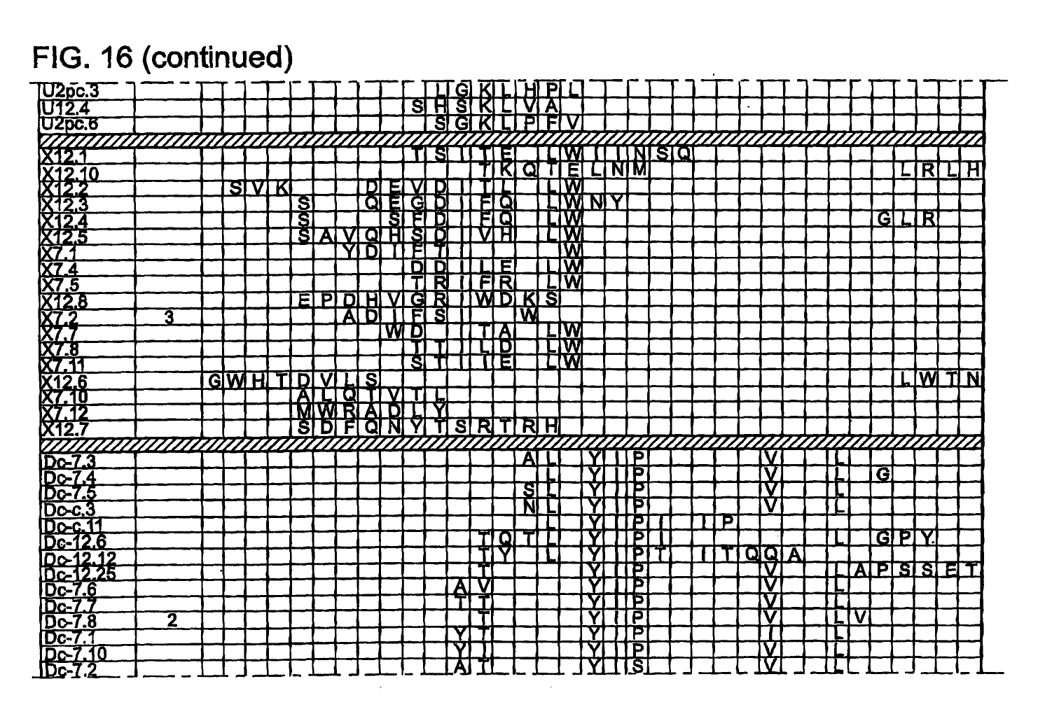

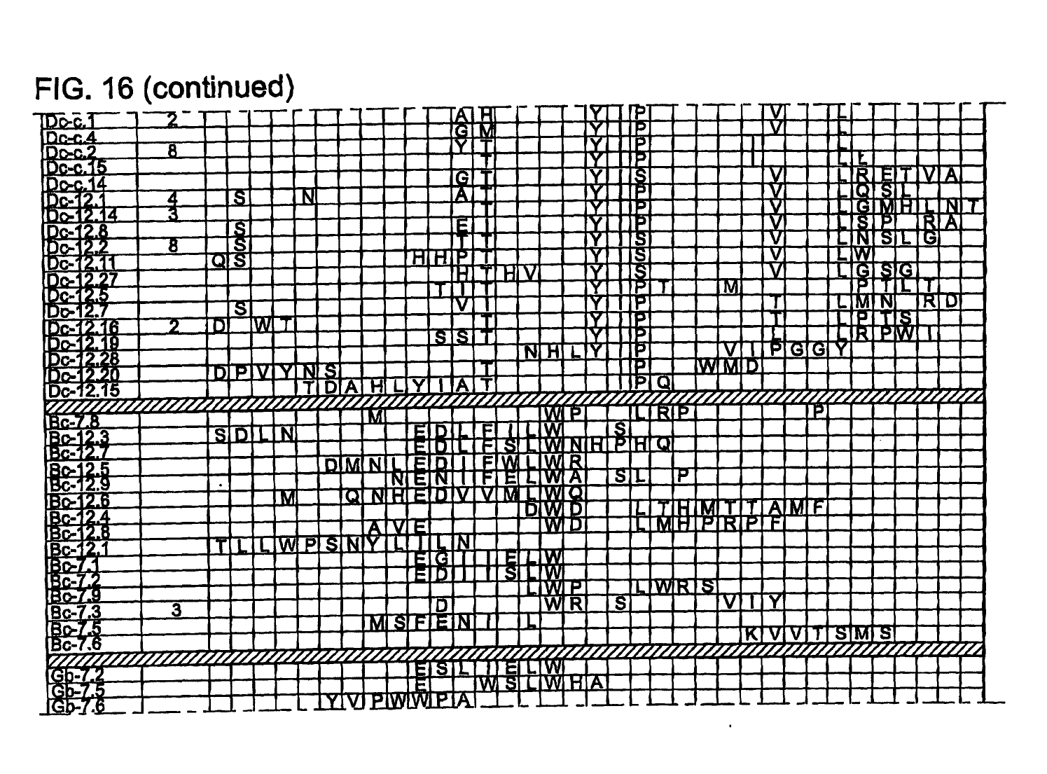

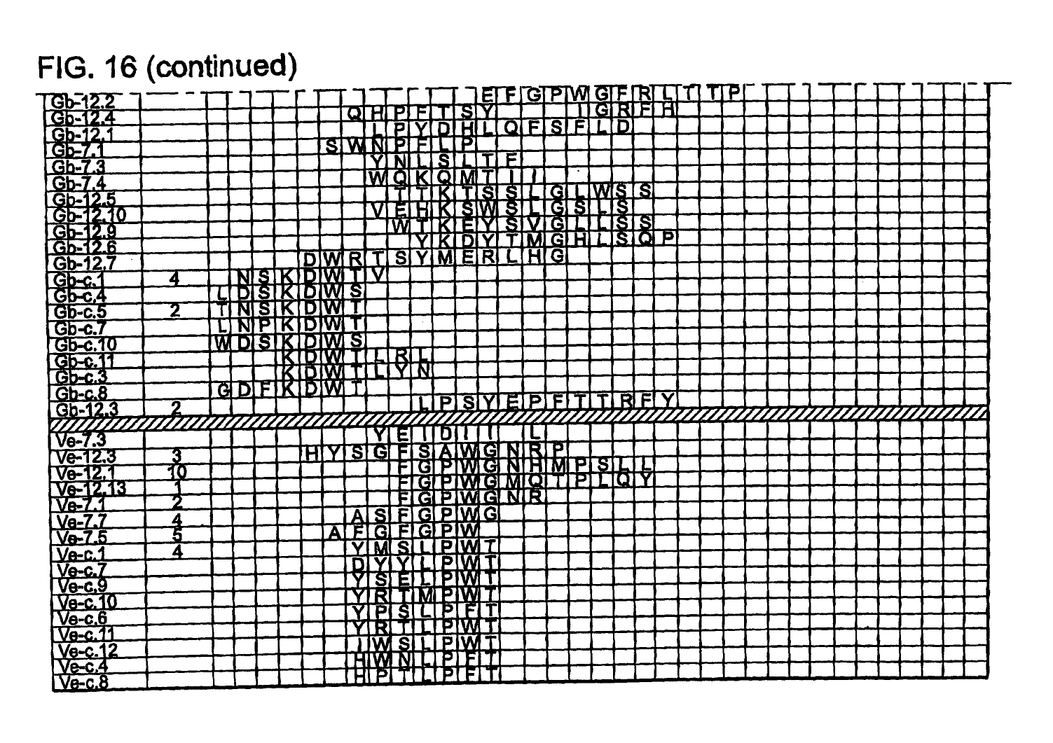

As used herein, an “HIV envelope-like polypeptide” refers to a polypeptide derived from an envelope polypeptide of HIV. HIV envelope-like polypeptides include both wild-type polypeptides (naturally occurring, e.g., isolated from HIV infected patients) and mimotopes. For example, HIV envelope-like polypeptides include those illustrated in FIG. 1 or FIG. 16. Additional HIV envelope like polypeptides are known in the art.

A “gp120 polypeptide” is a molecule derived from a gp120 region of a Env polypeptide. For example, a gp120 polypeptide includes a polypeptide corresponding to amino acid residues 1-511 of SEQ ID NO:93. The amino acid sequence of gp120 is approximately 511 amino acids. gp120 contains five relatively conserved domains interspersed with five variable domains (V1-V5). The variable domains contain extensive amino acid substitutions, insertions and deletions. A “gp120 polypeptide” includes both single subunits and multimers.

An “HIV V3 region” is the third of five variable loop regions of gp120 and is involved in viral infectivity by binding to a cytokine receptor on the cellular target. For example, binding of the CCR5 or CXCR4 cytokine receptors to a target. The HIV V3 regions are known in the art and described herein. For example, an HIV V3 region corresponds to amino acid residues 296-331 of SEQ ID NO:93.

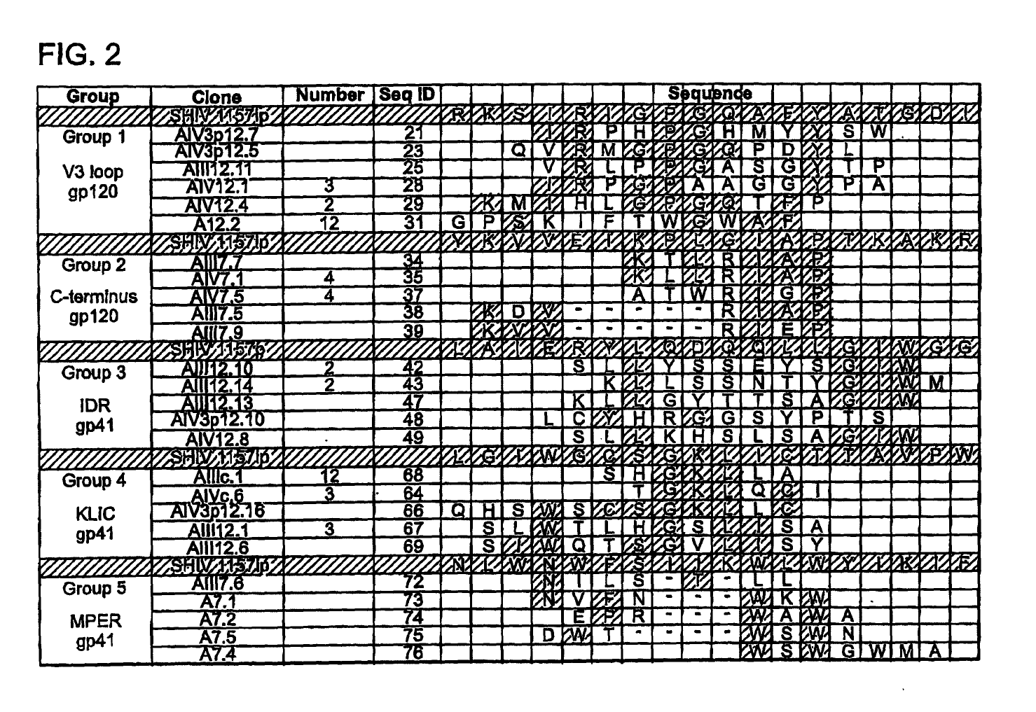

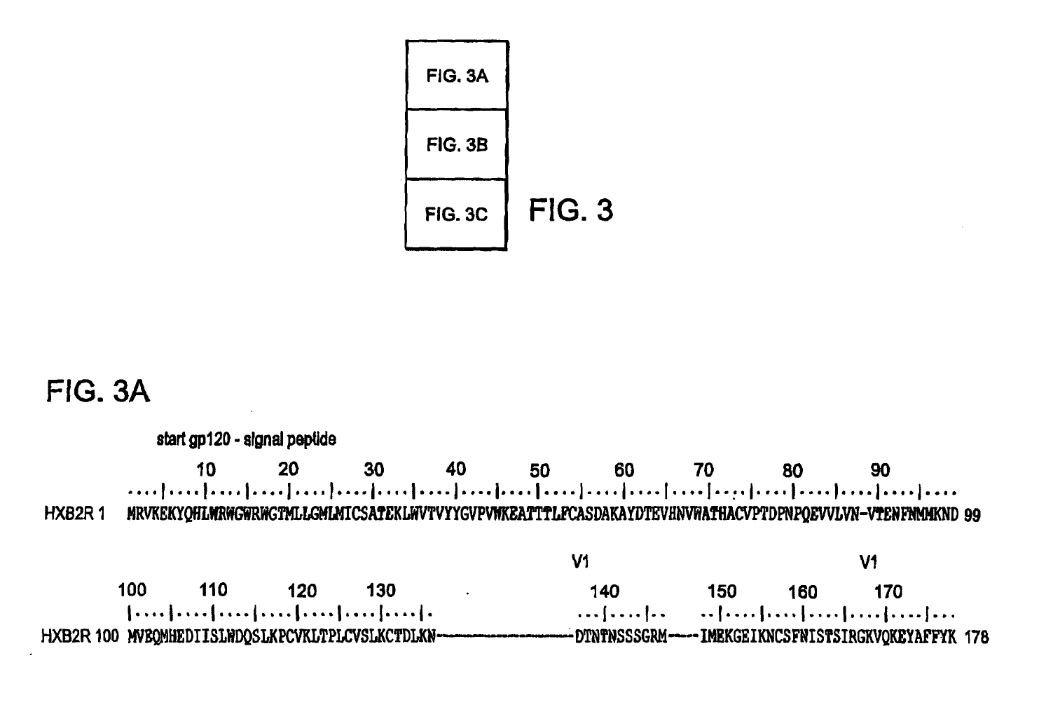

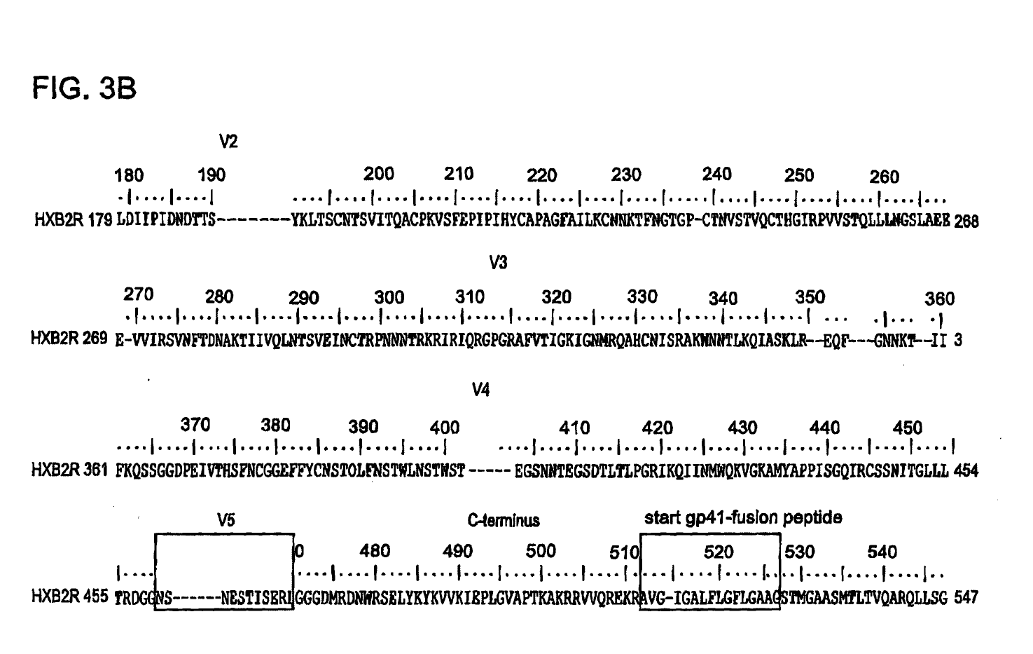

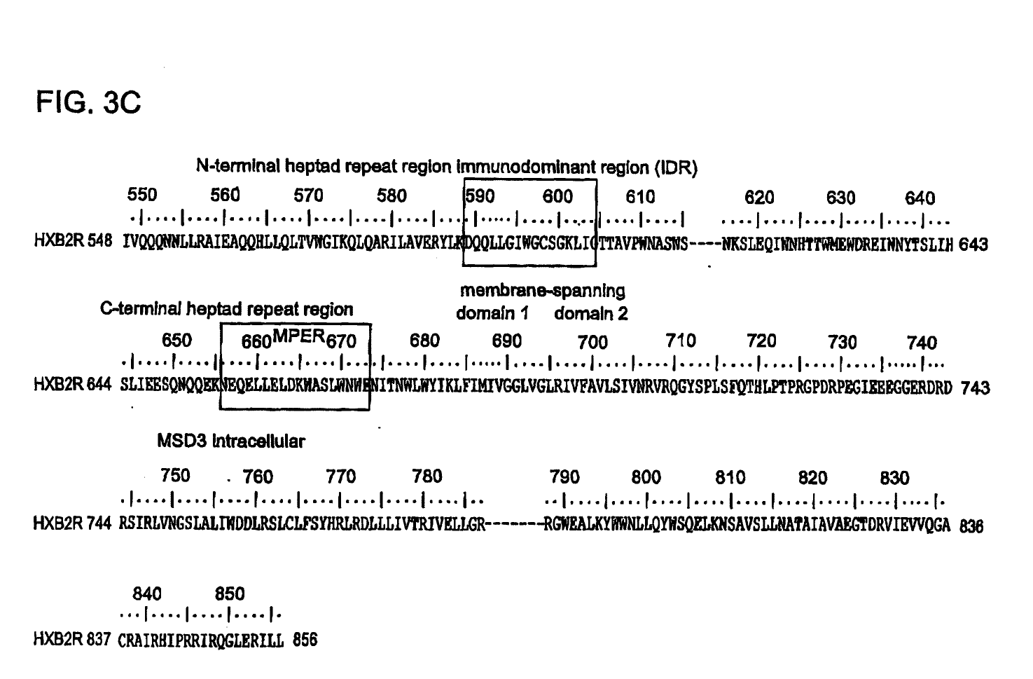

By “V3 loop mimotope” is meant a polypeptide, peptide, or portion thereof that binds an antibody generated against a V3 loop motif. The sequences of exemplary V3 loop mimotopes are provided at FIG. 2.

An “HIV C-terminal envelope region” is the C-terminal portion of the gp120 protein of HIV. C-terminal envelope polypeptides are known in the art and described herein. The C-terminal envelope region of HIV corresponds to amino acid residues ˜485-509 of SEQ ID NO:93.

By “HIV V3 loop mimotopes” is meant a polypeptide, peptide, or portion thereof that binds an antibody generated against an HIV V3 loop. The sequences of exemplary HIV V3 loop mimotopes are provided at FIG. 2.

By “IDR mimotope” is meant a polypeptide, peptide, or portion thereof that binds an antibody generated against an HIV IDR motif. The sequences of exemplary IDR mimotopes are provided at FIG. 2.

By “HIV KLIC motif” is meant a portion of the HIV envelope comprising or consisting essentially of the following amino acid sequence: LGIWGCSGKLICTTAVPW.

By “KLIC mimotope” is meant a polypeptide, peptide, or portion thereof that binds an antibody generated against an HIV KLIC motif. The sequences of exemplary KLIC mimotopes are provided at FIG. 2.

By “MPER mimotope” is meant a polypeptide, peptide or portion thereof that binds an antibody generated against an MPER motif. The sequences of exemplary MPER mimotopes are provided at FIG. 2.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified.

The term “mimotope” refers to a string of amino acids that mimics the structural features of a reference polypeptide or fragment thereof (e.g., a subdomain or specific domain of an antigen). A mimotope can bind an antibody that is specific for a given epitope of an antigen of interest; the mimotope binds to an antigen specific antibody because of structural mimicry rather than primary amino acid sequence identity with the antigen's epitope. In one embodiment, the term “mimotope” refers to a polypeptide that differs from an envelope polypeptide by one or more amino acids, but which mimics the three dimensional structure of a wild-type envelope epitope. A mimotope—generally in the context of a larger protein backbone called a carrier—is able to stimulate a host's immune system to induce an antigen-specific response that generates antibodies that specifically bind to the mimotope and the corresponding wild-type envelope epitope. Mimotopes of the invention include the polypeptides of SEQ ID Nos: 1-84 and 94-109.

As used herein, “antigen” refers to a molecule containing one or more epitopes that will stimulate a host's immune system to make a humoral and/or cellular antigen-specific response. Epitopes are linear, conformational or both. As used herein, the term “epitope” is used interchangeably with the term “mimotope.” Normally, a B-cell epitope will include at least about 5 amino acids, but it may be as small as about 3-4 amino acids. Many B-cell epitopes are discontinuous. A T-cell epitope, such as a CTL epitope, will include at least about 7-9 amino acids, and a helper T-cell epitope will include at least about 12-20 amino acids. Normally, an epitope will include between about 7 and 15 amino acids, such as, 9, 10, 12 or 15 amino acids. Antigens of the present invention include the polypeptides shown in FIG. 1 or FIG. 16.

By “active immunization” is meant the administration of an antigen, epitope or mimotope to induce antibodies in an individual to the antigen, epitope, or mimotope, e.g., to prevent or treat infections or disease.

By “passive immunization” is meant the administration of monoclonal or polyclonal antibodies to an individual to prevent or treat infections or disease.

By “marker” is meant any protein or polynucleotide having an alteration in expression level or activity that is associated with a disease or disorder. A marker may be an antigen.

By “pathogen” is meant any bacteria, viruses, fungi, protozoans, or parasites capable of interfering with the normal function of a cell.

Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus Bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Fungi, including, but not limited to Candida, cause invasive diseases in hosts with altered immunity, such as patients with HIV infection, organ or bone marrow transplants, or neutropenia following cancer immunotherapy. There are approximately 200 species of the genus Candida, but nine cause the great majority of human infections. They are C. albicans, C. krusei, C. glabrata (formerly called Torulopsis glabrata), C. parapsilosis, C. tropicalis, C. pseudotropicalis, C. guilliermondii, C. dubliniensis, and C. lusitaniae. They cause infections of the mucous membranes, for example, thrush, esophagitis, and vagititis; skin, for example, intertrigo, balanitis, and generalized candidiasis; blood stream infections, for example, candidemia; and deep organ infections, for example, hepatosplenic candidiasis, urinary tract candidiasis, arthritis, endocarditis, and endophthamitis.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Parasites are organisms which depend upon other organisms in order to survive and thus must enter, or infect, another organism to continue their life cycle. The infected organism, i.e., the host, provides both nutrition and habitat to the parasite. The term “parasite” as used herein refers to protozoa, helminths, and ectoparasitic arthropods (e.g., ticks, mites, etc.). Protozoa are single celled organisms which can replicate both intracellularly and extracellularly, particularly in the blood, intestinal tract or the extracellular matrix of tissues. Helminths are multicellular organisms which almost always are extracellular (the exception being Trichinella). Helminths normally require exit from a primary host and transmission into a secondary host in order to replicate. In contrast to these aforementioned classes, ectoparasitic arthropods form a parasitic relationship with the external surface of the host body.

Parasites can be classified based on whether they are intracellular or extracellular. An “intracellular parasite” as used herein is a parasite whose entire life cycle is intracellular. Examples of human intracellular parasites include Leishmania, Plasmodium, Trypanosoma cruzi, Toxoplasma gondii, Babesia, and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at lest one obligate intracellular stage in their life cycles. This latter category of parasites includes Trypanosoma rhodesiense and Trypanosoma gambiense, Isospora, Cryptosporidium, Eimeria, Neospora, Sarcocystis, and Schistosoma. In one aspect, the invention relates to the prevention and treatment of infection resulting from intracellular parasites and obligate intracellular parasites which have at least in one stage of their life cycle that is intracellular. In some embodiments, the invention is directed to the prevention of infection from obligate intracellular parasites which are predominantly intracellular. An exemplary and non-limiting list of parasites for some aspects of the invention is provided herein.

Blood-borne and/or tissues parasites include Plasmodium, Babesia microti, Babesia divergens, Leishmania tropica, Leishmania, Leishmania braziliensis, Leishmania donovani, Trypanosoma gambiense and Trypanosoma rhodesiense (African sleeping sickness), Trypanosoma cruzi (Chagas' disease), and Toxoplasma gondii.

Parasites include parasitic helminths such as nematodes, cestodes, and trematodes. Preferred nematodes to target include filariid, ascarid, capillarid, strongylid, strongyloides, trichostrongyle, and trichurid nematodes. Particularly preferred nematodes are those of the genera Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parajilaria, Paragonimus, Parascaris, Physaloptera, Protostrongylus, Setaria, Spirocerca, Spirometra, Stephanofilaria, Strongyloides, Strongylus, Thelazia, Toxascaris, Toxocara, Trichinella, Trichostrongylus, Trichuris, Uncinaria, and Wuchereria. Preferred filariid nematodes include Dirofilaria, Onchocerca, Acanthocheilonema, Brugia, Dipetalonema, Loa, Parafilaria, Setaria, Stephanofilaria and Wuchereria filariid nematodes, with D. immitis being even more preferred.

As used herein, an “antibody” includes any reactive fragment or fragments of antibodies such as Fab molecules, Fab proteins, single chain polypeptides, or the multi-functional antibodies having binding affinity for an antigen. The term includes chimeric antibodies, altered antibodies, univalent antibodies, bi-specific antibodies, monoclonal antibodies, polyclonal antibodies, human antibodies and humanized antibodies. Methods for preparing antibodies are well known in the art and described herein. A “neutralizing antibody” is an antibody that prevents HIV infection of target cells.

By “binding to” a molecule is meant having a physicochemical affinity for that molecule.

By “specifically binds” as it refers to an antibody means that the antibody recognizes and binds a polypeptide of the invention, but does not substantially recognize and bind other molecules in a sample. In general, an antibody that specifically binds an immunogen binds with high avidity and/or high affinity. Antibody binding to its epitope on a specific antigen is stronger than binding of the same antibody to any other epitope (with the general exception of a wild-type HIV env and its corresponding mimotope), particularly those which may be present in molecules in association with, or in the same sample, as the specific antigen of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g., by use of appropriate controls.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “humoral immune response” or “antibody immune response” refers to an immune response mediated by antibody molecules, while a “cellular immune response” is one mediated by T lymphocytes and/or other white blood cells. The antigen of interest may elicit an antibody-mediated immune response. Hence, an immunological response may include one or more of the following effects: the production of antibodies by B cells; and/or the activation of suppressor, cytotoxic, or helper T cells and/or T cells directed specifically to an antigen or antigens present in the composition or vaccine of interest. These responses may serve to neutralize infectivity, and/or mediate antibody-mediated complement lysis, or antibody dependent cell cytotoxicity (ADCC) to provide protection to an immunized host. Such responses can be determined using standard immunoassays and neutralization assays which are well known in the art.

As used herein, “isolated” when referring to a polynucleotide or a polypeptide means that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose.

The term “amino acid” refers to a molecule containing both an amino group and a carboxyl group. Suitable amino acids include, without limitation, both the D- and L-isomers of the 20 common naturally occurring amino acids found in peptides (e.g., A, R, N, C, D, Q, E, G, H, I, L, K, M, F, P, S, T, W, Y, V (as known by the one letter abbreviations)) as well as the naturally occurring and unnaturally occurring amino acids prepared by organic synthesis or other metabolic routes.

A “conservative amino acid substitution” is one in which the amino acid residue is replaced with an amino acid residue having a similar side chain. For example, families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Other conserved amino acid substitutions can also occur across amino acid side chain families, such as when substituting an asparagine for aspartic acid in order to modify the charge of a peptide.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 3, 4, 5, 6, 7, 8, 9, 10, 20, 30, 40, 50, or 60 nucleotides or amino acids.

As used herein a “polypeptide” is a molecular chain of amino acids and does not refer to a specific length of the product. Thus, peptides, oligopeptides dimers, multimers, and the like are included within the definition of polypeptide. This term is also intended to include polypeptides that have been subjected to post-expression modifications such as, for example, glycosylations, acetylations, phosphorylations and the like. Furthermore, for purposes of the present invention, a “polypeptide” refers to a protein that includes modifications, such as deletions, additions and substitutions (generally conservative in nature), to the native sequence, as long as the protein maintains the desired activity.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “reporter gene” is meant a gene encoding a polypeptide whose expression may be assayed; such polypeptides include, without limitation, glucuronidase (GUS), luciferase, chloramphenicol transacetylase (CAT), and beta-galactosidase.

“Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of I and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Similarity” or “percent similarity” in the context of two or more polypeptide sequences, refer to two or more sequences or subsequences that are the same or have a specified percentage of amino acid residues, or conservative substitutions thereof, that are the same when compared and aligned for maximum correspondence, as measured using one of the following sequence comparison algorithms, or by visual inspection.

By “subject” is meant a mammal, including, but not limited to, a human or non-human mammal, such as a bovine, equine, canine, ovine, rodent, or feline. By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

In one aspect, the invention is directed to an isolated HIV envelope-like polypeptide. Generally, the HIV envelope-like polypeptide has an amino acid sequence which is 80% or more identical to a reference sequence. In one embodiment, the reference sequence is the amino acid sequence depicted in FIG. 1 or FIG. 16. Suitable HIV envelope-like polypeptides include both mimotope and wild-type HIV envelope polypeptides (gp160). In some embodiments, the HIV envelope-like polypeptides have an amino acid sequence which is 85%, 90%, 95%, 96%, 97%, 98%, 99% or more identical to a reference sequence, for example, an amino acid sequence depicted in FIG. 1 (SEQ ID NOs: 1-84), FIG. 16, or provided at SEQ ID NOs 94-109. In a preferred embodiment, the HIV envelope-like polypeptide has an amino acid sequence which is identical to an amino acid sequence depicted in FIG. 1 or FIG. 16. HIV envelope-like peptides which are mimotopes are illustrated in FIG. 1 as SEQ ID NOs: 1-84; of these SEQ ID NOs: 21, 23, 25, 28, 29 and 31 correspond to the HIV V3 envelope region and SEQ ID NOs: 34, 35, 37, 38 and 39 correspond to the HIV envelope C-terminal region. The HIV envelope-like polypeptides may also be wild-type HIV envelope proteins, for example, peptides that are 80% or more identical to SEQ ID NOs: 85-93. In some instances, the HIV envelope-like polypeptide may be a fusion polypeptide, e.g., mimotope linked to a carrier polypeptide. In other embodiments, a polypeptide of the invention is a polypeptide comprising a motif shown in FIG. 2, or is a polypeptide, peptide or fragment thereof that comprises or consists essentially of a mimotope listed in FIG. 2. In still other embodiments, the polypeptide, peptide or fragment thereof comprises or consists essentially of a KLIC mimotope that is SHGKLLA, TGKLQCI, QHSWSCSGKLLC, SLWTLHGSLISA, or SIWQTSGVLISY. In other embodiments, the invention provides fusion polypeptides comprising a mimotope of the invention linked to a detectable moiety. In one embodiment, the fusion polypeptide comprises a mimotope of the invention linked to a detectable reporter. In still other embodiments, the invention provides a KLIC mimotope (e.g., SHGKLLA, TGKLQCI, QHSWSCSGKLLC, SLWTLHGSLISA, or SIWQTSGVLISY) linked to a detectable reporter (e.g., GFP, YFP, BFP, CFP).

The invention is also directed to nucleic acids encoding the HIV envelope-like polypeptides. Generally, the nucleic acids encode HIV envelope-like polypeptides having an amino acid sequence which is 80% or more identical to an amino acid sequence depicted in FIG. 1 or FIG. 16. Suitable HIV envelope-like polypeptides include both mimotope and wild-type HIV envelope polypeptides (gp160). In some embodiments, the nucleic acids encode HIV envelope-like polypeptides having an amino acid sequence which is 85%, 90%, 95%, 96%, 97%, 98%, 99% or more identical to an amino acid sequence depicted in FIG. 1 or FIG. 16. In a preferred embodiment, the nucleic acid encodes an envelope-like polypeptide having an amino acid sequence which is identical to an amino acid sequence depicted in FIG. 1 or FIG. 16. HIV envelope-like peptides which can be characterized as mimotopes are illustrated in FIG. 1 as SEQ ID NOs: 1-84; of these SEQ ID NOs: 21, 23, 25, 28, 29 and 31 correspond to the HIV V3 envelope region and SEQ ID NOs: 34, 35, 37, 38 and 39 correspond to the HIV envelope C-terminal region. The nucleic acids may encode HIV envelope-like polypeptides which are wild-type HIV envelope proteins, for example, peptides that are 80% or more identical to SEQ ID NOs: 85-93. In some instances, the nucleic acid encodes a HIV envelope-like polypeptide which is a fusion polypeptide, e.g., mimotope linked to a carrier polypeptide. The nucleic acids may be isolated, in a vector or expressed in a host cell.

In yet another aspect the invention is directed to an antibody that specifically binds to an HIV envelope-like polypeptide. Generally, the antibody will bind to a HIV envelope-like polypeptide having an amino acid sequence which is 80% or more identical to an amino acid sequence depicted in FIG. 1 or FIG. 16. Suitable antibodies bind to HIV envelope-like polypeptides, including mimotope and wild-type HIV envelope polypeptides (gp160). In some embodiments, the antibodies bind to HIV envelope-like polypeptides which have an amino acid sequence which is 85%, 90%, 95%, 96%, 97%, 98%, 99% or more identical to an amino acid sequence depicted in FIG. 1, FIG. 16, or provided at SEQ ID NOs 94-109. In a preferred embodiment, the antibody binds to an envelope-like polypeptide having an amino acid sequence which is identical to an amino acid sequence depicted in FIG. 1, FIG. 16, or provided at SEQ ID NOs 94-109. In one embodiment, the antibody binds to a mimotope of an HIV envelope peptide such as those illustrated in SEQ ID NOs: 1-84 or 94-109. More specifically the antibody may bind to a mimotope of the HIV V3 envelope region, wherein the mimotope has the amino acid sequence of SEQ ID NOs: 21, 23, 25, 28, 29, 31, and 94-103. The antibodies of the invention may bind to HIV envelope-like peptides which correspond to the HIV envelope C-terminal region, e.g., SEQ ID NOs: 34, 35, 37, 38, 39, and 104-109. The antibodies may also be specific for and bind to wild-type HIV envelope proteins, for example, peptides that are 80% or more identical to SEQ ID NOs: 85-93.

The antibodies may be derived from a vertebrate such as a mouse, human or monkey and can be either polyclonal or monoclonal. Chimeric and humanized antibodies as well as binding fragments (e.g. Fab fragment) are also contemplated.

Further, any of the above HIV envelope-like polypeptides, nucleic acids and antibodies may be included in a kit or composition, e.g., pharmaceutical composition.

The invention is also directed to methods for inducing an immune response in a subject by administering a pharmaceutical composition having an immunologically effective dose of one or more HIV envelope-like polypeptides. Generally, the HIV envelope-like polypeptide is 80% or more identical to an amino acid sequence of FIG. 1 or FIG. 16. Suitable HIV envelope-like polypeptides include both mimotope and wild-type HIV envelope polypeptides (gp160). In some embodiments, the HIV envelope-like polypeptide has an amino acid sequence which is 85%, 90%, 95%, 96%, 97%, 98%, 99% or more identical to an amino acid sequence depicted in FIG. 1, FIG. 16, or provided at SEQ ID NOs 94-109. In a preferred embodiment, the subject is administered one or more envelope-like polypeptides having an amino acid sequence which is identical to an amino acid sequence depicted in FIG. 1, FIG. 16, or provided at SEQ ID NOs 94-109. Peptides suitable for inducing a protective immune response include HIV envelope-like peptides having the amino acid sequence of SEQ ID NOs: 1-84, of these SEQ ID NOs: 21, 23, 25, 28, 29, 31, and 94-103 correspond to HIV V3 envelope region SEQ ID NOs: 34, 35, 37, 38, 39, and 104-109 correspond to the HIV envelope C-terminal region. Alternatively, the HIV envelope-like polypeptides included in the pharmaceutical composition may be wild-type HIV envelope proteins, for example, peptides that are 80% or more identical to SEQ ID NOs: 85-93. Preferably, the HIV envelope-like polypeptide is fusion polypeptide, e.g., mimotope-carrier fusion polypeptide. In some embodiments, the method of inducing an immune response includes the sequential or co-administration of a nucleic acid encoding a HIV envelope polypeptide (e.g., gp160).

Administering a therapeutically effective dose of an antibody that specifically binds to an HIV envelope-like polypeptide may also be used as a means for treating or preventing an HIV infection in a subject. Generally, the HIV envelope like polypeptide has an amino acid sequence which is 80% or more identical to an amino acid sequence depicted in FIG. 1, FIG. 16, or provided at SEQ ID NOs 94-109. Suitable antibodies bind to HIV envelope-like polypeptides include both mimotope and wild-type HIV envelope polypeptides (gp160). In some embodiments, the antibodies bind to HIV envelope-like polypeptides having an amino acid sequence which is 85%, 90%, 95%, 96%, 97%, 98%, 99% or more identical to an amino acid sequence depicted in FIG. 1, FIG. 16, or provided at SEQ ID NOs 94-109. In a preferred embodiment, the antibody binds to an envelope-like polypeptide having an amino acid sequence which is identical to an amino acid sequence depicted in FIG. 1, FIG. 16, or provided at SEQ ID NOs 94-109. In one embodiment, the antibody binds to a mimotope of an HIV envelope peptide such as those illustrated in SEQ ID NOs: 1-84 and 94-109. More specifically the antibody binds to a mimotope of the HIV V3 envelope region, wherein the mimotope has an amino acid sequence which is 80% or more identical to SEQ ID NOs: 21, 23, 25, 28, 29, 31, and 94-103. In another embodiment, the antibody used in the method of treatment is a mimotope derived from the HIV C-terminal envelope region and has an amino acid sequence which is 80% or more identical to the amino acid sequence of SEQ ID NOs: 34, 35, 37, 38, 39, and 104-109. The antibodies used in the method may also be specific for and bind to wild-type HIV envelope proteins, for example, peptides that are 80% or more identical to SEQ ID NOs: 85-93.

The antibodies may be derived from a vertebrate such as a mouse, human or monkey and can be either polyclonal or monoclonal. Chimeric and humanized antibodies as well as binding fragments (e.g. Fab fragment) are also contemplated.

In yet another aspect, the invention is directed to a cell line that produces an antibody which specifically binds to one or more of SEQ ID NOs 1-84 and 94-109.

### HIV Envelope-Like Polypeptides and Polynucleotides

Certain aspects of the invention are directed to isolated HIV envelope-like polypeptides and nucleic acids encoding the polypeptides. Generally, an HIV envelope-like polypeptide of the invention has an amino acid sequence which is 80% or more identical to an amino acid sequence depicted in FIG. 1 or FIG. 16. Suitable HIV envelope-like polypeptides include both mimotope and wild-type HIV envelope polypeptides (gp160). In some embodiments, the HIV envelope-like polypeptides have an amino acid sequence which is 85%, 90%, 95%, 96%, 97%, 98%, 99% or more identical to an amino acid sequence depicted in FIG. 1 (SEQ ID NOs: 1-84), FIG. 16, or provided at SEQ ID NOs 94-109. In a preferred embodiment, the HIV envelope-like polypeptide has an amino acid sequence which is identical to an amino acid sequence depicted in FIG. 1, FIG. 16, or provided at SEQ ID NOs 94-109. HIV envelope-like peptides which are mimotopes are illustrated in FIG. 1 as SEQ ID NOs: 1-84; of these SEQ ID NOs: 21, 23, 25, 28, 29 and 31 correspond to the HIV V3 envelope region and SEQ ID NOs: 34, 35, 37, 38 and 39 correspond to the HIV envelope C-terminal region. The HIV envelope-like polypeptides may also be wild-type HIV envelope proteins, for example, peptides that are 80% or more identical to SEQ ID NOs: 85-93. In some instances, the HIV envelope-like polypeptide may be a fusion polypeptide, e.g., mimotope linked to a carrier polypeptide.

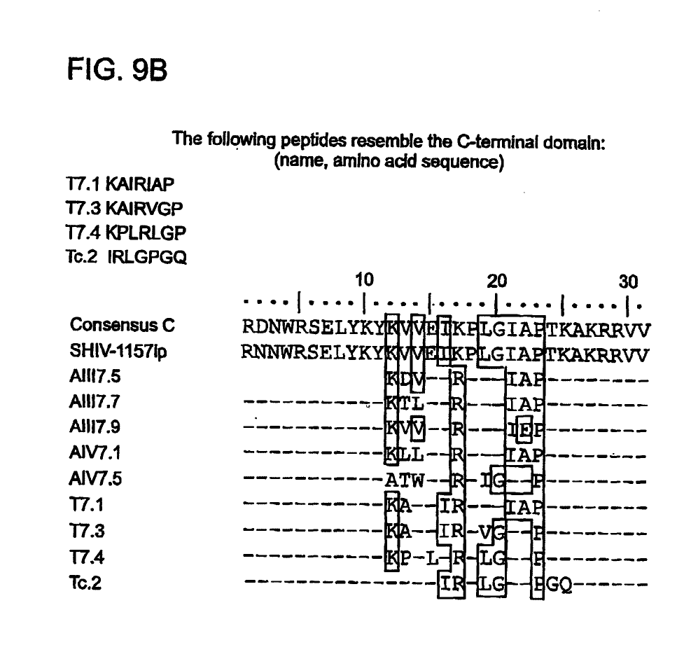

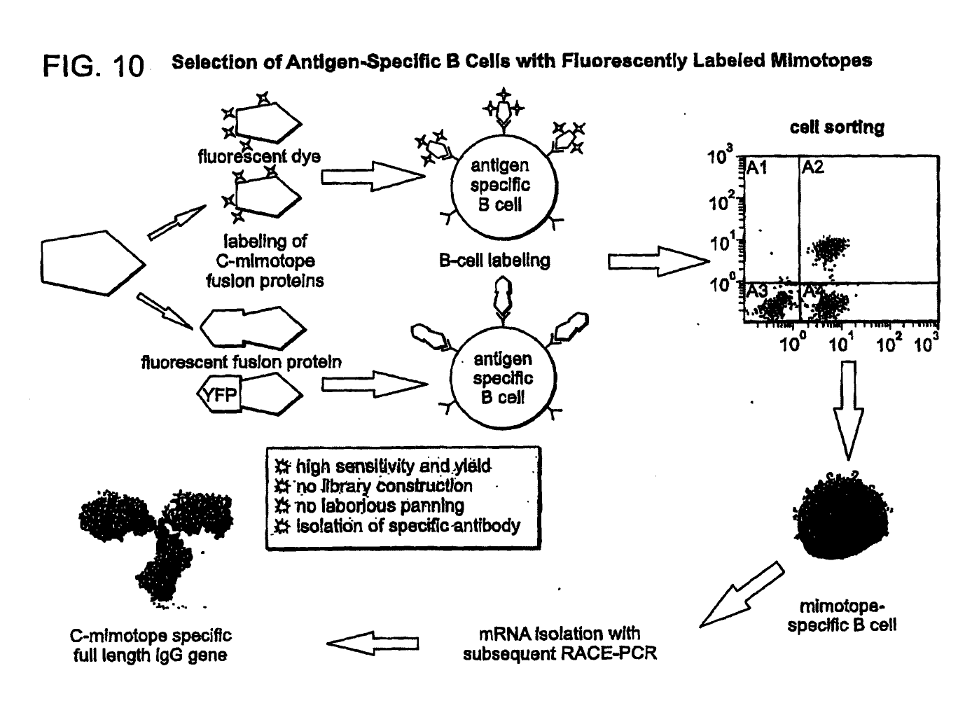

In further studies, the following peptides, which resemble the V3 loop (FIG. 9A) were identified as effective immunogens.

| | | SEQ ID NO. |
|---|---|---|
| T12.1 | TKWVHTGPGERH | 94 |
| T12.3 | MHKPIRTGPAEV | 95 |
| T12.4 | MSPPKHIRLGPN | 96 |
| T12.9 | HAKLIRTGPVAV | 97 |
| T12.5 | GLRPGRAQPFYA | 98 |
| T12.6 | MPRASPGSPHYT | 99 |
| T12.2 | TRPPPGSTAYVT | 102 |

In still other studies, the following peptides, which resemble the C-terminal domain were identified.

| Peptides | | SEQ ID NO. |
|---|---|---|
| T7.1 | KAIRIAP | 104 |
| T7.3 | KAIRVGP | 106 |
| T7.4 | KPLRLGP | 107 |
| Tc.2 | IRLGPGQ | 109 |

A polynucleotide of the present invention may be employed for producing a polypeptide by recombinant techniques. Such a polypeptide may be useful, for example, as an antigen for the induction of an immune response specific to HIV or as a reagent for the affinity purification of an antibody to HIV.

The relatively small HIV envelope-like polypeptides, i.e., up to about 50 amino acids in length, can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis. Synthesis. Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis.

The polypeptides of the present invention can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

The polypeptides of the invention can also be synthesized using recombinant techniques, well known in the art. In this regard, oligonucleotide probes can be devised based on the known sequences of HIV Env polypeptides and used to probe virus, HIV infected cells or cDNA libraries for HIV envelope-like polypeptide genes. The gene can then be further isolated using standard techniques and, e.g., restriction enzymes employed to truncate the gene at desired portions of the full-length sequence. Similarly, the HIV envelope-like peptide gene(s) can be isolated directly from cells and tissues infected with HIV, using known techniques, such as phenol extraction and the sequence further manipulated to produce the desired truncations. See, e.g., Sambrook et al., supra, for a description of techniques used to obtain and isolate DNA.

The genes encoding the HIV envelope-like peptides (e.g., full length and fragments) polypeptides can be produced synthetically, based on the amino acid sequences disclosed herein. The nucleotide sequence can be designed with the appropriate codons for the particular amino acid sequence desired. If large, the complete sequence is can be assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone a gene encoding an HIV envelope-like polypeptide which can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer which hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci. USA (1982) 79:6409.

Once coding sequences for the desired proteins have been isolated or synthesized, they can be cloned into any suitable vector or replicon for expression. A wide variety of vectors encoding modified polypeptides can be generated by creating expression constructs which operably link the polynucleotides of the invention. The construct is then introduced into a host cell for polypeptide expression. Preferably, the HIV envelope-like polypeptides are fusion polypeptides linked to an immuno-carrier protein.

Immunocarriers include polypeptides such as ankyrin repeat domains, C3d, influenza virus, albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein-derived or non-protein derived substances are known to those skilled in the art.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells).

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“FastBac” kit).

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. Following infection, cells are transfected with the DNA of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired HIV envelope-like peptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the proteins of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, a γ-interferon signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the HIV clade C envelope polypeptide substantially intact. Intracellular proteins can also be obtained by removing components from the cell wall or membrane, e.g., by the use of detergents or organic solvents, such that leakage of the Env polypeptides occurs. Such methods are known to those of skill in the art and are described in, e.g., Protein Purification Applications: A Practical Approach, (E. L. V. Harris and S. Angal, Eds., 1990).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced Env polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining an intracellular HIV envelope-like peptide of the present invention involves affinity purification, such as by immunoaffinity chromatography using anti-Env specific antibodies, or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the HIV envelope-like polypeptides can be further purified using conventional techniques well known in the art, such as by any of the techniques described above.

### Antibodies

In yet another aspect the invention is directed to an antibody that specifically binds to an HIV envelope-like polypeptide. Generally, antibodies that bind an HIV envelope-like polypeptides bind an amino acid sequence which is 80% or more identical to an amino acid sequence depicted in FIG. 1 or FIG. 16.

The present invention provides antibodies that may differentially or preferentially bind to one or more epitopes on HIV envelope. Antibodies of the invention include, but are not limited to, monoclonal antibodies, monospecific antibodies, polyclonal antibodies, multispecific antibodies, diabolizes, triabodies, tetrabodies, human antibodies, humanized antibodies, camelized antibodies, chimeric antibodies, single chain antibodies, single domain antibodies, Fab fragments, F(ab′) fragments, F(ab′)₂ fragments, Fv fragments (i.e., the smallest functional module of an antibody), single chain Fvs (scFv), disulfide-stabilized Fvs (dsFv), Fd, V_{H}, V_{L}, V_{α}, V_{β}, and anti-idiotypic (anti-Id) antibodies (including, e.g., anti-Id antibodies to antibodies of the invention), intrabodies, and epitope-binding fragments of any of the above. In some embodiments, the antibodies are monoclonal antibodies. In other embodiments, the antibodies are Fv fragments, including V_{H} and V_{L} regions.

In particular, antibodies of the present invention include immunoglobulin molecules and immunologically active portions of immunoglobulin molecules, i.e., molecules that contain an antigen binding site that binds to a HIV envelope antigen. The immunoglobulin molecules of the invention can be of any type (e.g., IgG, IgE, IgM, IgD, IgA, and IgY), class (e.g., IgG₁, IgG₂, IgG₃, IgG₄, IgA₁, and IgA₂), or subclass of immunoglobulin molecule. In a specific embodiment, an antibody of the invention is an IgG antibody. In another specific embodiment, an antibody of the invention is not an IgA antibody.

The antibodies of the invention may be from any animal origin including birds and mammals (e.g., human, mouse, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies of the present invention may be monospecific, bispecific, trispecific, or of greater multispecificity. Multispecific antibodies may be specific for different epitopes of a HIV envelope-like polypeptide. See, e.g., PCT publications WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny, et al., J Immunol 148:1547-1553 (1992).

In one embodiment, antibodies of the invention bind to cells that express HIV envelope antigens. In a specific embodiment, antibodies of the invention bind to a fragment of HIV envelope comprising 8 to 285, 8 to 275, 8 to 250, 8 to 200, 8 to 175, 8 to 150, 8 to 100, 8 to 50, 8 to 25, 15 to 50, 15 to 75, 15 to 100 or 15 to 150 amino acids of HIV envelope. Preferably, the antibodies bind to the polypeptides represented by SEQ ID NOs 1-109.

An antibody of the invention may be composed of one or two polypeptide chains. In one embodiment, an Fv antibody that binds to an HIV envelope-like polypeptide is composed of a single polypeptide chain, where the V_{H} region is bound by an amino acid linker to the V_{L} chain region. In a preferred embodiment, such scFvs are stable at 37° C. for about 2 minutes, 5 minutes, 10 minutes, 15 minutes, 20 minutes, 25 minutes, 30 minutes, 1 hour, 1.5 hours, 2 hours, 3 hours, 4 hours, 5 hours, 6 hours, 7 hours, 12 hours, 14 hours, 24 hours, 48 hours, 1 week, 2 weeks, 1 month, 3 months, 6 months, 1 year or longer as assessed by a technique known to one of skill in the art or described herein. In another embodiment, the V_{H} region of an Fv antibody that binds to 67LR is bound to a V_{L} chain region through at least one disulfide linkage (e.g., formed between respective cysteines in each chain). In certain embodiments, the disulfide linked Fv chains have a reduced tendency to aggregate as measured by, e.g., HPLC, and have a longer serum half-life. Thus, in a specific embodiment, a disulfide-stabilized Fv (dsFv) antibody comprises at least two polypeptides linked by at least one disulfide linkage. The two polypeptides can be separated by a termination codon and downstream initiation codon and ribosome binding site, so that the chains are encoded as separate open reading frames, or they can be additionally joined by a peptide linker. In order to provide disulfide covalent bonds between the V_{H} and V_{L} chains of dsFv fragments, cysteine residues are necessary. Cysteine residues can be introduced in the proper position of each V_{H} and V_{L}, determined by alignment to reference sequences, by standard molecular biology techniques (e.g., site directed mutagenesis). See Pastan, et al., U.S. Pat. No. 6,147,203, which is incorporated by reference herein in its entirety, especially columns 5-7.

In another embodiment, the V_{L} and V_{H} sequences will be followed respectively by part or all of the light and heavy chain constant regions, e.g., the whole kappa light chain constant region and the C_{H1} domain of the heavy chain constant region, with or without the heavy chain hinge domain. Thus, the genes encoding the antibody segments may occur in any order on a single plasmid, or may be expressed separately from separate plasmids. For example, in another embodiment of the invention, the V_{L} gene and any light chain constant region will be on one plasmid, while the V_{H} gene and any heavy chain constant region will be on a second plasmid. In either case, the V_{L} and/or V_{H} genes may be preceded by a signal sequence that directs the secretion of the recombinant fusion protein from the cell. See, e.g., U.S. Pat. No. 6,147,203, 6,074,644, 6,051,405 which are incorporated herein by reference in their entirety.

In another embodiment, the invention provides for single chain antibodies, in which the antibody comprises the V_{L} or V_{H} regions alone, rather than as components of Fv fragments. See, e.g., U.S. Pat. No. 6,074,644, which is incorporated herein by reference in its entirety.

An antibody can also be conjugated to a second antibody to form an antibody heteroconjugate as described by Segal in U.S. Pat. No. 4,676,980, which is incorporated herein by reference in its entirety. In a specific embodiment, two or more antibodies are cross-linked to each to produce a bispecific or multispecific antibody.

In one embodiment, antibodies with high affinity and specificity are chosen for conjugation to a cytotoxic agent, and they are then assessed for their ability to selectively and potently kill HIV or HIV infected cells.

Those skilled in the art will realize that additional modifications, deletions, insertions and the like may be made to the antibodies directed to HIV envelope-like polypeptides. Especially, deletions or other changes may be made to the antibody in order to increase stability, affinity, specificity, or, when combined with a cytotoxic agent, cytotoxicity. Typical modifications include, but are not limited to, introduction of an upstream methionine for transcription initiation, mutation of residues to cysteine in V_{H} or V_{L} regions for the creation of disulfide linkages, etc. All such constructions may be made by methods of genetic engineering well known to those skilled in the art. Fragments, analogs, and derivatives of antibodies to HIV envelope-like polypeptides can be useful in the present invention provided that when fused to a cytotoxic agent portion of the conjugate, such fragments, analogs, and derivatives maintain the ability to bind native envelope expressed on the surface of a cell or HIV particle. Preferably, the binding kinetics of the fragments, analogs, or derivatives remain the same or vary by no more than 25% (preferably, no more than 15%, 10% or 5%) as determined by an assay described herein.

To improve or alter the characteristics of antibodies to HIV envelope-like polypeptides, protein engineering may be employed. Recombinant DNA technology known to those skilled in the art can be used to create novel mutant proteins or “muteins” including single or multiple amino acid substitutions, deletions, additions, or fusion proteins. Such modified polypeptides can show, e.g., enhanced activity or increased stability. In addition, they may be purified in higher yields and show better solubility than the corresponding natural polypeptide, at least under certain purification and storage conditions. For instance, for many proteins, it is known in the art that one or more amino acids may be deleted from the amino terminus or carboxy terminus without substantial loss of biological function.

Antibodies may be altered by random mutagenesis, by error-prone PCR, random nucleotide insertion or other methods prior to recombination.

The antibodies of the invention include derivatives that are modified, i.e., by the covalent attachment of any type of molecule to the antibody such that covalent attachment does not adversely affect binding to the antigen or, in the case of antibody conjugates of the invention, adversely affect its ability to bind to HIV envelope and adversely affect its ability to kill HIV or a cell infected with HIV or inhibit transmission of HIV. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The present invention also provides antibodies that bind to HIV envelope-like polypeptides that comprise a framework region known to those of skill in the art (e.g., a human or non-human fragment). The framework region may be naturally occurring or consensus framework regions. In one embodiment, the framework region of an antibody of the invention is human (see, e.g., Clothia, et al., 1998, J Mol Biol 278:457-479 for a listing of human framework regions, which is incorporated by reference herein in its entirety).

In a specific embodiment, the present invention provides for antibodies that bind to HIV envelope-like polypeptides, said antibodies comprising the amino acid sequence of the antibodies produced by hybridoma cell lines deposited as ATCC #s and human murine framework regions. In another embodiment, the present invention provides for antibodies that bind to HIV envelope-like polypeptides, said antibodies comprising the amino acid sequence of antibodies produced by hybridoma cell lines deposited as ATCC #s and human or murine framework regions with one or more amino acid substitutions at one, two, three, or more of the following residues: (a) rare framework residues that differ between the murine antibody framework (i.e., donor antibody framework) and the human antibody framework (i.e., acceptor antibody framework); (b) Venier zone residues when differing between donor antibody framework and acceptor antibody framework; (c) interchain packing residues at the V_{H}/V_{L} interface that differ between the donor antibody framework and the acceptor antibody framework; (d) canonical residues that differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the V_{H} domain and the V_{L} domain.

The present invention also provides antibodies that bind to HIV envelope-like polypeptides that comprise constant regions known to those of skill in the art. In one embodiment, the constant regions of an antibody of the invention are human. In another embodiment, the constant regions are murine.

The present invention provides for antibodies that bind to HIV envelope-like polypeptides, the antibodies having an extended half-life in vivo and the use of such antibodies. To prolong the serum circulation of antibody conjugates (e.g., monoclonal antibodies, single chain antibodies, Fv fragments, and Fab fragments) in vivo, for example, inert polymer molecules such as high molecular weight polyethylene glycol (PEG) can be attached to the antibodies with or without a multifunctional linker either through site-specific conjugation of the PEG to the amino or carboxyl terminus of the antibodies (whichever end is not conjugated to the cytotoxic agent) or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or ion-exchange chromatography. Polyethylene glycol-derivatized antibodies can be tested for binding activity as well as for in vivo efficacy using methods well-known to those of skill in the art, for example, by immunoassays described herein.

Antibodies having an increased half-life in vivo can also be generated by introducing one or more amino acid modifications (i.e., substitutions, insertions, or deletions) into an IgG constant domain, or FcRn binding fragment thereof (preferably a Fc or hinge Fc domain fragment). See, e.g., International Publication No. WO 98/23289; International Publication No. WO 97/34631; International Publication No. WO 02/060919; and U.S. Pat. No. 6,277,375, each of which is incorporated herein by reference in its entirety.

Furthermore, antibodies can be conjugated to albumin in order to make the antibody more stable in vivo or have a longer half-life in vivo. The techniques are well-known in the art; see, e.g., International Publication Nos. WO 93/15199, WO 93/15200, and WO 01/77137; and European Patent No. EP 413,622, all of which are incorporated herein by reference.

### Methods for Producing Antibodies

Antibodies that bind to an antigen can be produced by any method known in the art for the synthesis of antibodies, in particular, by chemical synthesis or preferably, by recombinant expression techniques.

Polyclonal antibodies specific for an antigen can be produced by various procedures well-known in the art. As a non-limiting example, the antigen (i.e., HIV envelope-like polypeptides) can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Köhler & Milstein, 1975, Nature 256:495-497; Pasqualini & Arap, 2004, PNAS USA 101:257-259; Steinitz, et al., 1977, Nature 269:420-422; Vollmers, et al., 1989, Cancer Res 49:2471-2476; Vollmers & Brandlein, 2002 Hum. Antibodies 11(4):131-142; Harlow, et al., Antibodies: A Laboratory Manual, (Cold Spring Harbor Laboratory Press, 2nd ed. 1988); Hammerling, et al., in: Monoclonal Antibodies and T Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding HIV envelope-like polypeptides. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments that recognize specific HIV envelope-like polypeptides may be generated by any technique known to those of skill in the art. For example, total cellular RNA is isolated from hybridoma cells expressing the desired monoclonal antibodies using methods known to those skilled in the art, e.g., using the Qiagen RNeasy mini kit. V_{H} and V_{L} cDNAs of the monoclonal antibodies can be obtained by a RACE method using SMART RACE cDNA amplification kit (Clontech) as described in Pastan, et al., Methods Mol Biol 2004; 248:503-18, or other method for preparation of cDNA known to those of skill in the art. Prepared cDNAs are then used as the template for PCR reactions to amplify the desired V_{H} and V_{L} fragments. The PCR products can then be cloned into an appropriate vector, either directly into a vector containing the cytotoxin, or transferred to a shuttle vector, such as pCR4®-TOPO®, using the TOPO TA cloning kit (Invitrogen). The V_{H} and V_{L} chains can then be assembled into a single chain Fv (scFv) and fused to a cytotoxin as described in Pastan, et al., Methods Mol Biol 2004; 248:503-18, which is incorporated herein by reference in its entirety. As another example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH₁ domain of the heavy chain.

Further, the antibodies of the present invention can also be generated using various phage display methods known in the art. In phage display methods, functional antibody domains are displayed on the surface of phage particles that carry the polynucleotide sequences encoding them. In particular, DNA sequences encoding V_{H} and V_{L} domains are amplified from animal cDNA libraries. The DNA encoding the V_{H} and V_{L} domains are recombined together with an scFv linker by PCR and cloned into a phagemid vector. The vector is electroporated into E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman, et al., 1995, J Immunol Methods 182:41-50; Ames, et al., 1995, J Immunol Methods 184:177-186; Kettleborough, et al., 1994, Eur J Immunol 24:952-958; Persic, et al., 1997, Gene 187:9-18; Burton, et al., 1994, Advances in Immunology 57:191-280; International application No. PCT/GB91/01134; Griffiths, et al., 1994, EMBO J. 13:3245-3260; Winter, et al., 1994, Annu Rev Immunol 12:433-455; Liv, et al., 2004, Cancer Res 64:704-710; International publication Nos. WO 90/02809, WO 91/10737, WO 92/01047, WO 92/18619, WO 93/11236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax, et al., 1992, BioTechniques 12(6):864-869; Sawai, et al., 1995, AJRI 34:26-34; and Better, et al., 1988, Science 240:1041-1043 (said references incorporated by reference in their entireties).

To generate whole antibodies, PCR primers including V_{H} or V_{L} nucleotide sequences, a restriction site, and a flanking sequence to protect the restriction site can be used to amplify the V_{H} or V_{L} sequences in scFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lambda constant regions. Preferably, the vectors for expressing the V_{H} or V_{L} domains comprise an EF-1α promoter, a secretion signal, a cloning site for the variable domain, constant domains, and a selection marker such as neomycin. The V_{H} and V_{L} domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See U.S. Pat. Nos. 4,444,887 and 4,716,111; and International publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice that are incapable of expressing functional endogenous immunoglobulins, but that can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg and Huszar, 1995, Int. Rev. Immunol. 13:65 93.

For a detailed discussion of methods for producing human antibodies and human monoclonal antibodies and protocols for producing such antibodies, see, e.g., Tomizuka et al., 2000 PNAS USA 97:722-727; Davis, et al., 2004, Methods Mol Biol 248:191-200; Lagerkvist et al., 1995, Biotechniques 18:862-869; Babcook, et al., 1996 PNAS USA 93:7843-7848; International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi, et al., 1986, BioTechniques 4:214; Gillies, et al., 1989, J Immunol Methods 125:191-202; and U.S. Pat. Nos. 5,807,715, 4,816,567, 4,816,397, and 6,311,415, which are incorporated herein by reference in their entirety.

A humanized antibody is an antibody that is capable of binding to a predetermined antigen and that comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv), in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA, and IgE, and any isotype, including IgG1, IgG2, IgG3, and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. A humanized antibody can be produced using variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Pat. No. 6,407,213, U.S. Pat. No. 5,766,886, International Publication No. WO 9317105, Tan, et al., J Immunol 169:1119 25 (2002), Caldas, et al., Protein Eng 13(5):353 60 (2000), Morea, et al., Methods 20(3):267 79 (2000), Baca, et al., J Biol Chem 272(16):10678 84 (1997), Roguska, et al., Protein Eng 9(10):895 904 (1996), Couto, et al., Cancer Res 55 (23 Supp):5973s-5977s (1995), Couto, et al., Cancer Res 55(8):1717 22 (1995), Sandhu J S, Gene 150(2):409 10 (1994), and Pedersen, et al., J Mol Biol 235(3):959 73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen, et al., U.S. Pat. No. 5,585,089; and Riechmann, et al., 1988, Nature 332:323, which are incorporated herein by reference in their entireties.)

Diabodies, triabodies, and tetrabodies can be produced by techniques known to one of skill in the art. See, e.g., Kipriyanov, 2002, Methods Mol Biol 178:317-331; Todorovska, et al., 2001 J Immunol Methods 248:47-66; and Poljak, et al., 1994, Structure 2:1121-1123, each of which are incorporated herein by reference in their entirety, for methods for producing diabodies, triabodies, and tetrabodies. Single domain antibodies can also be produced by techniques known to one of skill in the art. For a description of techniques to produce single domain antibodies, see, e.g., Holliger & Hudson, 2005 Nat. Biotechnol. 23:1126-1136, Riechmann, et al., 1999, J Immunol Methods 231:25-38; and Dick, 1990, BMJ 300:659-600, each of which is incorporated herein by reference in its entirety.

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage, et al., 1999, J Mol Biol 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

### Nucleic Acids Encoding Antibodies

The invention provides nucleic acid sequences comprising a nucleotide sequence encoding an antibody or an antibody conjugate that binds to HIV envelope-like polypeptides. In a specific embodiment, such nucleic acid sequences are isolated. The invention also encompasses nucleic acid sequences that hybridize under high, intermediate, or lower stringency hybridization conditions, e.g., as defined supra, to nucleic acid sequences that encode an antibody of the invention.

The nucleic acid sequence may be obtained, and the nucleotide sequence of the nucleic acid sequence determined, by any method known in the art. A nucleic acid sequence encoding an HIV envelope-like polypeptides antibody may be generated from nucleic acid from a suitable source (e.g., the hybridoma cell lines deposited as ATCC #s).

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook, et al., 1990, Molecular Cloning, A Laboratory Manual, 2d Ed., Cold Spring Harbor Laboratory, Cold Spring Harbor, N.Y. and Ausubel, et al., eds., 1998, Current Protocols in Molecular Biology, John Wiley & Sons, NY, which are both incorporated by reference herein in their entireties), to generate antibodies having a different amino acid sequence, for example, to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia, et al., 1998, J Mol Biol 278: 457-479 for a listing of human framework regions). In a specific embodiment, one or more amino acid substitutions may be made within the framework regions, and in certain embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the nucleic acid sequence are encompassed by the present invention and within the skill of the art.

Recombinant expression of an antibody that binds to HIV envelope-like polypeptides requires construction of an expression vector containing a nucleic acid sequence that encodes the antibody. Once a nucleic acid sequence encoding an antibody molecule, heavy or light chain of an antibody, or fragment thereof (preferably, but not necessarily, containing the heavy or light chain variable domain) of the invention has been obtained, the vector for the production of the antibody molecule may be produced by recombinant DNA technology using techniques well-known in the art. Thus, methods for preparing an antibody by expressing a nucleic acid sequence encoding the antibody are described herein. Methods that are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy chain, the entire light chain, or both the entire heavy and light chains.

The expression vector is transferred to a host cell by conventional techniques and the transfected cells are then cultured by conventional techniques to produce an antibody of the invention. Thus, the invention includes host cells containing a nucleic acid sequence encoding an antibody, or a heavy or light chain thereof, or fragment thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells that may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA, or cosmid DNA expression vectors containing antibody coding sequences; yeast (e.g., Saccharomyces cerevisiae or Pichia pastoris) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecules, are used for the expression of a recombinant antibody molecule. For example, mammalian cells such as Chinese hamster ovary cells (CHO), in conjunction with a vector such as the major intermediate early gene promoter element from human cytomegalovirus, is an effective expression system for antibodies (Foecking, et al., 1986, Gene 45:101; and Cockett, et al., 1990, Bio/Technology 8:2).

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors that direct the expression of high levels of fusion protein products that are readily purified may be desirable. Such vectors include, but are not limited to, the E. coli expression vector pUR278 (Ruther, et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J Biol Chem 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of virus-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc Natl Acad Sci USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner, et al., 1987, Methods in Enzymol 153:51-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138, BT483, Hs578T, HTB2, BT2O and T47D, NS0 (a murine myeloma cell line that does not endogenously produce any immunoglobulin chains), CRL7O3O, and HsS78Bst cells.

For long-term, high-yield production of recombinant proteins, stable expression is preferred. For example, cell lines that stably express the antibody molecule may be engineered. Rather than using expression vectors that contain viral origins of replication, host cells can be transformed with DNA controlled by appropriate expression control elements (e.g., promoter, enhancer, sequences, transcription terminators, polyadenylation sites, etc.) and a selectable marker. Following the introduction of the foreign DNA, engineered cells may be allowed to grow for 1-2 days in an enriched media, and then are switched to selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the antibody molecule. Such engineered cell lines may be particularly useful in screening and evaluation of compositions that interact directly or indirectly with the antibody molecule.

A number of selection systems may be used; as non-limiting examples, the herpes simplex virus thymidine kinase (Wigler, et al., 1977, Cell 11:223), hypoxanthine-guanine phosphoribosyltransferase (Szybalska & Szybalski, 1992, Proc. Natl. Acad. Sci. USA 48:202), and adenine phosphoribosyltransferase (Lowy, et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler, et al., 1980, Proc Natl Acad Sci USA 77:357; O'Hare, et al., 1981, Proc Natl Acad Sci USA 78:1527); gpt, which confers resistance to mycophenolic acid (Mulligan & Berg, 1981, Proc Natl Acad Sci USA 78:2072); neo, which confers resistance to the aminoglycoside G-418 (Wu and Wu, 1991, Biotherapy 3:87-95; Tolstoshev, 1993, Ann Rev Pharmacol Toxicol 32:573-596; Mulligan, 1993, Science 260:926-932; and Morgan and Anderson, 1993, Ann Rev Biochem 62: 191-217; 1993, TIB TECH 11(5):155-2 15); and hygro, which confers resistance to hygromycin (Santerre, et al., 1984, Gene 30:147). Methods commonly known in the art of recombinant DNA technology may be routinely applied to select the desired recombinant clone, and such methods are described, for example, in Ausubel, et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin, et al., 1981, J Mol Biol 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse, et al., 1983, Mol Cell Biol 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc Natl Acad Sci USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies may be fused to heterologous polypeptide sequences described herein or otherwise known in the art to facilitate purification.

Once a conjugate of the invention has been produced by recombinant expression or by chemical synthesis, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

### Pharmaceutical Preparations

Vaccines

In one embodiment, an antigen of the invention is present in pharmaceutical composition. Generally antigens are in the form of a polypeptide which can be prepared by the various methods describe herein and known in the art. In some embodiments, polynucleotides encoding the antigen are provided. The polynucleotides are then transcribed and translated in vitro.

The HIV envelope-like polypeptides and the polynucleotides encoding them are useful, for example, as vaccine compositions. Vaccine compositions are used, for example, to enhance or induce an immune response of a subject to HIV. Immune responses include both humoral and cell-mediated responses. Compositions comprising particular preparations of HIV envelope-like molecules can be tested for the ability to induce an immune response using assays well known in the art. For example, a composition can be injected into a laboratory animal, such as a rat or mouse, and the animal can be monitored for the appearance of immune reactants, such as antibodies, directed against the HIV envelope-like polypeptides. Alternatively, assays such as cytotoxic T lymphocyte assays can be performed to determine whether a particular composition of HIV envelope-like molecule is immunogenic.

For use as a vaccine, a composition of the invention preferably comprises a HIV envelope-like polypeptide. Vaccine compositions of the invention typically comprise a physiologically compatible carrier. Physiologically compatible carriers are well known to those in the art and include diluents, adjuvants or excipients. Such carriers include, but are not limited to, a simple low salt solution which permits preservation of the integrity of the HIV envelope-like polypeptide, e.g., 10 mM NaCl, 0.1 mM EDTA, or to large, slowly metabolized macromolecules, such as proteins, polysaccharides, polylactic acids, polyglycolic acids, polymeric amino acids, amino acid copolymers, and inactive virus particles. Pharmaceutically acceptable salts can also be used in HIV envelope-like polypeptides compositions, for example, mineral salts such as hydrochlorides, hydrobromides, phosphates, or sulfates, as well as salts of organic acids such as acetates, proprionates, malonates, or benzoates. Compositions of the invention can also contain liquids, such as water, saline, glycerol, and ethanol, as well as substances such as wetting agents, emulsifying agents, or pH buffering agents.

Various methods can be used to administer an HIV envelope-like molecule vaccine composition to a human, including intravenous, intramuscular, or subcutaneous injection. Vaccine compositions of the invention are administered at a dose effective to induce an immune response against the HIV envelope-like polypeptide. The particular dosages of the composition used to enhance an immune response will vary, for example, according to the HIV envelope-like compositions being used and the mammal to which the composition is administered. Generally, about 5 μg to about 50 μg of HIV envelope-like polypeptide per kg of patient body weight, about 50 μg to about 5 mg/kg, about 100 μg to about 500 μg/kg, or about 200 to about 250 μg/kg will be administered to an adult human. Such ranges by no means preclude use of a higher or lower amount of a component, as might be warranted in a particular application. For example, the actual dose and schedule may vary depending on whether the compositions are administered in combination with other pharmaceutical compositions, or depending on individual differences in pharmacokinetics, drug disposition, and metabolism.

The antigens and/or adjuvants of the invention can also be delivered using one or more gene vectors, administered via nucleic acid immunization or the like using standard gene delivery protocols. Methods for gene delivery are known in the art. See, e.g., U.S. Pat. Nos. 5,399,346, 5,580,859, 5,589,466. The constructs can be delivered either subcutaneously, epidermally, intradermally, intramuscularly, intravenous, mucosally (such as nasally, rectally and vaginally), intraperitoneally, orally or combinations thereof.

An exemplary replication-deficient gene delivery vehicle that may be used in the practice of the present invention is any of the alphavirus vectors, described in, for example, U.S. Pat. Nos. 6,342,372; 6,329,201 and International Publication WO 01/92552.

A number of viral based systems have been developed for gene transfer into mammalian cells. For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109.

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476).

Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al. Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering polynucleotides, mucosally and otherwise, is the enterically administered recombinant poxvirus vaccines described by Small, Jr., P. A., et al. (U.S. Pat. No. 5,676,950, issued Oct. 14, 1997, herein incorporated by reference) as well as the vaccinia virus and avian poxviruses. By way of example, vaccinia virus recombinants expressing the genes can be constructed as follows. The DNA encoding the antigens and/or adjuvants of the invention is first inserted into an appropriate vector so that it is adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter plus the gene encoding the coding sequences of interest into the viral genome. The resulting TK recombinant can be selected by culturing the cells in the presence of 5-bromodeoxyuridine and picking viral plaques resistant thereto.

Alternatively, avipoxviruses, such as the fowlpox and canarypox viruses, can also be used to deliver genes encoding the antigens and/or adjuvants of the invention. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545. Picornavirus-derived vectors can also be used. (See, e.g., U.S. Pat. Nos. 5,614,413 and 6,063,384). Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

A vaccinia based infection/transfection system can be conveniently used to provide for inducible, transient expression of the coding sequences of interest (for example, sequences encoding the antigens or adjuvants of the invention) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase that in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

One or more additional HIV antigens can be used in the vaccine and methods of this invention. For instance, the HIV envelope-like polypeptides can be used with additional HIV antigens. The additional HIV antigens may be administered simultaneously, prior to or after administration of the HIV envelope-like polypeptides.

In addition, an immuno-modulatory factor may be added to the pharmaceutical composition. An “immuno-modulatory factor” refers to a molecule, for example a protein that is capable of modulating an immune response or DNA vectors encoding a given immuno-modulatory factor. Non-limiting examples of immunomodulatory factors include lymphokines (also known as cytokines), such as IL-6, TGF-beta, IL-1, IL-2, IL-3, IL-15, etc.); and chemokines (e.g., secreted proteins such as macrophage inhibiting factor). Certain cytokines, for example TRANCE, flt-3L, and a secreted form of CD40L are capable of enhancing the immunostimulatory capacity of APCs. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macropliage-colony stimulating factor (GM-CSF), interleukin-1 alpha (IL-1.alpha.), interleukin-11 (IL-11), MIP-1-X.gamma.MIP-β, leukemia inhibitory factor (LIF), c-kit ligand, thrombopoietin (TPO), CD40 ligand (CD40L), tumor necrosis factor-related activation-induced cytokine (TRANCE) and flt3 ligand (flt-3L). Cytokines are commercially available from several vendors such as, for example, Genzyme (Framingham, Mass.), Amgen (Thousand Oaks, Calif.), R&D Systems and Immunex (Seattle, Wash.). The sequences of many of these molecules are also available, for example, from the GenBank database. It is intended, although not always explicitly stated, that molecules having similar biological activity as wild-type or purified cytokines (e.g., recombinantly produced or mutants thereof) and nucleic acid encoding these molecules are intended to be used within the spirit and scope of the invention.

The compositions of the invention will typically be formulated with pharmaceutically acceptable carriers or diluents. As used herein, the term “pharmaceutically acceptable carrier” refers to a carrier for administration of the antigens which does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee et al. (1997) J. Microencapsul. 14(2):197-210; O'Hagan et al. (1993) Vaccine 1 (2):149-54. Such carriers are well known to those of ordinary skill in the art. Additionally, these carriers may function as immunostimulating agents (“adjuvants”). Furthermore, the antigen may be conjugated to a bacterial toxoid, such as toxoid from diphtheria, tetanus, cholera, etc., as well as toxins derived from E. coli.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of acceptable excipients is available in the well-known Remington's Pharmaceutical Sciences.

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles.

By “pharmaceutically acceptable” or “pharmacologically acceptable” is meant a material which is not biologically or otherwise undesirable, i.e., the material may be administered to an individual in a formulation or composition without causing any undesirable biological effects or interacting in a deleterious manner with any of the components of the composition in which it is contained.

Generally, the HIV envelope-like polypeptides will be operatively coupled to an immunocarrier. The vaccines of the invention additionally contemplate use of immunocarriers, such as ankyrin repeat domains, C3d, influenza virus, albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein-derived or non-protein derived substances are known to those skilled in the art.

### Antibody Pharmaceutical Preparations

The present invention provides compositions comprising an antibody of the invention. In a further embodiment, the composition includes an antibody and a carrier. The invention provides a pharmaceutical composition comprising an effective amount of an antibody of the invention and a pharmaceutically acceptable carrier or vehicle. In a specific embodiment, a pharmaceutical composition comprises an effective amount of an antibody of the invention and a pharmaceutical acceptable carrier or vehicle. The pharmaceutical compositions are suitable for veterinary and/or human administration.

The pharmaceutical compositions of the present invention can be in any form that allows for the composition to be administered to a subject, said subject preferably being an animal, including, but not limited to a human, mammal, or non-human animal, such as a monkey, cow, horse, sheep, pig, fowl, cat, dog, mouse, rat, rabbit, guinea pig, etc., and is more preferably a mammal, and most preferably a human.

The antibody compositions of the invention can be in the form of a solid, liquid or gas (aerosol). Preferably the composition is in a liquid pharmaceutical composition. Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection or infusion techniques. In a specific embodiment, the compositions are administered parenterally. In a more specific embodiment, the compositions are administered intravenously. Pharmaceutical compositions of the invention can be formulated so as to allow an antibody of the invention to be bioavailable upon administration of the composition to a subject. Compositions can take the form of one or more dosage units, where, for example, a tablet can be a single dosage unit, and a container of an antibody of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of cancer the subject is in need of treatment of, the use of the composition as part of a multi-drug regimen, the particular form of the antibody of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

Physiologically compatible carriers are well known to those in the art and include diluents, adjuvants or excipients. Such carriers include, but are not limited to, a simple low salt solution which permits preservation of the integrity of the HIV envelope-like polypeptide, e.g., 10 mM NaCl, 0.1 mM EDTA, or to large, slowly metabolized macromolecules, such as proteins, polysaccharides, polylactic acids, polyglycolic acids, polymeric amino acids, amino acid copolymers, and inactive virus particles. Pharmaceutically acceptable salts can also be used in HIV envelope-like polypeptides compositions, for example, mineral salts such as hydrochlorides, hydrobromides, phosphates, or sulfates, as well as salts of organic acids such as acetates, proprionates, malonates, or benzoates. Compositions of the invention can also contain liquids, such as water, saline, glycerol, and ethanol, as well as substances such as wetting agents, emulsifying agents, or pH buffering agents. The antibody pharmaceutical composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The antibody liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant. An injectable composition is preferably sterile.

The pharmaceutical compositions comprise an effective amount of an antibody of the invention such that a suitable dosage will be obtained (see infra, for suitable dosages). Typically, this amount is at least 0.01% of an antibody of the invention by weight of the composition. When intended for oral administration, this amount can be varied to be between 0.1% and 80% by weight of the composition. Preferred oral compositions can comprise from between 4% and 50% of the antibody of the invention by weight of the composition. Preferred compositions of the present invention are prepared so that a parenteral dosage unit contains from between 0.01% and 2% by weight of the antibody of the invention.

The compositions of the invention can be administered by any convenient route, for example, by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering an antibody of the invention. In certain embodiments, more than one antibody of the invention is administered to a subject. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. The preferred mode of administration is left to the discretion of the practitioner, and will depend in-part upon the site of the medical condition (such as the site of cancer, a cancerous tumor or a pre-cancerous condition).

In one embodiment, the antibodies or antibody conjugates of the invention are administered parenterally. In a specific embodiment, the antibodies or antibody conjugates of the invention are administered intravenously.

In yet another embodiment, the antibody or antibody conjugates of the invention can be delivered in a controlled release system. In one embodiment, a pump can be used (see Sefton, CRC Crit. Ref Biomed Eng 1987, 14, 201; Buchwald, et al., Surgery 1980, 88: 507; Saudek, et al., N Engl J Med 1989, 321: 574). In another embodiment, polymeric materials can be used (see Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J Macromol Sci Rev Macromol Chem 1983, 23, 61; see also Levy, et al., Science 1985, 228, 190; During, et al., Ann Neurol, 1989, 25, 351; Howard, et al., J Neurosurg, 1989, 71, 105). Other controlled-release systems discussed in the review by Langer (Science 1990, 249, 1527-1533) can be used.

In another embodiment, polymeric materials can be used to achieve controlled or sustained release of the antibodies or antibody conjugates of the invention (see, e.g., U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in Remington's Pharmaceutical Sciences by E. W. Martin.

Sustained or directed release compositions that can be formulated include, but are not limited to, antibodies or antibody conjugates of the invention protected with differentially degradable coatings, e.g., by microencapsulation, multiple coatings. It is also possible to freeze-dry the compositions and use the lyophilizates obtained, for example, for the preparation of products for injection.

In a preferred embodiment, the antibodies or antibody conjugates of the invention are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetics such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where an antibody of the invention is to be administered by infusion, it can be dispensed, for example, with an infusion bottle containing sterile pharmaceutical grade water or saline. Where the antibody of the invention is administered by injection, an ampoule of sterile water for injection or saline can be provided so that the ingredients can be mixed prior to administration.

The pharmaceutical compositions of the invention can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a concentration of a conjugate of the invention of from between 0.01% and 10% w/v (weight per unit volume of composition).

The compositions can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

Whether in solid, liquid or gaseous form, the compositions of the present invention can comprise an additional active agent selected from among those including, but not limited to, an additional therapeutic agent, an antiemetic agent, a hematopoietic colony stimulating factor, an adjuvant therapy, a vaccine or other immune stimulating agent, an antibody/antibody fragment-based agent, an anti-depressant and an analgesic agent. For instance in a particular embodiment, the pharmaceutical composition comprises a conjugate of the invention, an additional agent, and a pharmaceutically acceptable carrier or vehicle.

The pharmaceutical compositions can be prepared using methodology well known in the pharmaceutical art. For example, a composition intended to be administered by injection can be prepared by combining a conjugate of the invention with water so as to form a solution. A surfactant can be added to facilitate the formation of a homogeneous solution or suspension. Surfactants are complexes that can non-covalently interact with a conjugate of the invention so as to facilitate dissolution or homogeneous suspension of the conjugate of the invention in the aqueous delivery system.

In one embodiment, the pharmaceutical compositions of the present invention may comprise one or more other therapies.

### Methods of Use

The invention is also directed to methods for inducing an immune response in a subject by administering a pharmaceutical composition having an immunologically effective dose of one or more HIV envelope-like polypeptides. Administration can occur before or during an HIV infection. Generally, the HIV envelope-like polypeptide is 80% or more identical to an amino acid sequence of FIG. 1 or FIG. 16. Suitable HIV envelope-like polypeptides include both mimotope and wild-type HIV envelope polypeptides (gp160). In some embodiments, the HIV envelope-like polypeptide has an amino acid sequence which is 85%, 90%, 95%, 96%, 97%, 98%, 99% or more identical to an amino acid sequence depicted in FIG. 1 or FIG. 16. In a preferred embodiment, the subject is administered one or more envelope-like polypeptides having an amino acid sequence which is identical to an amino acid sequence depicted in FIG. 1 or FIG. 16. Peptides suitable for inducing a protective immune response include HIV envelope-like peptides having the amino acid sequence of SEQ ID NOs: 1-84, of these SEQ ID NOs: 21, 23, 25, 28, 29 and 31 correspond to HIV V3 envelope region SEQ ID NOs: 34, 35, 37, 38 and 39 correspond to the HIV envelope C-terminal region. Alternatively, the HIV envelope-like polypeptides included in the pharmaceutical composition may be wild-type HIV envelope proteins, for example, peptides that are 80% or more identical to SEQ ID NOs: 85-93. Preferably, the HIV envelope-like polypeptides are fusion polypeptide, e.g., mimotope-carrier fusion polypeptide. In some embodiments, the method of inducing an immune response includes the sequential or co-administration of a nucleic acid encoding a HIV envelope polypeptide (e.g., gp160).

Administering a therapeutically effective dose of an antibody that specifically binds to an HIV envelope-like polypeptide may also be used as a means for treating or preventing an HIV infection in a subject. Administration can occur before or after HIV infection. Generally, the HIV envelope like polypeptide has an amino acid sequence which is 80% or more identical to an amino acid sequence depicted in FIG. 1 or FIG. 16. Suitable antibodies bind to HIV envelope-like polypeptides include both mimotope and wild-type HIV envelope polypeptides (gp160). In some embodiments, the antibodies bind to HIV envelope-like polypeptides having an amino acid sequence which is 85%, 90%, 95%, 96%, 97%, 98%, 99% or more identical to an amino acid sequence depicted in FIG. 1 or FIG. 16. In a preferred embodiment, the antibody binds to an envelope-like polypeptide having an amino acid sequence which is identical to an amino acid sequence depicted in FIG. 1 or FIG. 16. In one embodiment, the antibody binds to a mimotope of an HIV envelope peptide such as those illustrated in SEQ ID NOs: 1-84 and 94-109. More specifically the antibody binds to a mimotope of the HIV V3 envelope region, wherein the mimotope has an amino acid sequence which is 80% or more identical to SEQ ID NOs: 21, 23, 25, 28, 29, 31, and 94-103. In another embodiment, the antibody used in the method of treatment is a mimotope derived from the HIV C-terminal envelope region and has an amino acid sequence which is 80% or more identical to the amino acid sequence of SEQ ID NOs: 34, 35, 37, 38, 39, and 104-109. The antibodies used in the method may also be specific for and bind to wild-type HIV envelope proteins, for example, peptides that are 80% or more identical to SEQ ID NOs: 85-93.

Some terms relating to the use of the antigens and/or antibodies of this invention are defined as follows.

As used herein, the term “inducing an immune response” to an antigen or composition is the development in a subject of a humoral and/or a cellular immune response to an antigen present in the composition of interest.

The terms “treat,” and “treating,” as used herein, shall mean decrease, suppress, attenuate, diminish, arrest, or stabilize the development or progression of HIV or the symptoms associated with HIV (e.g., AIDS) in an animal who is infected with the viral disorder. The treatment may be complete, e.g., the total absence of HIV in a subject. The treatment may also be partial, such that the occurrence of infected cells in a subject is less than that which would have occurred without the present invention. Treatment results in the stabilization, reduction or elimination of the infected cells, an increase in the survival of the patient or a decrease of the symptoms of the disease.

The terms “prevent,” “preventing,” and “prevention,” as used herein, shall refer to a decrease in the occurrence of HIV or decrease in the risk of acquiring HIV or its associated symptoms in a subject (e.g., AIDS). The prevention may be complete, e.g., the total absence of disease or pathological cells in a subject. The prevention may also be partial, such that the occurrence of the disease or pathological cells in a subject is less than that which would have occurred without the present invention.

The term “inhibits” as used herein with reference to an HIV infection refers to a decrease in viral transmission, decrease in virus binding to a cellular target or decrease in disease or a symptom thereof. For example, the polypeptides of the present invention induce an antibody response that is used to inhibit viral transmission, syncytium formation and disease associated with the virus (e.g. AIDS). A compound of the invention can be screened by any assay known in the art and described herein, to determine whether the compound prevents or treats an HIV infection, or otherwise inhibits the virus (e.g., reduces infectivity, transmission). For example, an antibody of the invention can be assayed for its ability to inhibit viral infectivity by contacting a cell culture that is incubated with the virus with a test compound. The antibody is found to inhibit viral infectivity when viral infectivity is 90%, 80%, 75%, 70%, 60%, 50%, 40%, 30%, 20%, 10%, or 5% or less in the presence of the test antibody as compared to a suitable control (subjected to an unspecific control-antibody).

The term “inhibit transmission”, as used herein, refers to the antibody's ability to inhibit viral infection of cells, via, for example, cell—cell fusion or cell-free virus infection. Such infection may involve membrane fusion, as occurs in the case of enveloped viruses, or some other fusion event involving a viral structure and a cellular structure.

The term “inhibiting syncytium formation”, as used herein, refers to an antibody's ability to inhibit or reduce the level of membrane fusion events between two or more moieties relative to the level of membrane fusion which occurs between said moieties in the absence of the agent. The moieties may be, for example, cell membranes or viral structures, such as viral envelopes.

The terms “therapeutically effective amount,” or “therapeutically effective dose” refers to that amount of an agent (e.g., polypeptide or antibody) that produces an intended pharmacological, therapeutic or preventive result. The pharmacologically effective amount results in the amelioration of one or more symptoms of a viral disorder, or prevents the advancement of a viral disease, or causes the regression of the disease or decreases viral transmission. For example, a therapeutically effective amount preferably refers to the amount of a therapeutic compound (e.g., antibody) that decreases the rate of transmission, decreases HIV viral load, or decreases the number of infected cells, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more. A therapeutically effective amount, with reference to HIV, also refers to the amount of a therapeutic agent (e.g., antibody) that increases CD4⁺ cell counts, increases time to progression to AIDS, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more.

The compositions will include “immunologically effective amounts” of HIV envelope-like antigen i.e. amounts sufficient to raise a specific immune response or, more preferably, to treat, reduce, or prevent HIV infection. An immune response can be detected by looking for antibodies to the HIV envelope-like antigen used (e.g. IgG or IgA) in patient samples (e.g. in blood or serum, in mesenteric lymph nodes, in spleen, in gastric mucosa, and/or in feces). The precise effective amount for a given patient will depend upon the patient's age, size, health, the nature and extent of the condition, the precise composition selected for administration, the patient's taxonomic group, the capacity of the patient's immune system to synthesize antibodies, the degree of protection desired, the formulation of the vaccine, the treating physician's assessment of the medical situation, and other relevant factors. Thus, it is not useful to specify an exact effective amount in advance, but the amount will fall in a relatively broad range that can be determined through routine trials, and is within the judgment of the clinician. For purposes of the present invention, an effective dose will typically be from about 0.01 mg/kg to 50 mg/kg in the individual to which it is administered. Preferably, the immune response is an antibody response which is specific for an HIV envelope polypeptide and inhibits an HIV infection.

The antigens (e.g., HIV envelope-like polypeptide) of this invention are expected to be valuable as vaccine immunogens due to their enhanced immunogenicity, enhanced stability and half-life, and their ability to elicit effective neutralizing antibodies that are broadly cross-reactive against a spectrum of HIV isolates and do not react with self-antigens (unlike antibodies elicited by many known gp41-based antigens). Accordingly, an embodiment of this invention includes a method of vaccinating against HIV infections in a subject comprising administering to said subject a pharmaceutical composition containing an amount of an antigen of the invention that is effective in immunizing (at least partially) against HIV infection.

The determination of a therapeutically effective dose is well within the capability of those skilled in the art. A therapeutically effective dose refers to the amount of an antibody that may be used to effectively treat a disease (e.g., cancer) compared with the efficacy that is evident in the absence of the therapeutically effective dose.

The therapeutically effective dose may be estimated initially in animal models (e.g., rats, mice, rabbits, dogs, or pigs). The animal model may also be used to determine the appropriate concentration range and route of administration. Such information may then be used to determine useful doses and routes for administration in humans.

The antigens and/or antibodies of the present invention may be administered alone or in combination with one or more additional therapeutic agents. Combination therapy includes administration of a single pharmaceutical dosage formulation which contains an antibody of the present invention and one or more additional therapeutic agents, as well as administration of the antibody of the present invention and each additional therapeutic agent in its own separate pharmaceutical dosage formulation. For example, an antibody of the present invention and a therapeutic agent may be administered to the patient together in a single intravenous composition or each agent may be administered in separate intravenous formulations.

Where separate dosage formulations are used, the antibody of the present invention and one or more additional therapeutic agents may be administered at essentially the same time (e.g., concurrently) or at separately staggered times (e.g., sequentially). The order of administration of the agents is not limited.

For example, in one aspect, co-administration of an antibody or antibody fragment of the invention together with one or more anti-HIV agents to potentiate the effect of either the antibody or the anti-HIV agent(s) or both is contemplated for use in treating HIV infections. Examples of anti-HIV agents include, but are not limited to AGENERASE (ampreavir), APTIVUS (tipranavir), ATRIPLA, COMBIVIR, RETROVIR, EPIVIR, CRIXIVAN (indinavir), EMTRIVA (emtricitabine), EPZICOM, FORTOVASE (saquinavir), FUZEON (enfuvirtide), HIVID (ddc/zalcitabine), INTELENCE (Etravirine), ISENTRESS (raltegravir), INVIRASE (saquinavir), KAETRA (lopinavir), LEXIVA (Fosamprenavir), NORVIR (ritonavir), PREZISTA (darunavir), RESCRTIPTOR (delavirdine), RETROVIR (AZT), REYATAZ (atazanavir), SUSTIVA (efavirenz), TRIZIVIR, VIDEX (ddl/didanosine), VIRACEPT (nelfinavir), VIRAMUNE (nevirapine), VIREAD (tenofovir disoproxil fumarate), ZERIT (d4t/stavudine) and ZIAGEN (abacavir).

Therapeutic efficacy and toxicity (e.g., ED₅₀—the dose therapeutically effective in 50% of the population and LD₅₀—the dose lethal to 50% of the population) of an antibody may be determined by standard pharmaceutical procedures in cell cultures or experimental animals. The dose ratio of toxic to therapeutic effects is the therapeutic index, and it may be expressed as the ratio, LD₅₀/ED₅₀. The data obtained from animal studies may used in formulating a range of dosage for human use. The dosage contained in such compositions may be within a range of circulating concentrations that include the ED₅₀ with little or no toxicity. The dosage varies within this range depending upon the dosage form employed, sensitivity of the patient, and the route of administration.

The exact dosage may be determined by the practitioner, in light of factors related to the patient who requires treatment. Dosage and administration may be adjusted to provide sufficient levels of the antibody or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy.

Effective in vivo dosages of an antigen and/or antibody are in the range of about 5 μg to about 500 μg/kg of patient body weight. For administration of polynucleotides encoding the antibodies, effective in vivo dosages are in the range of about 100 ng to about 500 μg of DNA.

The antibodies of the invention may be used as therapeutics in the treatment of HIV infection and/or AIDS. In addition, the antibodies may be used as prophylactic measures in previously uninfected individuals after acute exposure to an HIV virus (e.g. post-exposure prophylaxis). Examples of such prophylactic use of the antibodies may include, but are not limited to, prevention of virus transmission from mother to infant and other settings where the likelihood of HIV transmission exists, such as, for example, sexual transmission or accidents in health care settings wherein workers are exposed to HIV-containing blood products.

Effective dosages of the compositions of the invention to be administered may be determined through procedures described herein and well known to those in the art which address such parameters as biological half-life, bioavailability, and toxicity.

### Assaying for Anti-HIV Activity

Described herein, are methods for evaluating the ability of a compound, such as the antibodies of the invention, to prevent, treat, or otherwise inhibit an HIV infection or HIV activity, wither in vitro or in vivo. Specifically, such assays are described below and in Examples 1 and 5. Additional assays for evaluating anti-vial activity are well known to those with ordinary skill in the art.

The antiviral activity exhibited by the antibodies of the invention may be measured, for example, by easily performed in vitro assays, such as those described herein and known by those of ordinary skill in the art, which can test the antibody's ability to inhibit syncytia formation, or their ability to inhibit infection by cell-free virus (Madani, N., et al., Journal of Virology, 2007. 81(2): p. 532-538; Si, Z. H., M. Cayabyab, and J. Sodroski, Journal of Virology, 2001. 75(9): p. 4208-4218; Si, Z. H., et al., PNAS USA, 2004. 101(14): p. 5036-5041).

Using these assays, such parameters as the relative antiviral activity of the antibodies exhibit against a given strain of virus and/or the strain specific inhibitory activity of the peptide can be determined.

Assays to test an antibody's antiviral capabilities are contemplated with the present invention. For example, a reverse transcriptase (RT) assay may be utilized to test the antibody's ability to inhibit infection of CD4⁺ cells by cell-free HIV. Such an assay may comprise culturing an appropriate concentration (i.e., Tissue Culture Infectious Dose 50) of virus and CD4⁺ cells in the presence of the antibody's to be tested. Culture conditions well known to those in the art are used. A range of antibody concentrations may be used, in addition to a control culture wherein no antibody has been added. After incubation for an appropriate period (e.g., 7 days) of culturing, a cell-free supernatant is prepared, using standard procedures, and tested for the present of RT activity as a measure of successful infection. The RT activity may be tested using standard techniques such as those described by, for example, Goff et al. (Goff, S. et al., 1981, J. Virol. 38:239-248) and/or Willey et al. (Willey, R. et al., 1988, J. Virol. 62:139-147). These references are incorporated herein by reference in their entirety.

In vivo assays may also be utilized to test, for example, the antiviral activity of the antibodies of the invention. To test for anti-HIV activity, for example, the in vivo model described in Barnett et al. (Barnett, S. W. et al., 1994, Science 266:642-646) may be used.

Additional assays for evaluating the effectiveness of the modified viral polypeptides are well known to those of ordinary skill in the art.

### Kits

The present invention also encompasses a finished packaged and labeled pharmaceutical product. This article of manufacture includes the appropriate unit dosage form in an appropriate vessel or container such as a glass vial or other container that is hermetically sealed. The pharmaceutical product may contain, for example, a compound of the invention (e.g., HIV envelope-like polypeptide, antibody) in a unit dosage form in a first container, and in a second container, sterile water for injection. Alternatively, the unit dosage form may be a solid suitable for oral, transdermal, intranasal, intravaginal, cervical ring, or topical delivery.

In a specific embodiment, the unit dosage form is suitable for intravenous, intramuscular, intranasal, oral, intravaginal, cervical, topical or subcutaneous delivery. Thus, the invention encompasses solutions, solids, foams, gels, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (e.g. detection and quantitation of infection), and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a compound of the invention, and wherein said packaging material includes instruction means which indicate that said compound can be used to prevent, manage, treat, and/or ameliorate one or more symptoms associated with a viral disease by administering specific doses and using specific dosing regimens as described herein.

The following examples are provided merely as illustrative of various aspects of the invention and shall not be construed to limit the invention in any way.

### EXAMPLES

### Example 1

### Materials and Methods for Practicing Examples 2-7

### Monkey Serum.

Serum was collected from rhesus monkey RK1-8, an animal described earlier (Song R J, et al. (2006). J Virol 80: 8729-8738). Briefly, this monkey was infected with a pathogenic R5SHIV, termed SHIV-1157ip, which encodes the env gene of HIV-C, the most prevalent HIV-1 subtype worldwide. Approximately 10% of the rhesus monkeys infected with SHIV-1157ip or the related SHIV-1157ipd3N4 developed high-titer, broadly reactive nAbs not only against homologous SHIV-1157ip and the corresponding primary HIV-C, but also against heterologous strains of HIV-1 clades B and C (Table 1 paper (p. 42, Humbert et al., PLOS One submitted)).

### Phage Display Biopanning.

Paramagnetic beads (Dynabeads M-280 tosyl activated; Invitrogen, Carlsbad Calif., USA) were coated with a rabbit anti-monkey IgG (Sigma-Aldrich, St. Louis Mo., USA) according to the manufacturer's instructions. Coated beads were pre-incubated while rotating for 2 h at room temperature with rhesus monkey serum (1:250 in phosphate buffered saline/0.25% gelatin, PBSG; Gibco-Invitrogen, Grand Island N.Y., USA; Fisher Scientific, Fair Lawn N.J., USA). Beads were washed 5× with PBSG/0.5% (w/v) Tween-20 (PBSGT; Sigma-Aldrich) and then incubated while rotating overnight at 4° C. with 10 μl of the original phage-displayed peptide library (New England Biolabs, Ipswich Mass., USA). Biopannings of all three libraries (7mer, cyclic 7mer and 12mer) were performed in parallel using separate tubes. The next day, beads were washed 10× with 1 ml PBSGT and bound phages were eluted by pH shift with 0.2 M glycine-HCl pH 2.2 supplemented with 1 mg/ml BSA (Sigma-Aldrich). After neutralization with 1 M Tris-HCl pH 9.1 (Sigma-Aldrich), eluted phages were subjected to negative selection using pooled sera from non-infected control monkeys. Phages remaining from the negative selections were amplified in Escherichia coli (ER2738, New England Biolabs), precipitated overnight at 4° C. (20% PEG-8000/2.5 M NaCl; Fisher Scientific) and used for a second and third round of selection. After the third positive selection, the phages were titered, and single clones were picked and tested by phage ELISA for specific binding. Positives clones were amplified and sequenced to deduce their peptide insert.

### Phage ELISA.

Plates (Greiner-Bio-One GmbH, Frickenhausen, Germany) were coated overnight at 4° C. with 100 μl/well serum (1:5,000 in carbonate-bicarbonate buffer; Sigma-Aldrich). The next day, plates were blocked (1 h, room temperature) with 200 μl/well 3% casein (Sigma-Aldrich) in PBS/0.5% Tween-20 (PBSCT) and washed 3× with 300 μl dH₂O in an automated plate washer (BioTek Instruments, Inc., Winooski Vt., USA). Then, 70 μl of control or positively selected phages that had been amplified overnight were added to 30 μl PBSCT and incubated overnight at 4° C. Plates were washed 3× and incubated for 1 h at RT with 100 μl/well of an anti-phage HRP-conjugated antibody (1:2,000 in PBSCT; GE Healthcare Bio-Sciences Corp., Piscataway N.J., USA). After washing 5×, the plates were developed with 100 μl/well o-phenylenediamine in phosphate-citrate buffer (Sigma-Aldrich), stopped with 100 μl/well 1 NH₂SO₄ (VWR, West Chester Pa., USA) and read at 490/620 nm.

### Mimotope Analysis.

Phage insert sequences were analyzed using the computer program 3DEX (Schreiber A, et al. (2005). J Comput Chem 26: 879-887). After checking for linear homology to SHIV-1157ip gp160 (Song R J, et al. (2006). J Virol 80: 8729-8738), the peptide sequences were compared to published PDB structure files of HIV-1 gp120 (Kwong P D, et al. (2000). Structure 8: 1329-1339, Huang C C, et al. (2005). Science 310: 1025-1028) to identify conformational homology. Phages were grouped according to their peptide motifs, and selected phages were used for further analysis and immunization studies (FIG. 2).

### Immunization of Mice.

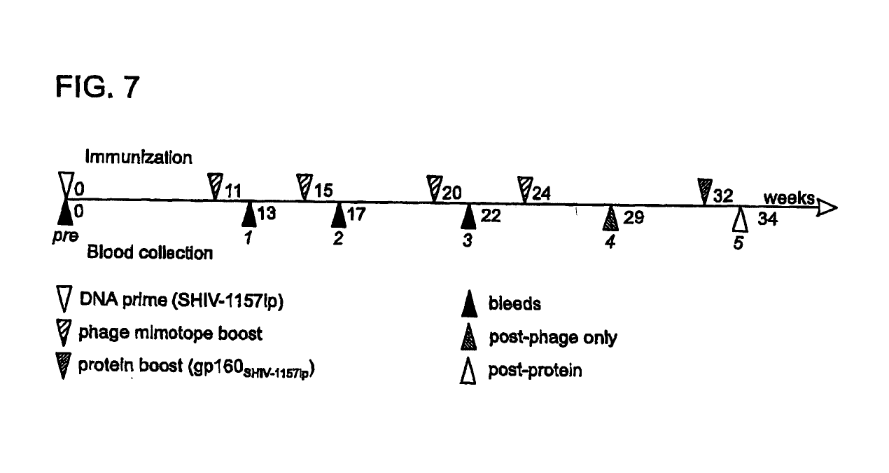

Selected recombinant phages were grouped according to motifs; each group consisted of 5-6 phages with similar but not identical peptide sequences. Phages of each group were combined and used as five different mixtures for immunization. Mice were primed once with SHIV-1157ip env DNA (intramuscularly; 100 μg in 100 μl PBS) and boosted subcutaneously (s.c.) with 10¹² phage particles in 100 μl PBS/MPL (Sigma-Aldrich) every 4-5 weeks. Serum samples were collected 2-5 weeks after each boost. After four phage boosts, serum samples were tested for their neutralizing capacity against HIV-1_{SG162.LS} and SHIV-1157ip. In a pilot study, 11 mice were given an additional boost with trimeric SHIV-1157ip gp160 (s.c.; 20 μg in 100 μl PBS with incomplete Freund's adjuvant (IFA) (Sigma-Aldrich) and their sera were tested for the presence of neutralizing antibodies (FIG. 7).

### Neutralization Assay.

SHIV-1175ip was prepared in rhesus monkey PBMC, HIV pIndieC was prepared in human PBMC and HIV_{SF162.LS} pseudovirus was prepared using cotransfection of 293T cells with an env expression plasmid and Δenv backbone vector. TZM-b1 cells encode the luciferase gene under the control of the HIV-1 promoter; both CD4 and CCR5 are also expressed on the cell surface (AIDS Research and Reference Program, Division of AIDS, NIAID, NIH). A total of 5000 cells/well were seeded overnight in 100 μl DMEM/10% FCS (Gibco-Invitrogen, Grand Island N.Y., USA). Serial 2-fold dilutions of immune sera were prepared in triplicates in 96-well round-bottom plates (Becton Dickenson, Franklin Lakes N.J., USA). In parallel, the pre-immune sera were serially diluted and used as controls. “Virus only” wells received 50 μl medium. The virus was diluted (1:500 for SHIV-1157ip: 36 ng/ml p27; 1:500 for HIV pIndieC: 35 ng/ml p24; 1:300 for HIV_{SF162.LS}: 300 ng/ml p24) and 50 μl of virus was added to all wells. The plate was incubated for 1 h at 37° C. in 5% CO₂, after which time 10 μl of a 400 μg/ml DEAE-Dextran solution (Sigma-Aldrich) was added to all wells and the entire mixture was transferred into the 96-well flat-bottom plate with the seeded TZM-b1 cells. The next day, medium was replaced with fresh medium and incubated another 24 h. Bright-Glo luciferase substrate (Promega, Madison Wis., USA) was added to the plate the following day and luciferase activity was measured. The percent neutralization was calculated using the following equation:

$$\% Neutralization = [ 1 - ( \frac{Luciferase immune serum}{Luciferase pre \text{-} immune serum} ) ] * 100$$

### Dot Spot Analysis.

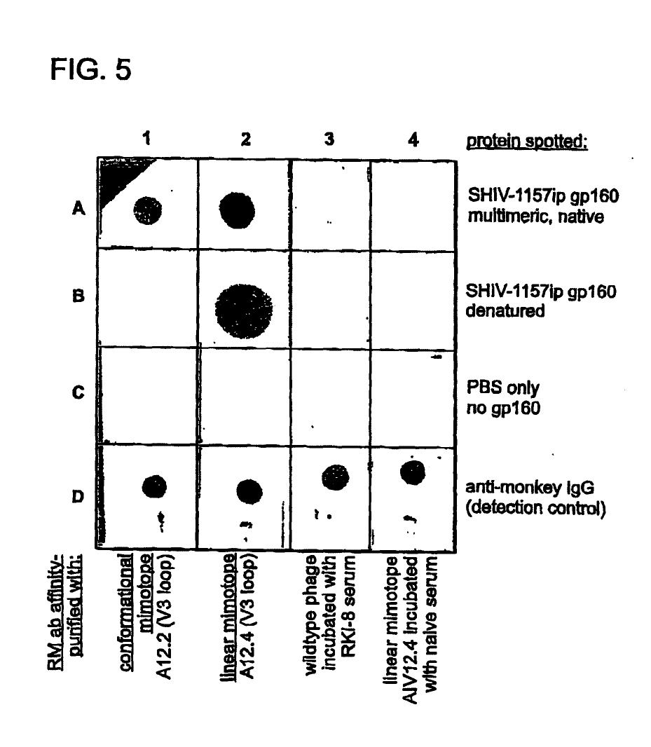

Trimeric SHIV-1157 gp160 was spotted onto a nitrocellulose membrane (Whatman GmbH, Dassel, Germany) (50 ng/spot in native or reduced conditions (10 mM TCEP (Pierce, Rockford Ill., USA); 1% SDS (Sigma-Aldrich); boiled for 2 min). Strips were blocked with PBSCT for 1 h at room temperature and incubated overnight at 4° C. with antibodies (diluted in PBSCT). The next day, strips were washed 3× with PBST and incubated with anti-monkey IgG horse-radish peroxidase (HRP)-conjugate (1:2,000 in PBSCT; Sigma-Aldrich). After 1 h at room temperature, strips were washed 5× with PBST and developed using Opti-4CN substrate (Bio-Rad Laboratories, Hercules Calif., USA) (FIG. 5).

### ELISA.

Plates were coated overnight at 4° C. with 100 ng/well native or reduced (10 mM TCEP/1% SDS; boiled for 2 min) proteins in 100 μl coating buffer. After washing 3×, plates were blocked with 200 μl/well PBSCT for 1 h at room temperature and then incubated overnight at 4° C. with mouse serum (1:175) or rhesus monkey serum (1:800; 100 μl/well diluted in PBSCT). Plates were washed 3× and incubated with horse-radish peroxidase (HRP)-conjugated antibodies (1:2,000 in PBSCT, 100 μl/well) for 1 h at room temperature. After washing 5×, plates were developed using 100 μl/well o-phenylenediamine in phosphate-citrate buffer, stopped with 100 μl/well 1 NH₂SO₄ and read at 490/620 nm.

### Mimotope Fusion Proteins.

Fusion proteins were cloned into the expression vector pPEPTIDE according to manufacturer's instructions (MoBiTec GmbH, Gottingen, Germany). Briefly, phage peptide insert were amplified using two primers (pPeptide-rev: 5′-GGCCCGGGGATCCTAACTTTCAACAGTTTCGGCCGAACCTCCACC; pPeptide-fw: 5′-CGCCCGCGGATTAATGGCCCTTTAGTGGTACCTTTCTATTCTCACTCT) to introduce the underlined cloning restriction sites, digested with Asel and BamHI (Fermentas Inc., Glen Burnie Md. USA) and gel-purified using NucleoSpin Extract II (Macherey-Nagel Inc., Bethlehem Pa., USA). Six ng of mimotope DNA were ligated with 100 ng vector and transformed into BL21(DE3) cells (Novagen, Madison Wis., USA). After sequence analysis, proteins were expressed according to the manual and purified using standard chromatography using the Ni-charged Profinity IMAC resin (Bio-Rad). Of note, the vector encodes a His tag to assess fusion protein expression. The plasmid is based on the pET expression vector system and induction of the T7 promoter leads to the expression of a highly expressed fusion protein (89 aa) followed by a higher affinity poly(His) region (53 aa) for purification and the C-terminal mimotope. The expressed mimotope fusion protein migrates at a size of around 26 kD.

### Western Blot.

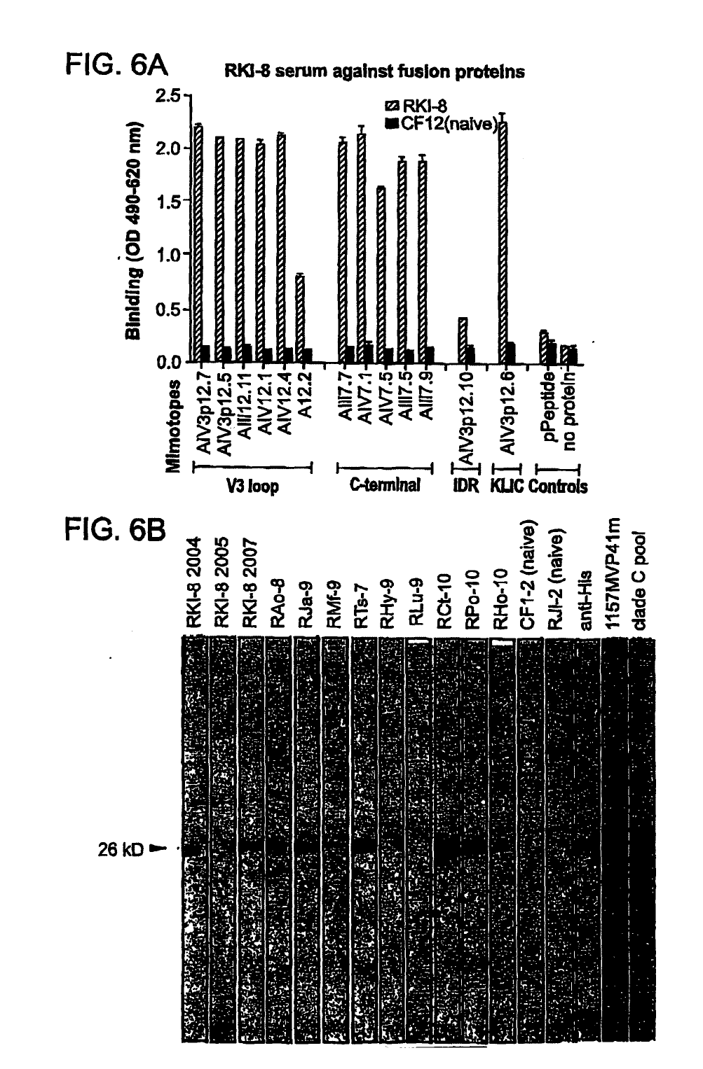

Standard SDS PAGE was performed with 40 μg protein per gel (Bio-Rad). Protein was transferred onto a nitrocellulose membrane (Bio-Rad) using a wet blot apparatus (Bio-Rad). The membrane was cut into strips, blocked with 3% PBSCT and individual strips were incubated with the appropriate serum (1:200) or antibodies (1:1,000 in 3% PBSCT) overnight at 4° C. Membranes were washed 3× and incubated for 1 h at room temperature with an HRP-conjugated antibody (1:2,000 in 3% PBSCT). After washing 5×, the strips were developed with Opti-4CN substrate (Bio-Rad) (FIG. 6).

### Statistical Analysis.

Statistics were calculated using a two-tailed paired t test and only applied to matching pairs of mice comparing the significance between the post-phage boosts and post-gp160 boost (GraphPad Prism 5 for Windows, GraphPad Software).

### Example 2

### Selection of HIV-C Env-Specific Mimotopes

Polyclonal IgG was isolated from a rhesus monkey infected with SHIV-1157ip, an R5SHIV strain encoding env of a recently transmitted Zambian HIV-C. This monkey (animal RK1-8) had developed high-titer, broadly reactive neutralizing antibodies (nAbs) that neutralized primary strains of HIV-1 clades B and C (FIG. 2). Serum IgG was immobilized on paramagnetic beads used to screening three different phage-displayed random peptide libraries (7mer, cyclic 7mer, 12mer). For each screening, 94 single clones were tested in phage ELISA for their specificity using SHIV-positive and SHIV-negative serum in parallel. Positive clones were amplified and sequenced. Peptide insert sequences were grouped according to their motifs and analyzed for linear homology to parental gp160_{SHIV-1157ip}. 78 different clones were obtained; 9 clones represented mimotopes of the V2 loop of gp120, 21 clones represented mimotopes of the V3 loop of gp120 and 8 clones represented mimotopes of the C-terminal domain of gp120. The remainder thirty-four clones resembled regions on gp41 (the majority of the clones represented a subdomain of the immunodominant region (IDR), which is referred to as the immunodominant loop (Zolla-Pazner S (2004). Nat Rev Immunol 4: 199-210) and contains the KLIC motif (20 clones); seven clones shared homology with IDR outside the immunodominant loop as well as several amino acid residues of the N-terminal heptad repeat. Seven other clones showed homology to the MPER. Six phage inserts exhibited no apparent linear similarity to gp160_{SHIV-115ip}.

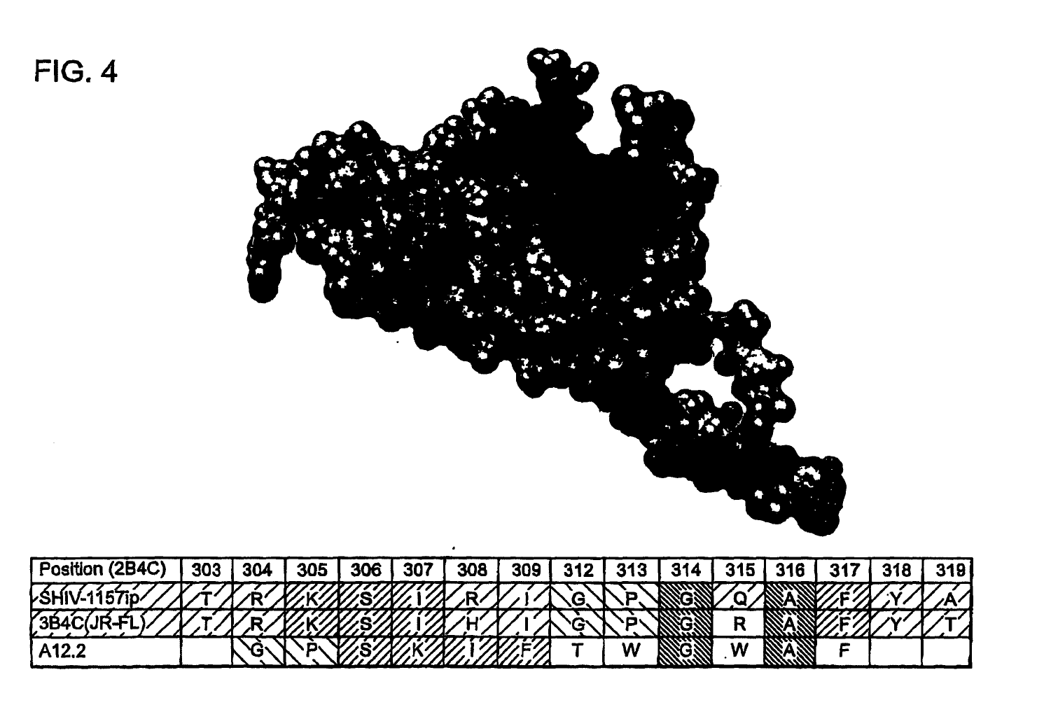

Since several mimotopes displayed only minimal linear homology to gp160_{SHIV-1157ip}, these were believed to exhibit predominantly conformational homology. Using 3DEX software and a published gp120 structure conformational mimotopes were identified. This approach identified a V3 mimotope which combines linear with structural homology (FIG. 2, clone A12.2; FIG. 4). The primary envelope sequence of the structure file (PDB-ID: 2B4C; HIV-1 subtype B strain JR-FL) and SHIV-1157ip was compared. The amino acids in that stretch of V3 are almost identical in sequence (inset FIG. 4). 3DEX allowed identification of two motifs: one that comprises four amino acids at the crown of the V3 loop (FIG. 4, yellow/orange); and a second one that consists of another four amino acids located near the crown (FIG. 4, green). Even though these eight amino acids are discontinuous, they are found in neighboring locations at the Env surface.

### Example 3

### Identification of a Conformational Mimotope of HIV-C Env

To confirm that antibody binding to mimotope A12.2 involves interaction with a conformational epitope and thus depends on the structural integrity of the target HIV-C gp160, antibody binding, under native and reduced conditions to the region on HIV-1 Env represented by the mimotope was assessed. Antibodies from the polyclonal rhesus monkey serum were first isolated by affinity purification with immobilized recombinant phage. The phage-affinity-purified antibodies were then subjected to a dot spot analysis with homologous, trimeric gp160_{SHIV-1157ip} immobilized under native and denaturing conditions (FIG. 5).

Binding of antibodies specific for clone A12.2 was abolished when trimeric gp160 was denatured (FIG. 5, field A1 and B1), although specific binding was demonstrated to native Env. As a control antibodies specific for a phage clone with greater linear homology to the V3 loop (AIV12.4) were used. As expected, these antibodies recognized the native as well as the denatured forms of gp160 (FIG. 5, field A2 and B2). The control without spotted gp160 (FIG. 5, row C) showed no nonspecific antibody binding on any strips. An additional positive control involved spotting anti-monkey IgG (FIG. 5, row D) and confirmed that equal amounts of phage-affinity-purified antibodies had been applied to all strips.

### Example 4

### Reactivity Profile of Mimotopes in the Context of Fusion Proteins

To analyze whether the HIV-1 Env mimotope motif groups represent common antibody epitopes found in sera with broadly reactive nAbs, selected mimotopes of all groups (except MPER) were cloned into fusion proteins and tested by ELISA and Western blot analysis using a panel of rhesus monkey sera with broadly neutralizing activity against different HIV-1 clades. All monkeys showed broadly reactive nAbs against the homologous SHIV-1157ip as well as against a heterologous HIV-1 clade C (pIndieC) and clade B (HIV_{SF162.Ls}). In addition to screening with serum from monkey RK1-8, sera from monkeys RAo-8, RJa-9 and RMf-9 were used for individual phage display selections. The analysis revealed similar motif patterns to RK1-8, including mimotopes representing immunodominant regions such as V3 and KLIC, but also identified new motifs representing stretches of the Kennedy peptide in gp41 and as yet unidentified regions.

To assess if the mimotopes in the context of a fusion protein have conserved their structure, ELISAs was performed with sera from RK1-8. All mimotope fusion proteins were recognized by serum antibodies of RK1-8 (FIG. 6A). Of note, there was also binding to the conformational mimotope A12.2. Individual serum samples from 11 rhesus monkeys that included nine other monkeys with broadly reactive nAbs and two naïve animals displayed cross-reactivity of the KLIC mimotope with most of the sera from the monkey cohort with high-level nAbs. Notably, several rhesus monkey sera recognized selected V3 mimics, especially the two mimotopes AIV3p12.5 and AIV12.4. Motifs isolated with serum from monkey RK1-8 representing the conserved C-terminus of gp120 and the IDR did not react with sera from our rhesus monkey panel, although reactivity was seen with autologous serum. Two naïve control sera did not detect the fusion proteins and a control fusion protein (pPeptide; empty backbone of fusion protein without mimotope insert) was not detected by any of the rhesus monkey sera.

To confirm the ELISA results, all rhesus monkey sera were tested for specific binding to the fusion proteins by Western blots. RK1-8 and other monkey sera from various time points detected the AIV12.4 mimotope. The naïve control sera CF12 and RJi-2 did not bind to the mimotope fusion protein; whereas the positive anti-His antibody control detected the fusion protein migrating at a MW of 26 kD. Further parental serum from the HIV1157i-infected human (1157MVP41m) and a serum pool of HIV-C-infected individuals; both recognized the mimotope (FIG. 6B).

Thus, the ELISA and Western blot results show that the mimotopes expressed in the context of a fusion protein conserved their structure and were recognized by the monkey serum from which they were selected as well as by other sera from our cohort.

### Example 5

### Immunization Studies in Mice

To assess if the mimotopes representing different regions of HIV Env are immunogenic, vaccine studies in mice were performed. Only single priming with a DNA vector encoding the entire gp160, which does not generate binding or nAb responses was used. It was reasoned that a single DNA priming would imprint the immune system with the correct image of native gp160 structures, without diverting antibody responses to immunodominant but ultimately unimportant Env regions. Instead, it was hypothesized that boosting with mimotopes would allow the manipulatation of humoral immune responses by focusing them on important structural domains.

Specifically, first a DNA inoculation was used to prime the immune system with the entire HIV-1 Env. This was followed by phage boosting to focus the antibody response to a certain area of gp160.

Recombinant phages were grouped according to their peptide motifs and were combined into five mixtures to immunize mice (FIG. 2). First, all mice received one priming immunization with a DNA vector encoding gp160_{SHIV-1157ip}; this single DNA inoculation was previously shown to be insufficient for induction of binding antibodies or nAbs. All mice were then boosted four times with phage particles (intervals of 4-5 weeks) and their immune sera were assessed for binding abs and nAbs (FIG. 7).

Antibody binding titers for each group was measured for binding to gp160_{SHIV-1157ip} by ELISA after the four phage boosts. Mean titers ranged from 1:125 to 1:1362, and almost all mice developed anti-Env titers. The lowest titer was observed the MPER group, the highest in the C-terminal domain.

Post-phage boost sera (4^{th} bleed) were tested for neutralization of a heterologous HIV-1 clade B strain, HIV_{SF162.LS}; 59% of the animals had measurable, cross-clade anti-HIV-1 nAbs. The mean IC₅₀ in all groups ranged from 1:19 to 1:70. Surprisingly, four out of the six mice immunized with mimotopes representing the C-terminal domain had nAbs, including two animals with IC₅₀ values >1:100. This is believed to be the first report of the C-terminus of gp120 being linked to the induction of nAb responses.

To examine whether DNA prime/recombinant phage boosts had induced maximal nAb responses or if a boost with native, multimeric gp160 would be beneficial, 11 mice were boosted (6 from the V3, 1 from the C-terminal, 2 from each, the IDR and KLIC group) with gp160_{SHIV-1157ip} and compared with the immune sera from those obtained after phage-boosts only. Mean Env-ELISA titers were 1:1,220 after four phage boosts compared to 1:2,748 after the additional gp160 boost, an increase that did not reach statistical significance. When tested for nAb responses against homologous SHIV-1157ip or heterologous HIV-1_{SF162.LS}, the additional Env boost did not significantly raise nAb titers against either virus (p=0.8544 and p=0.3935, respectively; Data not shown, Humbert et al. PLOS One Manuscript (Submitted)), implying that maximal responses had been induced by DNA prime/phage boosting. Thus, the higher ELISA antibody titer after protein boosting did not translate into better nAb titers.

### Example 6

### Analysis of Vaccination-Induced Antibodies

The ability of antibodies induced by the different immunogens to react with native versus denatured HIV-1 gp160 was determined. It is possible that the mixture of similar but not identical mimotopes is able to broaden the immune response and to induce antibodies against conformational epitopes rather than linear ones. The V3 mimotopes show incomplete linear homology to gp160 and contains structure-specific homologies. In contrast, the gp120 C-terminal mimotopes were primarily linear.

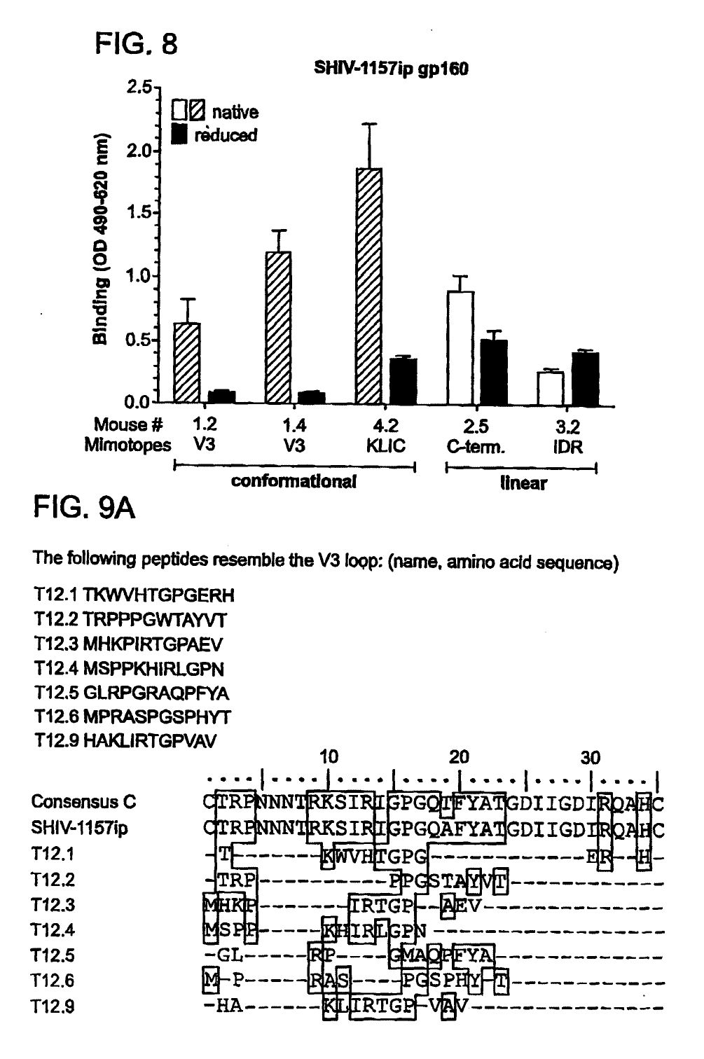

The post-protein boost sera were tested (5^{th} bleed) for reactivity against native and reduced HIV-1 gp160 by ELISA (FIG. 8). Sera from two mice boosted with V3-loop mimotopes showed decreased binding upon denaturation of Env (7-14-fold). In contrast, the signal obtained with serum from the mouse boosted with mimotopes representing the HIV-1 gp120 C-terminus decreased only 1.7-fold. These data imply that the V3-loop mimotopes induced conformational antibody responses, whereas the gp120 C-terminal mimotopes induced predominantly linear antibody responses.

Mimotope-induced antibodies against the gp41 IDR (FIG. 8; mouse #3.2) showed slightly increased binding upon denaturation of gp160_{SHIV-1157ip}; whereas serum antibodies from mouse #4.2 immunized with the potentially conformational KLIC motifs exhibited decreased binding after target denaturation (FIG. 8), implying that they are specific for conformational epitopes.

### Example 7

### Hybridoma Production

A mouse immunized with V3 loop mimotopes (SEQ ID NOs: 21, 23, 25, 28, 29 and 31) was used to generate monoclonal antibodies (mAbs). 14 positive wells were identified and four were picked for a subcloning procedure. All four clones were shown to be monoclonal and producing IgG1 (19-40 μg/ml medium). These four clones were designated Group 1 in FIG. 2.

IgG1-positive supernatants from all four monoclonal hybridomas were tested for their ability to bind to HIV Env preparations made from different strains. Specific binding was observed not only to Env from the homologous virus, but also to Env from HIV₁₀₈₄ᵢ (clade HIV_{Bal} (clade B), and HIV_{SF162} (clade B). Therefore, the IgG1 secreted by the monoclonal mouse hybridomas recognize HIV Env domains that are conserved across different clades.

Supernatants with defined antibody concentrations were tested for their ability to neutralize HIV_{SF162.Ls}. Two out of the three supernatants were able to neutralize the virus (IC₅₀ levels: ˜10 μg/ml).

Also, mAb generation from one mouse immunized with C-terminal mimotopes was initiated. 13 positive were identified and four subcloned. All clones bind various Env proteins from different strains (as described above). Neutralization assays against the clade B virus SF162.LS showed neutralizing activity for these clones (not knowing the secreted antibody concentration in the medium).

As reported above, these results demonstrate the isolation of mimotopes specific for HIV-C Env, selected with serum from a clade C SHIV-infected rhesus monkey with broadly reactive nAbs against different clades of HIV. Using the 3D-structure of the clade B HIV_{JR-FL} in conjunction with 3DEX software analysis, Env mimics were identified that reflect structurally conserved elements and/or conserved amino acid stretches, including motifs representing V3 and the C-terminal domain of gp120, as well as the IDR, the KLIC motif, and MPER of gp41. 3DEX identified a conformational V3 mimotope with only minimal linear sequence homology. The conformational characteristics were confirmed experimentally by differential recognition of native and denatured Env using phage-affinity purified antibodies specific for this V3 loop mimotope. DNA priming with a HIV-C gp160-encoding vector followed by repeated boosting with mimotope-encoding phages induced antibodies in mice that specifically bound to HIV gp160 and neutralized viruses with clade B and C envelopes.

The key for a successful mimotope lies in the possibility of its identification. Earlier attempts at identifying Env mimotopes only had one tool available: sequencing of the peptide insert. An important advance that finally allows identification based upon 3D structure was the development of the 3DEX and similar software. This new approach allowed identification of the mimotope A12.2 and have demonstrated differential recognition of native versus denatured HIV-C gp160 by phage affinity-purified rhesus monkey abs. These data demonstrate that the use of random peptide phage display to select disease-specific mimotopes from a large library in combination with computational analysis will likely lead to the identification of novel immunogens.

For one V3 mimotope (A12.2), gp160 conformation was shown to be important for binding of its cognate phage affinity-purified antibody. The V3 loop is thought to adopt a highly specific and conserved structure to mediate coreceptor interaction. Thus, even if variable in its sequence, the specificity of the loop in its selective interaction is likely to be a target for antibody-mediated neutralization. The identified mimotopes likely represent conformational mimics of this loop and were able to induce nAbs in mice.

Since biopanning using sera from other monkeys that also exhibited broad neutralizing activity resulted in the isolation of phage peptides similar to those selected with serum from animal RK1-8, their cross-recognition pattern was tested by ELISA using mimotope fusion proteins. Surprisingly, no cross-recognition of the conserved C-terminal domain mimotopes was observed, but several mimotopes of the V3 loop showed specific binding to other monkey sera. Cross-recognition of epitopes or mimotopes by various sera from HIV-infected individuals showing high nAb-titers is frequently equated with its potential ability to induce effective antibodies. Phage display with HIV-positive sera primarily results in the identification of immunodominant regions, such as KLIC, because of the abundance of antibodies targeting this region. However, the frequency with which an epitope was recognized is not linked to neutralization and mimotopes with no or limited cross-reactivity may be of interest to nAb generation. The rhesus monkeys in the cohort may have developed an unusually effective immune response against Env in the context of natural infection, perhaps due to the fact that the SHIV-C, SHIV-1157ip, encodes the env gene of a recently transmitted virus. Interestingly, newly infected partners from a cohort of HIV discordant couples not only harbored HIV-C strains that were more easily neutralized than contemporaneous virus from the infected source person, but the recently transmitted HIV-C isolated also had shorter V-loops. It is possible that Env molecules of recently transmitted HIV-C have a more open configuration that favors the induction of broadly reactive nAbs. The broad neutralization reactivity of these rhesus monkey sera could be useful in identifying non-immunodominant but structurally conserved domains. Even though in the context of a natural infection this may be a rare response and not occur in each host, it could potentially be induced with epitope mimics which focus the immune response only to the part linked to nAbs.

Previous studies have shown that whole phage particles can be used to induce peptide-specific antibodies and protective immune responses in animals. The immunization described above with mimotope mixtures differs in a significant way from these earlier studies, which showed limited efficacy. Instead of using a series of multiple immunizations with recombinant phages expressing mimotopes as used by this group, a novel DNA prime/recombinant phage boost strategy was employed. A single priming with a DNA vector encoding the entire gp160 was used, which will not generate binding or nAb responses; it was reasoned that a single DNA priming would imprint the immune system with the correct image of native gp160 structures, without diverting ab responses to immunodominant but ultimately unimportant Env regions. Instead, it was hypothesized that the boosting with mimotopes would allow manipulation of humoral immune responses by focusing them on important structural domains. This promising immunofocusing approach was shown to be effective in eliciting anti-Env abs. The immunization regimen consists of immunogens which focus the immune response on one or a few neutralizing epitopes. This potentially results in a larger proportion and higher titer of nAbs. The boosts were performed with five different mimotope groups, each representing a well-defined Env region (the V3 loop, the C-terminus of gp120, gp41 IDR, and separately, the KLIC motif, and MPER). This approach provided for the identification of novel HIV Env regions capable of inducing nAbs in the absence of the otherwise overpowering influence of immunodominant regions. For the first time, the C-terminal domain of gp120, previously identified as immunogenic but never linked to nAb responses, was shown to induce nAbs. Interestingly, the DNA prime/phage mimotope boosts strategy appeared to have induced the maximal nAb responsiveness to a given domain, since additional boosting with the native, multimeric gp160 failed to significantly raise the nAb titers.

These results show that the combination of phage display and 3DEX analysis is a powerful tool to analyze the humoral immune response by identifying novel antigenic mimics that represent functionally conserved Env domains, such as V3 or the C-terminal domain of gp120. Using an innovative DNA prime/phage boost regimen, cross-clade nAbs were induced against HIV clades B and C and for the first time, linked the C-terminal domain of gp120 to antibody-mediated neutralization in mice.

### Example 8

### Positive/Negative Screening Identifies V3 and C-Terminal Domain Mimotopes

Phage display was used to screen a commercially available random peptide libarary for binding to antibodies from RK1-8, a rhesus monkey that had high titers of cross-clade neutralizing antibodies. To specifically select for mimic recognizing conformational antibody epitopes, RK1-8 serum was pre-incubated with native and in a separate reaction with denatured SHIV-1157ip gp160. Bound antibodies were eluted and used for a biopanning procedure with positive selections (using the antibodies eluted from the native gp160) and negative counter-selections (using the antibodies eluted from the denatured gp160.

Interestingly, only two very distinctive groups of peptides resembling two different regions of gp120, the V3 loop and the C-terminal domain, were selected. Even though the V3 loop is very variable in sequence, antibodies targeting the conserved structure of this loop induce neutralization. This is the first identification of such antibodies, which are of particular interest because they are expected to recognize conformational epitopes on the HIV envelope structure. Significantly, the envelope mimics described herein (SEQ ID NOs: 94-109) are likely to induce antibodies that target conserved conformational epitopes on the HIV envelope glycoprotein and lead to potent neutralization by mimicking the complex three-dimensional structure rather than a linear amino acid sequence.

### Example 9

### Epitope-Specific B Cell Isolation with Mimotopes

Mimotopes of the invention are also useful for the isolation of epitope-specific B cells. These methods provide for high sensitivity and high yield of epitope-specific B cells without the need for library construction or panning. In one embodiment, a mimotope of the invention (e.g., SEQ ID NOs: 1-84 and 94-109) is labeled with a detectable marker (e.g., fluoroscein). In other embodiments, the mimotope is expressed as a fusion protein with a detectable marker (e.g., a fluorescent protein, such as GFP, YFP, BFP, CFP). B cells are isolated from bone marrow, lymph nodes, spleen, or peripheral blood of a seropositive or HIV-positive subject. If desired, the samples are enriched for B cells using any method known in the art. In one approach, the number of B cells in a sample is increased using a commercially available kit, such as the Human B Cell Enrichment Kit (StemSep®), which is available from Stem Cell Technologies. The sample is stimulated to induce the production of surface receptor molecules that are specific for a protein target represented by the mimotopes (e.g., the HIV envelope). In one approach, a sample comprising or enriched for B cells is contacted with a detectably labeled mimotope (e.g., SEQ ID NOs: 1-84 and 94-109). Detectably labeled B cells are then selected using flow cytometric sorting to isolate epitope-specific B cells. Messenger RNA is isolated from the cells, reverse transcribed, and the immunoglobulin genes from the cells are subsequently isolated. A gene encoding a mimotope-specific monoclonal antibody may be inserted into a cell line suitable for monoclonal antibody production. Accordingly, this method provides for the isolation of antibodies specific for the mimotope used in the selection. Such methods are advantageous because they obviate the need for generation of hybridomas for monoclonal antibody production.

### Example 10

### Isolation of Epitope-Specific B Cells Using HIV Mimotopes

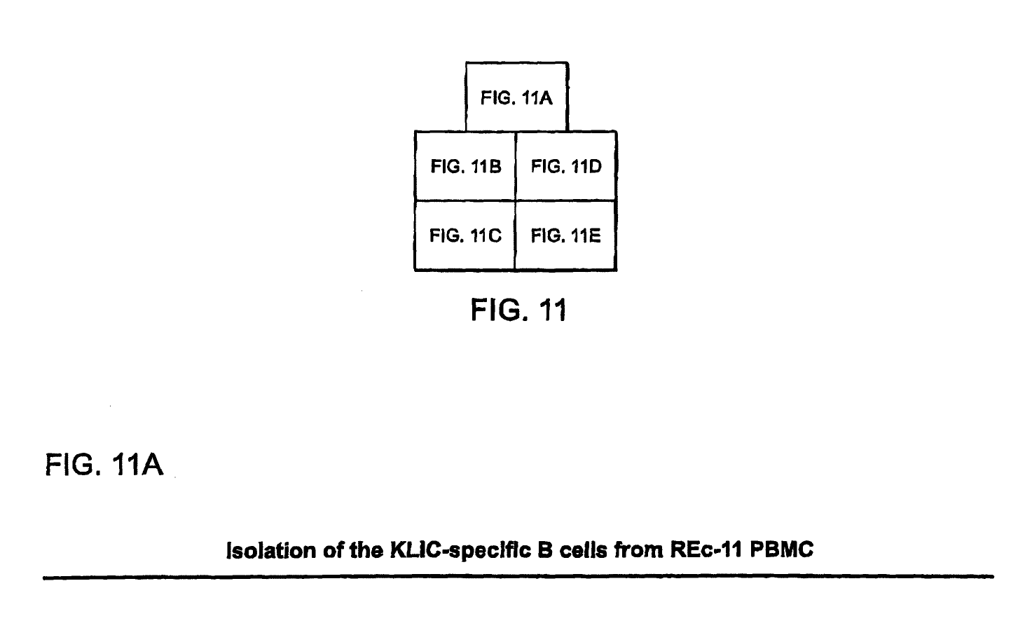

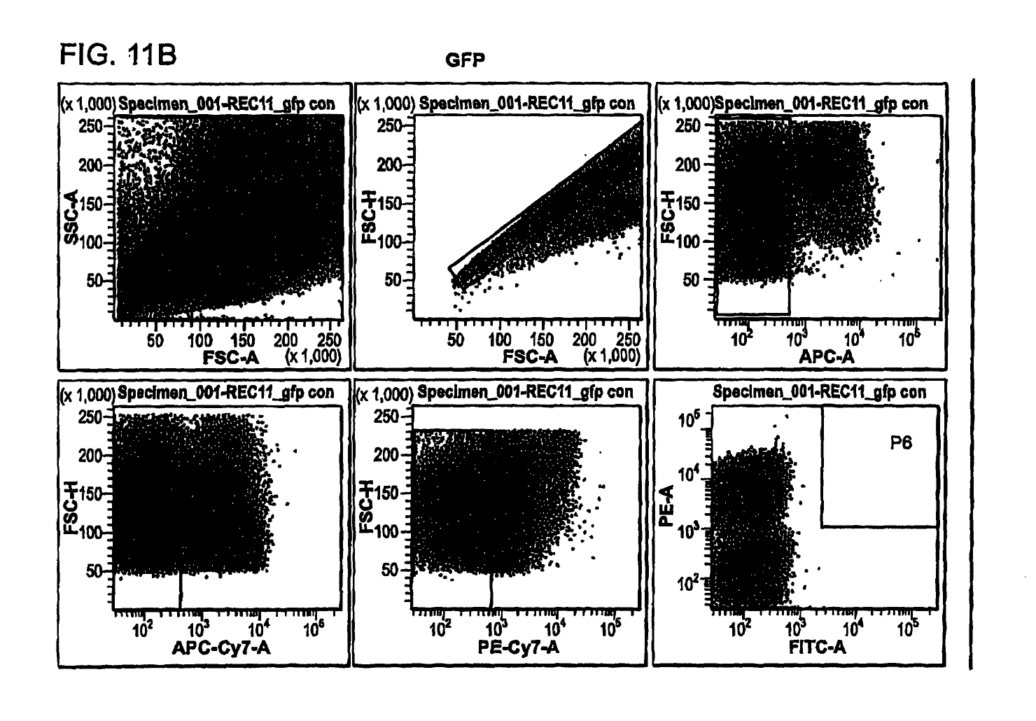

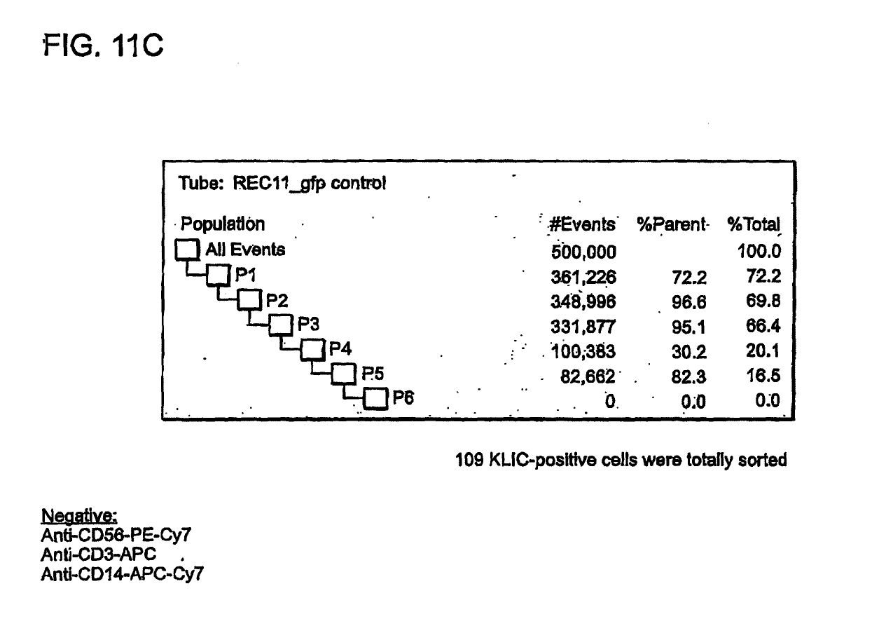

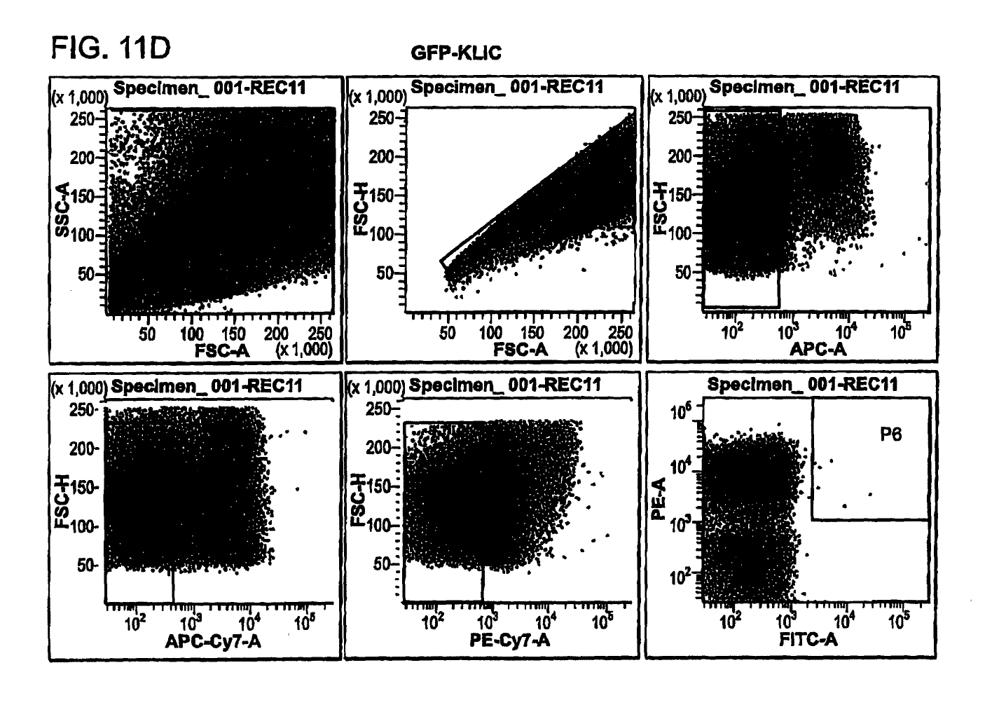

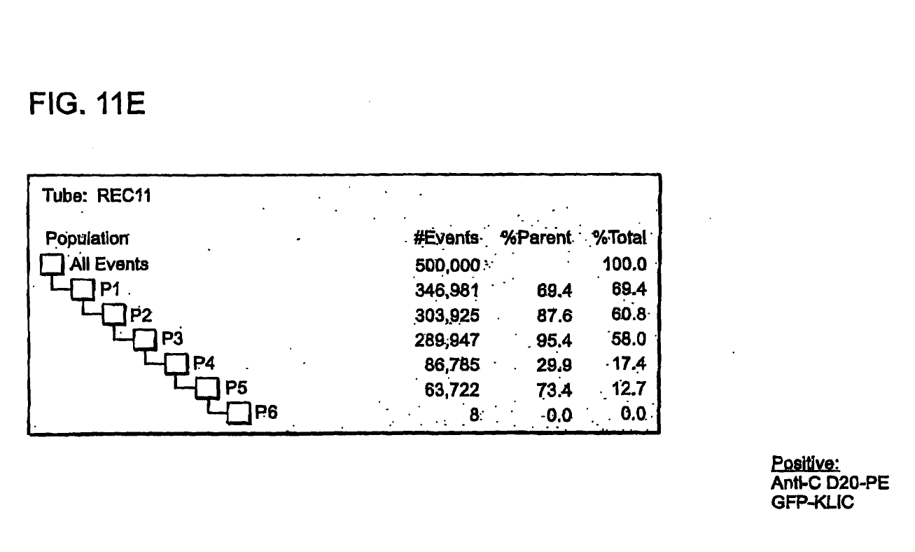

Rhesus monkeys selected for this study were vaccinated using different schemes and challenged with pathogenic R5SHIVs. For detection of mimotope-specific B cells expressing cell surface Ig, peripheral blood mononuclear cells (PBMC) from rhesus monkeys were incubated with a mixture of anti-CD56 labeled with phycoerythrin (PE)-Cy7, anti-CD3-APC, anti-CD14-APC-Cy7, anti-CD20-PE, or anti-CD19-PE as a marker for B cells (all commercially available from “BD Pharmingen”) and GFP-mimotope fusion proteins. The following mimotopes were used for a GFP-mimotope fusion protein generation: gp120 C-terminal—AIII 7.5 (SEQ ID NO: 38), AIII 7.7 (SEQ ID NO: 34), AIII 7.9 (SEQ ID NO: 39), AIV7.1 (SEQ ID NO: 35), AIV7.5 (SEQ ID NO: 37); gp120 V3-loop A12.2 (SEQ ID NO: 31) and gp41 KLIC-mimotope AIV3p12.6 (SEQ ID NO: 66). GFP alone was used as control. For detection of GFP fluorescence the FITC filter was used. After gating lymphocyte fraction and excluding cell doublets, CD56⁻, CD14⁻, CD3⁻, CD20⁺, or CD19⁺, GFP cells were analyzed and sorted (FIG. 11). The panel at the left of FIG. 11 shows cell sorting for a GFP-only control. The panel at the right of FIG. 11 shows cell sorting for cells contacted with a GFP-KLIC fusion protein (SEQ ID NO: 66) GFP-positive cells were seen in control staining whereas GFP-KLIC allowed the isolation of 109 mimotope-specific B cells. FIG. 11 shows the gating strategy. Table 1 gives a summary of different sorting experiments demonstrating the isolation of mimotope-positive B cell using different mimotopes.

TABLE 1
B cell sorting summary table.

| | | | | Number of | B cell |
|---|---|---|---|---|---|
| # | Monkey | Mimotope | Source | B cell isolated | marker |
| 1 | RHo-10 | AIV3p12.6 | PBMC | 48 | CD20 |
| 2 | RJa-9 | | PBMC | 291 | CD20 |
| 3 | REc-11 | | PBMC | 109 | CD20 |
| 4 | RJr-11 | | PBMC | 64 | CD20 |
| 5 | RVe-10 | AIII7.9 | PBMC | 49 | CD20 |
| 6 | RJr-11 | AIV3p12.6 | PBMC | 1407 | CD20 |
| 7 | RHo-10 | | PBMC | 259 | CD20 |
| 8 | RJa-9 | | PBMC | 1240 | CD20 |
| 9 | RVe-10 | AIII7.9 | PBMC | 77 | CD20 |
| 10 | RWc-10 | | PBMC | 59 | CD20 |
| 11 | RJa-9 | AIV3p12.6 | BM | 295 | CD20 |
| 12 | RWc-10 | AIII7.9 | BM | — | CD20 |
| | | | PBMC | 37 | |
| 13 | RVe-10 | AIII7.9 | BM | 6 | CD20 |
| 14 | RHb-10 | A12.2 | BM | 32 | CD20 |
| | | | PBMC | 96 | |
| 15 | RWc-10 | Mix of AIII7.5, AIII7.7, | PBMC | 107 | CD19 |
| | | AIII7.9, AIV7.1, AIV7.5 | | | |
| 16 | RVe-10 | Mix of AIII7.5, AIII7.7, | PBMC | 132 | CD19 |
| | | AIII7.9, AIV7.1, AIV7.5 | | | |
| 17 | RHb-10 | A12.2 | PBMC | 1039 | CD19 |
| 18 | RJr-11 | AIV3p12.6 | PBMC | 1501 | CD19 |
| 19 | RWc-10 | Mix of AIII7.5, AIII7.7, | PBMC | 281 | CD19 |
| | | AIII7.9, AIV7.1, AIV7.5 | | | |
| 20 | RVe-10 | Mix of AIII7.5, AIII7.7, | PBMC | 114 | CD19 |
| | | AIII7.9, AIV7.1, AIV7.5 | | | |
| 21 | RHb-10 | A12.2 | PBMC | 2019 | CD19 |
| 22 | RJr-11 | AIV3p12.6 | PBMC | 160 | CD19 |

The term “PBMC” denotes peripheral blood mononuclear cells.
“BM” denotes bone marrow cells.

Table 1 summarizes results obtained using HIV mimotopes to isolate mimotope-specific B cells from rhesus monkey PBMC or bone marrow (BM) cells. The mimotopes used represent different parts of HIV gp41 and gp120 envelope proteins, including gp120 C-terminal—AIII 7.5 (SEQ ID NO: 38), AIII 7.7 (SEQ ID NO: 34), AIII 7.9 (SEQ ID NO: 39), AIV7.1 (SEQ ID NO: 35), AIV7.5 (SEQ ID NO: 37); gp120 V3-loop A12.2 (SEQ ID NO: 31) and gp41 KLIC-mimotope AIV3p12.6 (SEQ ID NO: 66).

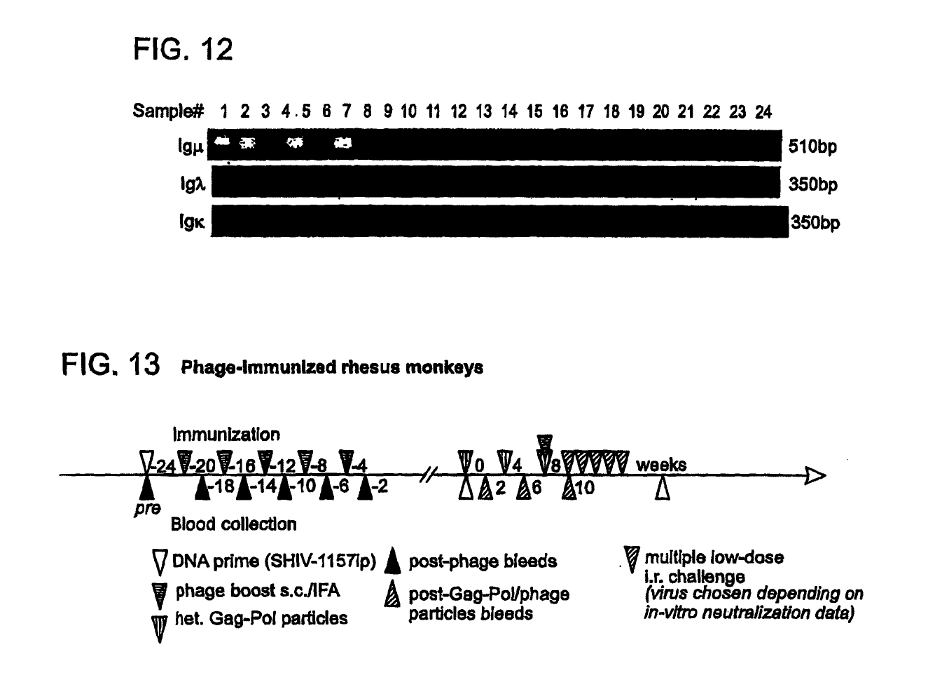

Single-sorted mimotope-specific B cells isolated from macaque RVe-10 (sortings #9 and #20 Table 1) were subjected to RT-PCR. FIG. 12 depicts gel images showing RT-PCR products of Igκ, Igμ and Igλ DNA fragments were further purified and sequenced. Sequence analysis with the immunoglobulin sequence database ImmunoGenetics Database (imgt.cines.fr) showed conformity of PCR fragments to the corresponding Ig variable genes types (Table 2). All B cells were monoclonal, because only one heavy or light chain was amplified by RT-PCR for each single-sorted B-cell. All matching V gene fragments derived from the same B cell are used to generate recombinant antibodies.

TABLE 2
Matching pairs of light and heavy chain V genes from single-sorted
mimotope-specific rhesus monkey B cells.

| Sorting # | Monkey | B cell position | Ig chain | V gene |
|---|---|---|---|---|
| 9 | RVe-10 | A4 | lambda | IGLV1-47 |
| | | | gamma | IGHV5-51 |
| 20 | RVe-10 | A1 | kappa | IGHV1-12 |
| | | | mu | IGHV4-4 |
| | | A2 | lambda | IGLV1-40 |
| | | | gamma | IGHV3-15 |
| | | B3 | lambda | IGLV2-11 |
| | | | mu | IGHV4-4 |
| | | B11 | lambda | — |
| | | | mu | IGHV3-15 |
| | | D1 | kappa | IGKV2-30 |
| | | | mu | IGHV3-71 |
| | | F5 | kappa | IGKV1-17 |
| | | | mu | IGHV4-4 |
| | | F8 | kappa | IGKV1-12 |
| | | | mu | — |
| | | G1 | kappa | IGKV1-9 or IGKV1-13 or |
| | | | | IGKV1-16 |
| | | | mu | IGHV3-15 |
| | | H1 | kappa | IGKV1-17 |
| | | | mu | IGHV3-11 or IGHV3-48 |
| | | H3 | lambda | IGLV2-11 |
| | | | mu | IGHV3-74 |

### Example 11

### Immunization Studies in Rhesus Monkey Studies

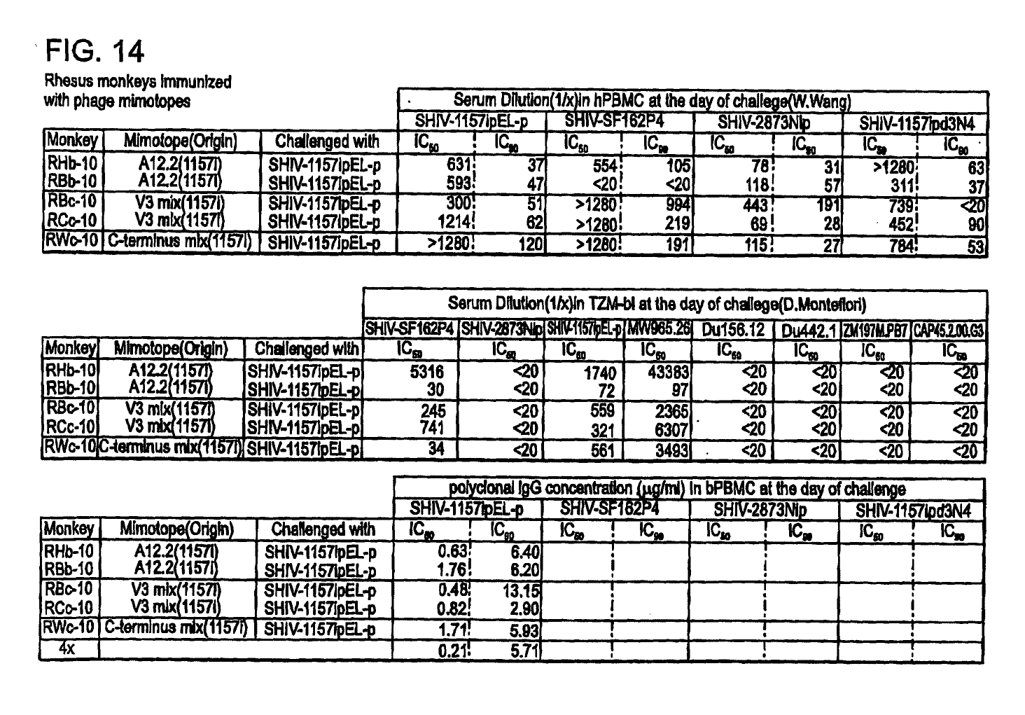

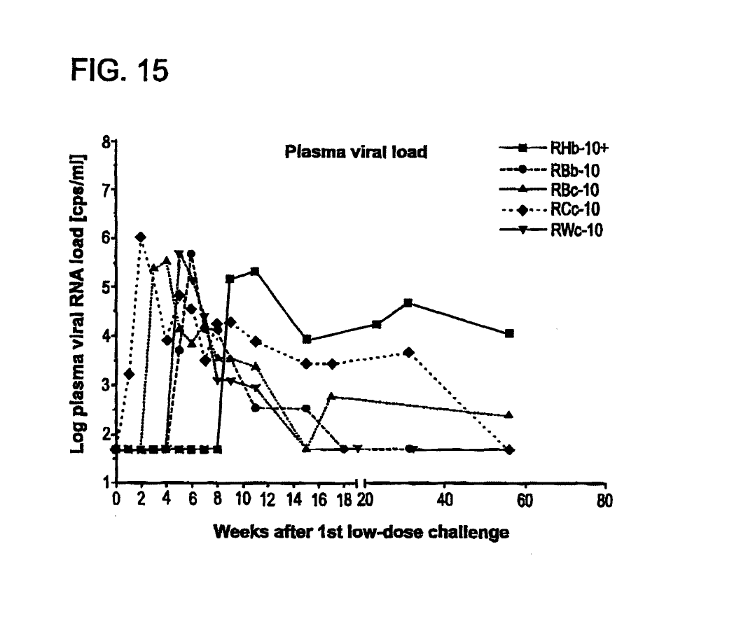

Mimotopes of the HIV clade C envelope were identified that represented subdomains of conserved areas on the HIV envelope protein, including mimics that resembled the V3 loop and the C-terminal domain of gp120. The mimotopes used in immunization studies in mice to induce neutralizing antibodies (nAbs) against HIV (Example 5), were used in rhesus monkey studies. The nAb immune response to these subdomains was foused in a novel prime/boost strategy in combination with immunogens triggering the cellular arm of the immune system (FIG. 13). Rhesus monkeys were primed once with DNA of the entire HIV-C envelope and subsequently boosted 5× with phage particles. The rhesus monkeys were boosted with heterologous Gag-Pol particles twice and a third time in combination with another phage particle boost. The rhesus monkeys were challenged with a heterologous clade C virus in a 5 times low, one times high-dose regimen. Cross-clade nAbs were induced in all six rhesus monkeys (FIG. 14). The vaccination showed partial protection during a multiple low-dose, heterologous SHIV challenge resulting also in a lower peak viremia during acute infection (FIG. 15).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of inducing neutralizing antibodies in a subject, the method comprising
(a) administering to the subject an expression vector encoding an antigen in an amount insufficient to induce an antibody response in the subject, and
(b) administering to the subject an effective amount of a polypeptide comprising a mimotope of the antigen, wherein the method induces a neutralizing antibody response in the subject.

2. The method of claim 1, wherein the antigen is derived from a pathogen.

3. The method of claim 2, wherein the pathogen is a bacteria, virus, fungus, protozoan, or parasite.

4. The method of claim 1, wherein the expression vector is a DNA vector.

5. A method for isolating an epitope-specific or mimotope-specific B cell, the method comprising
contacting an antibody expressing B cell with a detectable fusion polypeptide comprising a mimotope of a specific antigen of interest, thereby labeling the B cell; and
isolating a labeled epitope-specific or mimotope-specific B cell.

6. The method of claim 5, wherein the detectable fusion polypeptide comprises a reporter selected from the group consisting of GFP, YFP, BFP, and CFP.

7. The method of claim 5, wherein the reporter is GFP.

8-11. (canceled)

12. A method of preventing or treating a disease in a subject comprising:
administering to the subject an effective amount of a polypeptide comprising a mimotope of an antigen to induce or enhance an immune response in the subject, thereby preventing or treating the disease in the subject.

13. The method of claim 12, wherein the disease is a bacterial infection, a viral infection, a fungal infection, a protozoan infection, a parasitic infection, or autoimmune disease.

14. An isolated HIV envelope-like polypeptide, wherein said polypeptide comprises an amino acid sequence which is 90% or more identical to an amino acid sequence selected from the group consisting of those recited in FIG. 1, FIG. 16, or provided at SEQ ID NOs 94-109.

15. The isolated HIV envelope-like polypeptide of claim 14, wherein said polypeptide is a mimotope having an amino acid sequence which is 90% or more identical to an amino acid sequence selected from the group consisting of SEQ ID NOs: 1-84 or 94-109.

16-20. (canceled)

21. The isolated HIV envelope-like polypeptide of claim 14, wherein said polypeptide is a fusion polypeptide.

22. A composition comprising the HIV envelope-like polypeptide of claim 14 and a pharmacologically acceptable carrier.

23. An immunogenic composition comprising an effective amount of a polypeptide comprising an amino acid sequence having at least about 95% identity to SEQ ID NO: 1-109 or an immunogenic fragment thereof in a pharmaceutically acceptable carrier, or
An immunogenic composition comprising an effective amount of a polypeptide that consists essentially of an amino acid sequence of SEQ ID NO:1-109 or an immunogenic fragment thereof, or
A kit comprising an immunogenic composition comprising a polypeptide comprising or consisting essentially of the amino acid sequence provided at SEQ ID NO: 1-109 or an immunogenic fragment thereof and instructions for use in an immunization regimen.

24-28. (canceled)

29. An isolated nucleic acid encoding one or more of the HIV envelope-like polypeptides of claim 14.

30. A vector comprising the nucleic acid molecule of claim 29.

31. A host cell comprising the vector of claim 30.

32. A composition comprising one or more of the isolated nucleic acids of claim 29 and a pharmacologically acceptable carrier.

33. A kit comprising the composition of claim 32, and instructions for use.

34. A neutralizing antibody that specifically binds to an HIV C-terminal region of gp120, or
An antibody that specifically binds to an HIV envelope-like polypeptide, wherein said polypeptide comprises an amino acid sequence which is 90% or more identical to an amino acid sequence selected from the group consisting of those recited in FIG. 1, FIG. 16, or provided at SEQ ID NOs 1-109.

35-39. (canceled)

50. A composition comprising an effective amount of the antibody of claim 34 and a pharmacologically acceptable carrier.

51-52. (canceled)

53. A kit comprising the composition of claim 50 and instructions for use in treating or preventing an HIV infection or AIDS.

54. A cell line expressing the antibody of claim 35.

55. A method for inducing an immune response in a subject comprising administering a pharmaceutical composition comprising an immunologically effective dose of an HIV envelope-like polypeptide, wherein said polypeptide comprises an amino acid sequence which is 90% or more identical to an amino acid sequence selected from the group consisting of those listed in FIG. 1, FIG. 16, or SEQ ID NOs 1-109.

56. A method of inducing an immune response against an HIV Env epitope in a subject having or at risk of developing an HIV infection or AIDS, the method comprising administering to the subject an effective amount of a polypeptide comprising a mimotope having structural similarity to an Env region selected from the group consisting of the V3 loop, the C-terminus of gp120, gp41 IDR, the KLIC motif, and MPER, thereby inducing an immune response in a subject.

57-59. (canceled)

60. A method of inducing neutralizing antibodies in a subject having or at risk of developing an HIV infection or AIDS, the method comprising
(a) administering to the subject a DNA vector encoding gp160 in an amount insufficient to induce an antibody response, and
(b) administering to the subject an effective amount of a polypeptide comprising an Env mimotope, wherein the method induces a neutralizing antibody response in the subject.

61-69. (canceled)

70. A method for treating or preventing an HIV infection in a subject, the method comprising administering a therapeutically effective dose of an antibody that specifically binds to an HIV envelope-like polypeptide comprising an amino acid sequence which is 90% or more identical to an amino acid sequence selected from the group consisting of those recited in FIG. 1 or FIG. 16.

71-77. (canceled)

78. A detectable polypeptide comprising an amino acid sequence having at least about 90% identity to SEQ ID NOs: 1-109 or an immunogenic fragment thereof in association with a detectable moiety, or
A detectable fusion polypeptide comprising an amino acid sequence having at least about 90% identity to SEQ ID NOs: 1-109 or an immunogenic fragment thereof and a detectable reporter, or
A detectable fusion peptide comprising or consisting essentially of a KLIC mimotope selected from the group consisting of SHGKLLA, TGKLQCI, QHSWSCSGKLLC, SLWTLHGSLISA, and SIWQTSGVLISY, or an immunogenic fragment thereof and a detectable reporter.

79-85. (canceled)

86. A polynucleotide encoding the detectable fusion polypeptide of claim 78.

87. A method for isolating an epitope-specific B cell, the method comprising:
contacting a B cell with a detectable fusion polypeptide of claim 78, or an immunogenic fragment thereof, thereby labeling the B cells; and
isolating an epitope-specific B cell, or
A method for isolating a B cell that specifically binds a KLIC motif of HIV, the method comprising contacting a B cell with a detectable polypeptide comprising a KLIC mimotope or immunogenic fragment thereof, thereby labeling the B cell, and isolating a B cell that specifically binds a KLIC motif of HIV.

88-94. (canceled)

95. A method for monoclonal antibody production, the method comprising
contacting a B cell with a detectable polypeptide comprising a detectable polypeptide of claim 77, or an immunogenic fragment thereof, thereby detectably labeling the B cells;
selecting an epitope-specific B cell,
identifying a gene encoding an epitope-specific immunoglobulin, and
expressing the gene in a cell suitable for monoclonal antibody production.

96-97. (canceled)

98. A monoclonal antibody produced according to the method of claim 95.

99. (canceled)


SEQUENCE LISTING (510 sequences)

SEQ ID NO: 1 - protein, 7 aa, Artificial Sequence
  Description of Artificial Sequence Synthetic peptide
TQIRDKT

SEQ ID NO: 2 - protein, 7 aa, Artificial Sequence
  Description of Artificial Sequence Synthetic peptide
TLIRDKQ

SEQ ID NO: 3 - protein, 7 aa, Artificial Sequence
  Description of Artificial Sequence Synthetic peptide
TQVRDKQ

SEQ ID NO: 4 - protein, 7 aa, Artificial Sequence
  Description of Artificial Sequence Synthetic peptide
TLVRDKQ

SEQ ID NO: 5 - protein, 7 aa, Artificial Sequence
  Description of Artificial Sequence Synthetic peptide
TAIRDKS

SEQ ID NO: 6 - protein, 7 aa, Artificial Sequence
  Description of Artificial Sequence Synthetic peptide
TAIRDKQ

SEQ ID NO: 7 - protein, 7 aa, Artificial Sequence
  Description of Artificial Sequence Synthetic peptide
TMIRDKM

SEQ ID NO: 8 - protein, 7 aa, Artificial Sequence
  Description of Artificial Sequence Synthetic peptide
THREQPS

SEQ ID NO: 9 - protein, 7 aa, Artificial Sequence
  Description of Artificial Sequence Synthetic peptide
TSVKHRL

SEQ ID NO: 10 - protein, 12 aa, Artificial Sequence
  Description of Artificial Sequence Synthetic peptide
IMPLPKPIRTGP

SEQ ID NO: 11 - protein, 12 aa, Artificial Sequence
  Description of Artificial Sequence Synthetic peptide
YIQFPKPIRLGP

SEQ ID NO: 12 - protein, 12 aa, Artificial Sequence
  Description of Artificial Sequence Synthetic peptide
TTPQKVLRLGPF

SEQ ID NO: 13 - protein, 12 aa, Artificial Sequence
  Description of Artificial Sequence Synthetic peptide
ASHWTTKGRLVA

SEQ ID NO: 14 - protein, 7 aa, Artificial Sequence
  Description of Artificial Sequence Synthetic peptide
DTRRMGP

SEQ ID NO: 15 - protein, 7 aa, Artificial Sequence
  Description of Artificial Sequence Synthetic peptide
KEIRLGP

SEQ ID NO: 16 - protein, 7 aa, Artificial Sequence
  Description of Artificial Sequence Synthetic peptide
KYIHLGP

SEQ ID NO: 17 - protein, 7 aa, Artificial Sequence
  Description of Artificial Sequence Synthetic peptide
VRTGPGV

SEQ ID NO: 18 - protein, 12 aa, Artificial Sequence
  Description of Artificial Sequence Synthetic peptide
TIKTIRTEXRNX

SEQ ID NO: 19 - protein, 7 aa, Artificial Sequence
  Description of Artificial Sequence Synthetic peptide
ATWRIGP

SEQ ID NO: 20 - protein, 12 aa, Artificial Sequence
  Description of Artificial Sequence Synthetic peptide
HRPGPGAHHYLS

SEQ ID NO: 21 - protein, 12 aa, Artificial Sequence
  Description of Artificial Sequence Synthetic peptide
IRPHPGHMYYSW

SEQ ID NO: 22 - protein, 12 aa, Artificial Sequence
  Description of Artificial Sequence Synthetic peptide
AARFGPGMPFYH

SEQ ID NO: 23 - protein, 12 aa, Artificial Sequence
  Description of Artificial Sequence Synthetic peptide
QVRMGPGQPDYL

SEQ ID NO: 24 - protein, 12 aa, Artificial Sequence
  Description of Artificial Sequence Synthetic peptide
VRPPPGQPHFTF

SEQ ID NO: 25 - protein, 12 aa, Artificial Sequence
  Description of Artificial Sequence Synthetic peptide
VRLPPGASGYTP

SEQ ID NO: 26 - protein, 12 aa, Artificial Sequence
  Description of Artificial Sequence Synthetic peptide
VRLPPGASGYTP

SEQ ID NO: 27 - protein, 12 aa, Artificial Sequence
  Description of Artificial Sequence Synthetic peptide
ALXPGPGSPLFT

SEQ ID NO: 28 - protein, 12 aa, Artificial Sequence
  Description of Artificial Sequence Synthetic peptide
IRPGPAAGGYPA

SEQ ID NO: 29 - protein, 12 aa, Artificial Sequence
  Description of Artificial Sequence Synthetic peptide
KMIHLGPQQTFP

SEQ ID NO: 30 - protein, 12 aa, Artificial Sequence
  Description of Artificial Sequence Synthetic peptide
KMIHLGPQQTFP

SEQ ID NO: 31 - protein, 12 aa, Artificial Sequence
  Description of Artificial Sequence Synthetic peptide
GPSKIFTWGWAF

SEQ ID NO: 32 - protein, 7 aa, Artificial Sequence
  Description of Artificial Sequence Synthetic peptide
STFTWSW

SEQ ID NO: 33 - protein, 7 aa, Artificial Sequence
  Description of Artificial Sequence Synthetic peptide
KLIRVAP

SEQ ID NO: 34 - protein, 7 aa, Artificial Sequence
  Description of Artificial Sequence Synthetic peptide
KTLRIAP

SEQ ID NO: 35 - protein, 7 aa, Artificial Sequence
  Description of Artificial Sequence Synthetic peptide
KLLRIAP

SEQ ID NO: 36 - protein, 7 aa, Artificial Sequence
  Description of Artificial Sequence Synthetic peptide
KIIRTAP

SEQ ID NO: 37 - protein, 7 aa, Artificial Sequence
  Description of Artificial Sequence Synthetic peptide
ATWRIGP

SEQ ID NO: 38 - protein, 7 aa, Artificial Sequence
  Description of Artificial Sequence Synthetic peptide
KDVRIAP

SEQ ID NO: 39 - protein, 7 aa, Artificial Sequence
  Description of Artificial Sequence Synthetic peptide
KVVRIEP

SEQ ID NO: 40 - protein, 7 aa, Artificial Sequence
  Description of Artificial Sequence Synthetic peptide
KVVRTAP

SEQ ID NO: 41 - protein, 12 aa, Artificial Sequence
  Description of Artificial Sequence Synthetic peptide
KLLVPSETRGIW

SEQ ID NO: 42 - protein, 12 aa, Artificial Sequence
  Description of Artificial Sequence Synthetic peptide
SLLYSSEYSGIW

SEQ ID NO: 43 - protein, 12 aa, Artificial Sequence
  Description of Artificial Sequence Synthetic peptide
KLLSSNTYGIWM

SEQ ID NO: 44 - protein, 12 aa, Artificial Sequence
  Description of Artificial Sequence Synthetic peptide
KLLSSNTYGIWM

SEQ ID NO: 45 - protein, 12 aa, Artificial Sequence
  Description of Artificial Sequence Synthetic peptide
KLLSSNTYGIWM

SEQ ID NO: 46 - protein, 12 aa, Artificial Sequence
  Description of Artificial Sequence Synthetic peptide
KLLCSAPTQLWC

SEQ ID NO: 47 - protein, 12 aa, Artificial Sequence
  Description of Artificial Sequence Synthetic peptide
KLLGYTTSAGIW

SEQ ID NO: 48 - protein, 12 aa, Artificial Sequence
  Description of Artificial Sequence Synthetic peptide
LCYHRDGSYPTS

SEQ ID NO: 49 - protein, 12 aa, Artificial Sequence
  Description of Artificial Sequence Synthetic peptide
SLLKHSLSAGIW

SEQ ID NO: 50 - protein, 12 aa, Artificial Sequence
  Description of Artificial Sequence Synthetic peptide
SLLKHSLSAGIW

SEQ ID NO: 51 - protein, 7 aa, Artificial Sequence
  Description of Artificial Sequence Synthetic peptide
LPGLSKL

SEQ ID NO: 52 - protein, 7 aa, Artificial Sequence
  Description of Artificial Sequence Synthetic peptide
LGKLPNL

SEQ ID NO: 53 - protein, 7 aa, Artificial Sequence
  Description of Artificial Sequence Synthetic peptide
QGKLLPA

SEQ ID NO: 54 - protein, 7 aa, Artificial Sequence
  Description of Artificial Sequence Synthetic peptide
QGKLLPS

SEQ ID NO: 55 - protein, 7 aa, Artificial Sequence
  Description of Artificial Sequence Synthetic peptide
GGKLPEL

SEQ ID NO: 56 - protein, 7 aa, Artificial Sequence
  Description of Artificial Sequence Synthetic peptide
XGKIPEL

SEQ ID NO: 57 - protein, 7 aa, Artificial Sequence
  Description of Artificial Sequence Synthetic peptide
YGKLSLF

SEQ ID NO: 58 - protein, 7 aa, Artificial Sequence
  Description of Artificial Sequence Synthetic peptide
SHGKLLA

SEQ ID NO: 59 - protein, 7 aa, Artificial Sequence
  Description of Artificial Sequence Synthetic peptide
SYGKLLA

SEQ ID NO: 60 - protein, 7 aa, Artificial Sequence
  Description of Artificial Sequence Synthetic peptide
LGKLLPE

SEQ ID NO: 61 - protein, 7 aa, Artificial Sequence
  Description of Artificial Sequence Synthetic peptide
PLGKLVG

SEQ ID NO: 62 - protein, 7 aa, Artificial Sequence
  Description of Artificial Sequence Synthetic peptide
QGKMLPA

SEQ ID NO: 63 - protein, 7 aa, Artificial Sequence
  Description of Artificial Sequence Synthetic peptide
SCGKLVC

SEQ ID NO: 64 - protein, 7 aa, Artificial Sequence
  Description of Artificial Sequence Synthetic peptide
TGKLQCI

SEQ ID NO: 65 - protein, 7 aa, Artificial Sequence
  Description of Artificial Sequence Synthetic peptide
TFGKLLA

SEQ ID NO: 66 - protein, 12 aa, Artificial Sequence
  Description of Artificial Sequence Synthetic peptide
QHSWSCSGKLLC

SEQ ID NO: 67 - protein, 12 aa, Artificial Sequence
  Description of Artificial Sequence Synthetic peptide
SLWTLHGSLISA

SEQ ID NO: 68 - protein, 12 aa, Artificial Sequence
  Description of Artificial Sequence Synthetic peptide
SLWTLHGSLISA

SEQ ID NO: 69 - protein, 12 aa, Artificial Sequence
  Description of Artificial Sequence Synthetic peptide
SIWQTSGVLISY

SEQ ID NO: 70 - protein, 12 aa, Artificial Sequence
  Description of Artificial Sequence Synthetic peptide
GIWNHPSFLVWE

SEQ ID NO: 71 - protein, 12 aa, Artificial Sequence
  Description of Artificial Sequence Synthetic peptide
SHWSPXRSXRXX

SEQ ID NO: 72 - protein, 7 aa, Artificial Sequence
  Description of Artificial Sequence Synthetic peptide
NILSTLL

SEQ ID NO: 73 - protein, 7 aa, Artificial Sequence
  Description of Artificial Sequence Synthetic peptide
NVFNWKW

SEQ ID NO: 74 - protein, 7 aa, Artificial Sequence
  Description of Artificial Sequence Synthetic peptide
EFRWAWA

SEQ ID NO: 75 - protein, 7 aa, Artificial Sequence
  Description of Artificial Sequence Synthetic peptide
DWTWSWN

SEQ ID NO: 76 - protein, 7 aa, Artificial Sequence
  Description of Artificial Sequence Synthetic peptide
WSWGWMA

SEQ ID NO: 77 - protein, 12 aa, Artificial Sequence
  Description of Artificial Sequence Synthetic peptide
SLWTLHGSLISA

SEQ ID NO: 78 - protein, 12 aa, Artificial Sequence
  Description of Artificial Sequence Synthetic peptide
SIWQTSGVLISY

SEQ ID NO: 79 - protein, 12 aa, Artificial Sequence
  Description of Artificial Sequence Synthetic peptide
VPPSFLARWPAW

SEQ ID NO: 80 - protein, 7 aa, Artificial Sequence
  Description of Artificial Sequence Synthetic peptide
TSVKHGL

SEQ ID NO: 81 - protein, 7 aa, Artificial Sequence
  Description of Artificial Sequence Synthetic peptide
NPVKHGL

SEQ ID NO: 82 - protein, 7 aa, Artificial Sequence
  Description of Artificial Sequence Synthetic peptide
VCTHREC

SEQ ID NO: 83 - protein, 7 aa, Artificial Sequence
  Description of Artificial Sequence Synthetic peptide
LCTHREC

SEQ ID NO: 84 - protein, 7 aa, Artificial Sequence
  Description of Artificial Sequence Synthetic peptide
EKLWMLH

SEQ ID NO: 85 - protein, 18 aa, Artificial Sequence
  Description of Artificial Sequence Synthetic peptide
FNVTTGIRDKKQKVNALF

SEQ ID NO: 86 - protein, 18 aa, Artificial Sequence
  Description of Artificial Sequence Synthetic peptide
PNNNTRKSIRIGPGQAFY

SEQ ID NO: 87 - protein, 18 aa, Artificial Sequence
  Description of Artificial Sequence Synthetic peptide
RKSIRIGPGQAFYATGDI

SEQ ID NO: 88 - protein, 18 aa, Artificial Sequence
  Description of Artificial Sequence Synthetic peptide
YKVVEIKPLGIAPTKAKR

SEQ ID NO: 89 - protein, 18 aa, Artificial Sequence
  Description of Artificial Sequence Synthetic peptide
LAIERYLQDQQLLGIWGC

SEQ ID NO: 90 - protein, 18 aa, Human immunodeficiency virus
LGIWGCSGKLICTTAVPW

SEQ ID NO: 91 - protein, 18 aa, Artificial Sequence
  Description of Artificial Sequence Synthetic peptide
NEKDLLALDSWENLWNWF

SEQ ID NO: 92 - protein, 18 aa, Artificial Sequence
  Description of Artificial Sequence Synthetic peptide
NLWNWFSITKWLWYIKIF

SEQ ID NO: 93 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 94 - protein, 12 aa, Artificial Sequence
  Description of Artificial Sequence Synthetic peptide
TKWVHTGPGERH

SEQ ID NO: 95 - protein, 12 aa, Artificial Sequence
  Description of Artificial Sequence Synthetic peptide
MHKPIRTGPPEL

SEQ ID NO: 96 - protein, 12 aa, Artificial Sequence
  Description of Artificial Sequence Synthetic peptide
MSPPKHIRLGPN

SEQ ID NO: 97 - protein, 14 aa, Artificial Sequence
  Description of Artificial Sequence Synthetic peptide
HAKLIRTGPVAVHA

SEQ ID NO: 98 - protein, 12 aa, Artificial Sequence
  Description of Artificial Sequence Synthetic peptide
GLRPGRAQPFYA

SEQ ID NO: 99 - protein, 12 aa, Artificial Sequence
  Description of Artificial Sequence Synthetic peptide
MPRASPGSPHYT

SEQ ID NO: 100 - protein, 12 aa, Artificial Sequence
  Description of Artificial Sequence Synthetic peptide
GLRPGMAQPFYA

SEQ ID NO: 101 - protein, 12 aa, Artificial Sequence
  Description of Artificial Sequence Synthetic peptide
GLRPGMAQPFYP

SEQ ID NO: 102 - protein, 12 aa, Artificial Sequence
  Description of Artificial Sequence Synthetic peptide
TRPPPGWTAYVT

SEQ ID NO: 103 - protein, 12 aa, Artificial Sequence
  Description of Artificial Sequence Synthetic peptide
TSRWDDVRHSIT

SEQ ID NO: 104 - protein, 7 aa, Artificial Sequence
  Description of Artificial Sequence Synthetic peptide
KAIRIAP

SEQ ID NO: 105 - protein, 7 aa, Artificial Sequence
  Description of Artificial Sequence Synthetic peptide
KTIRMGP

SEQ ID NO: 106 - protein, 7 aa, Artificial Sequence
  Description of Artificial Sequence Synthetic peptide
KAIRVGP

SEQ ID NO: 107 - protein, 7 aa, Artificial Sequence
  Description of Artificial Sequence Synthetic peptide
KPLRLGP

SEQ ID NO: 108 - protein, 7 aa, Artificial Sequence
  Description of Artificial Sequence Synthetic peptide
IRLGPDQ

SEQ ID NO: 109 - protein, 7 aa, Artificial Sequence
  Description of Artificial Sequence Synthetic peptide
IRLGPGQ

SEQ ID NO: 110 - protein, 4 aa, Human immunodeficiency virus
KLIC

SEQ ID NO: 111 - DNA, 45 bp, Artificial Sequence
  Description of Artificial Sequence Synthetic primer
ggcccggggatcctaactttcaacagtttcggccgaacctccacc

SEQ ID NO: 112 - DNA, 48 bp, Artificial Sequence
  Description of Artificial Sequence Synthetic primer
cgcccgcggattaatggccctttagtggtacctttctattctcactct

SEQ ID NO: 113 - protein, 7 aa, Artificial Sequence
  Description of Artificial Sequence Synthetic peptide
TRKSIRI

SEQ ID NO: 114 - protein, 8 aa, Artificial Sequence
  Description of Artificial Sequence Synthetic peptide
GPGQAFYA

SEQ ID NO: 115 - protein, 7 aa, Artificial Sequence
  Description of Artificial Sequence Synthetic peptide
TRKSIHI

SEQ ID NO: 116 - protein, 8 aa, Artificial Sequence
  Description of Artificial Sequence Synthetic peptide
GPGRAFYT

SEQ ID NO: 117 - protein, 6 aa, Artificial Sequence
  Description of Artificial Sequence Synthetic peptide
GPSKIF

SEQ ID NO: 118 - protein, 6 aa, Artificial Sequence
  Description of Artificial Sequence Synthetic peptide
TWGWAF

SEQ ID NO: 119 - protein, 12 aa, Artificial Sequence
  Description of Artificial Sequence Synthetic peptide
MHKPIRTGPAEV

SEQ ID NO: 120 - protein, 12 aa, Artificial Sequence
  Description of Artificial Sequence Synthetic peptide
HAKLIRTGPVAV

SEQ ID NO: 121 - protein, 35 aa, Artificial Sequence
  Description of Artificial Sequence Synthetic Consensus C polypeptide sequence
CTRPNNNTRKSIRIGPGQTFYATGDIIGDIRQAHC

SEQ ID NO: 122 - protein, 35 aa, Artificial Sequence
  Description of Artificial Sequence Synthetic polypeptide
CTRPNNNTRKSIRIGPGQAFYATGDIIGDIRQAHC

SEQ ID NO: 123 - protein, 12 aa, Artificial Sequence
  Description of Artificial Sequence Synthetic peptide
TRPPPGSTAYVT

SEQ ID NO: 124 - protein, 12 aa, Artificial Sequence
  Description of Artificial Sequence Synthetic peptide
GLRPGMAQPFYA

SEQ ID NO: 125 - protein, 31 aa, Artificial Sequence
  Description of Artificial Sequence Synthetic polypeptide
RDNWRSELYKYKVVEIKPLGIAPTKAKRRVV

SEQ ID NO: 126 - protein, 31 aa, Artificial Sequence
  Description of Artificial Sequence Synthetic polypeptide
RNNWRSELYKYKVVEIKPLGIAPTKAKRRVV

SEQ ID NO: 127 - protein, 7 aa, Artificial Sequence
  Description of Artificial Sequence Synthetic peptide
KDVRIAP

SEQ ID NO: 128 - protein, 7 aa, Artificial Sequence
  Description of Artificial Sequence Synthetic peptide
KTLRIAP

SEQ ID NO: 129 - protein, 7 aa, Artificial Sequence
  Description of Artificial Sequence Synthetic peptide
KVVRIEP

SEQ ID NO: 130 - protein, 7 aa, Artificial Sequence
  Description of Artificial Sequence Synthetic peptide
KLLRIAP

SEQ ID NO: 131 - protein, 7 aa, Artificial Sequence
  Description of Artificial Sequence Synthetic peptide
ATWRIGP

SEQ ID NO: 132 - protein, 7 aa, Artificial Sequence
  Description of Artificial Sequence Synthetic peptide
TQVRDKQ

SEQ ID NO: 133 - protein, 7 aa, Artificial Sequence
  Description of Artificial Sequence Synthetic peptide
RDQNEPS

SEQ ID NO: 134 - protein, 7 aa, Artificial Sequence
  Description of Artificial Sequence Synthetic peptide
TMIRDKM

SEQ ID NO: 135 - protein, 7 aa, Artificial Sequence
  Description of Artificial Sequence Synthetic peptide
SLVRDKQ

SEQ ID NO: 136 - protein, 12 aa, Artificial Sequence
  Description of Artificial Sequence Synthetic peptide
IWKPIRMGPHLP

SEQ ID NO: 137 - protein, 12 aa, Artificial Sequence
  Description of Artificial Sequence Synthetic peptide
YRPGPAHANFTT

SEQ ID NO: 138 - protein, 12 aa, Artificial Sequence
  Description of Artificial Sequence Synthetic peptide
GLRPGMAQPFYA

SEQ ID NO: 139 - protein, 12 aa, Artificial Sequence
  Description of Artificial Sequence Synthetic peptide
IRPHPGHMYYSW

SEQ ID NO: 140 - protein, 12 aa, Artificial Sequence
  Description of Artificial Sequence Synthetic peptide
IRPAPGYGTYAL

SEQ ID NO: 141 - protein, 12 aa, Artificial Sequence
  Description of Artificial Sequence Synthetic peptide
NIRPAPGTAYYL

SEQ ID NO: 142 - protein, 12 aa, Artificial Sequence
  Description of Artificial Sequence Synthetic peptide
YRPGPTDAAYAK

SEQ ID NO: 143 - protein, 12 aa, Artificial Sequence
  Description of Artificial Sequence Synthetic peptide
TRPSPADPHYAS

SEQ ID NO: 144 - protein, 12 aa, Artificial Sequence
  Description of Artificial Sequence Synthetic peptide
ATRGGPATSHYA

SEQ ID NO: 145 - protein, 12 aa, Artificial Sequence
  Description of Artificial Sequence Synthetic peptide
GLRPGMAQPFYA

SEQ ID NO: 146 - protein, 12 aa, Artificial Sequence
  Description of Artificial Sequence Synthetic peptide
ARPAPGAAYYIL

SEQ ID NO: 147 - protein, 12 aa, Artificial Sequence
  Description of Artificial Sequence Synthetic peptide
IRPGPAITDYPR

SEQ ID NO: 148 - protein, 12 aa, Artificial Sequence
  Description of Artificial Sequence Synthetic peptide
ISGHGTTVTDDA

SEQ ID NO: 149 - protein, 7 aa, Artificial Sequence
  Description of Artificial Sequence Synthetic peptide
MKPGPHR

SEQ ID NO: 150 - protein, 7 aa, Artificial Sequence
  Description of Artificial Sequence Synthetic peptide
IRTGPGL

SEQ ID NO: 151 - protein, 12 aa, Artificial Sequence
  Description of Artificial Sequence Synthetic peptide
KLLGYTTSAGIW

SEQ ID NO: 152 - protein, 12 aa, Artificial Sequence
  Description of Artificial Sequence Synthetic peptide
KLLSPGDQYSLW

SEQ ID NO: 153 - protein, 12 aa, Artificial Sequence
  Description of Artificial Sequence Synthetic peptide
KLLSSNTYGIWM

SEQ ID NO: 154 - protein, 12 aa, Artificial Sequence
  Description of Artificial Sequence Synthetic peptide
TLLCSKCSSGVW

SEQ ID NO: 155 - protein, 12 aa, Artificial Sequence
  Description of Artificial Sequence Synthetic peptide
THQCLGKLQCGV

SEQ ID NO: 156 - protein, 7 aa, Artificial Sequence
  Description of Artificial Sequence Synthetic peptide
SHGKLLA

SEQ ID NO: 157 - protein, 7 aa, Artificial Sequence
  Description of Artificial Sequence Synthetic peptide
SHGKLQA

SEQ ID NO: 158 - protein, 7 aa, Artificial Sequence
  Description of Artificial Sequence Synthetic peptide
TLGKLIA

SEQ ID NO: 159 - protein, 7 aa, Artificial Sequence
  Description of Artificial Sequence Synthetic peptide
SGKLSCT

SEQ ID NO: 160 - protein, 7 aa, Artificial Sequence
  Description of Artificial Sequence Synthetic peptide
VHGKLLA

SEQ ID NO: 161 - protein, 7 aa, Artificial Sequence
  Description of Artificial Sequence Synthetic peptide
RGKLREL

SEQ ID NO: 162 - protein, 7 aa, Artificial Sequence
  Description of Artificial Sequence Synthetic peptide
MRGKLVA

SEQ ID NO: 163 - protein, 7 aa, Artificial Sequence
  Description of Artificial Sequence Synthetic peptide
TGGKLVT

SEQ ID NO: 164 - protein, 7 aa, Artificial Sequence
  Description of Artificial Sequence Synthetic peptide
SYGKLAA

SEQ ID NO: 165 - protein, 7 aa, Artificial Sequence
  Description of Artificial Sequence Synthetic peptide
YGKMLPQ

SEQ ID NO: 166 - protein, 7 aa, Artificial Sequence
  Description of Artificial Sequence Synthetic peptide
LGKNLPT

SEQ ID NO: 167 - protein, 7 aa, Artificial Sequence
  Description of Artificial Sequence Synthetic peptide
EYGKLAA

SEQ ID NO: 168 - protein, 7 aa, Artificial Sequence
  Description of Artificial Sequence Synthetic peptide
IHGKLAA

SEQ ID NO: 169 - protein, 7 aa, Artificial Sequence
  Description of Artificial Sequence Synthetic peptide
NSGKLVA

SEQ ID NO: 170 - protein, 7 aa, Artificial Sequence
  Description of Artificial Sequence Synthetic peptide
TGKLQCI

SEQ ID NO: 171 - protein, 12 aa, Artificial Sequence
  Description of Artificial Sequence Synthetic peptide
YLPIWMHPQRLI

SEQ ID NO: 172 - protein, 7 aa, Artificial Sequence
  Description of Artificial Sequence Synthetic peptide
SGRLPEL

SEQ ID NO: 173 - protein, 7 aa, Artificial Sequence
  Description of Artificial Sequence Synthetic peptide
QGKLLPS

SEQ ID NO: 174 - protein, 7 aa, Artificial Sequence
  Description of Artificial Sequence Synthetic peptide
LGKLLPE

SEQ ID NO: 175 - protein, 7 aa, Artificial Sequence
  Description of Artificial Sequence Synthetic peptide
QHGKLLS

SEQ ID NO: 176 - protein, 7 aa, Artificial Sequence
  Description of Artificial Sequence Synthetic peptide
YGKLHPD

SEQ ID NO: 177 - protein, 7 aa, Artificial Sequence
  Description of Artificial Sequence Synthetic peptide
THGKLTA

SEQ ID NO: 178 - protein, 7 aa, Artificial Sequence
  Description of Artificial Sequence Synthetic peptide
LGKNMPA

SEQ ID NO: 179 - protein, 12 aa, Artificial Sequence
  Description of Artificial Sequence Synthetic peptide
KISSRAWHEIAN

SEQ ID NO: 180 - protein, 7 aa, Artificial Sequence
  Description of Artificial Sequence Synthetic peptide
MGETQAV

SEQ ID NO: 181 - protein, 7 aa, Artificial Sequence
  Description of Artificial Sequence Synthetic peptide
APTDMHL

SEQ ID NO: 182 - protein, 12 aa, Artificial Sequence
  Description of Artificial Sequence Synthetic peptide
NTSFNWRWMDQE

SEQ ID NO: 183 - protein, 12 aa, Artificial Sequence
  Description of Artificial Sequence Synthetic peptide
RPPPGTDLYARQ

SEQ ID NO: 184 - protein, 12 aa, Artificial Sequence
  Description of Artificial Sequence Synthetic peptide
TKWVHTGPGERH

SEQ ID NO: 185 - protein, 12 aa, Artificial Sequence
  Description of Artificial Sequence Synthetic peptide
MHKPIRTGPAEV

SEQ ID NO: 186 - protein, 12 aa, Artificial Sequence
  Description of Artificial Sequence Synthetic peptide
MSPPKHIRLGPN

SEQ ID NO: 187 - protein, 12 aa, Artificial Sequence
  Description of Artificial Sequence Synthetic peptide
HAKLIRTGPVAV

SEQ ID NO: 188 - protein, 12 aa, Artificial Sequence
  Description of Artificial Sequence Synthetic peptide
GLRPGMAQPFYA

SEQ ID NO: 189 - protein, 12 aa, Artificial Sequence
  Description of Artificial Sequence Synthetic peptide
MPRASPGSPHYT

SEQ ID NO: 190 - protein, 12 aa, Artificial Sequence
  Description of Artificial Sequence Synthetic peptide
TRPPPGSTAYVT

SEQ ID NO: 191 - protein, 12 aa, Artificial Sequence
  Description of Artificial Sequence Synthetic peptide
TSRWDDVRHSIT

SEQ ID NO: 192 - protein, 12 aa, Artificial Sequence
  Description of Artificial Sequence Synthetic peptide
TLTKVPRLAPGV

SEQ ID NO: 193 - protein, 7 aa, Artificial Sequence
  Description of Artificial Sequence Synthetic peptide
KAIRIAP

SEQ ID NO: 194 - protein, 7 aa, Artificial Sequence
  Description of Artificial Sequence Synthetic peptide
KAIRVGP

SEQ ID NO: 195 - protein, 7 aa, Artificial Sequence
  Description of Artificial Sequence Synthetic peptide
KPLRLGP

SEQ ID NO: 196 - protein, 7 aa, Artificial Sequence
  Description of Artificial Sequence Synthetic peptide
IRLGPGQ

SEQ ID NO: 197 - protein, 12 aa, Artificial Sequence
  Description of Artificial Sequence Synthetic peptide
VRLPPGASGYTP

SEQ ID NO: 198 - protein, 7 aa, Artificial Sequence
  Description of Artificial Sequence Synthetic peptide
SCGKLYC

SEQ ID NO: 199 - protein, 7 aa, Artificial Sequence
  Description of Artificial Sequence Synthetic peptide
SHGKLLA

SEQ ID NO: 200 - protein, 7 aa, Artificial Sequence
  Description of Artificial Sequence Synthetic peptide
SHGKLLC

SEQ ID NO: 201 - protein, 12 aa, Artificial Sequence
  Description of Artificial Sequence Synthetic peptide
ASCGKMICTPLL

SEQ ID NO: 202 - protein, 12 aa, Artificial Sequence
  Description of Artificial Sequence Synthetic peptide
QACLGKLNCIVA

SEQ ID NO: 203 - protein, 12 aa, Artificial Sequence
  Description of Artificial Sequence Synthetic peptide
SSCMGRLSCIAL

SEQ ID NO: 204 - protein, 7 aa, Artificial Sequence
  Description of Artificial Sequence Synthetic peptide
TGKLQCI

SEQ ID NO: 205 - protein, 7 aa, Artificial Sequence
  Description of Artificial Sequence Synthetic peptide
DDFYWWL

SEQ ID NO: 206 - protein, 7 aa, Artificial Sequence
  Description of Artificial Sequence Synthetic peptide
DDILSTY

SEQ ID NO: 207 - protein, 7 aa, Artificial Sequence
  Description of Artificial Sequence Synthetic peptide
DDFRVPH

SEQ ID NO: 208 - protein, 12 aa, Artificial Sequence
  Description of Artificial Sequence Synthetic peptide
DDIIPRHDSHLT

SEQ ID NO: 209 - protein, 12 aa, Artificial Sequence
  Description of Artificial Sequence Synthetic peptide
FIDDVQYSVTSG

SEQ ID NO: 210 - protein, 7 aa, Artificial Sequence
  Description of Artificial Sequence Synthetic peptide
IRATPPY

SEQ ID NO: 211 - protein, 7 aa, Artificial Sequence
  Description of Artificial Sequence Synthetic peptide
IRATPPV

SEQ ID NO: 212 - protein, 7 aa, Artificial Sequence
  Description of Artificial Sequence Synthetic peptide
IRATPPP

SEQ ID NO: 213 - protein, 7 aa, Artificial Sequence
  Description of Artificial Sequence Synthetic peptide
IRATPPT

SEQ ID NO: 214 - protein, 7 aa, Artificial Sequence
  Description of Artificial Sequence Synthetic peptide
IRATPAF

SEQ ID NO: 215 - protein, 7 aa, Artificial Sequence
  Description of Artificial Sequence Synthetic peptide
IRATPTL

SEQ ID NO: 216 - protein, 12 aa, Artificial Sequence
  Description of Artificial Sequence Synthetic peptide
VQIPLYAHHPVI

SEQ ID NO: 217 - protein, 12 aa, Artificial Sequence
  Description of Artificial Sequence Synthetic peptide
SWDNEYVHWKAP

SEQ ID NO: 218 - protein, 7 aa, Artificial Sequence
  Description of Artificial Sequence Synthetic peptide
SKPLFVT

SEQ ID NO: 219 - protein, 7 aa, Artificial Sequence
  Description of Artificial Sequence Synthetic peptide
SYDNDNW

SEQ ID NO: 220 - protein, 7 aa, Artificial Sequence
  Description of Artificial Sequence Synthetic peptide
SADNDEL

SEQ ID NO: 221 - protein, 7 aa, Artificial Sequence
  Description of Artificial Sequence Synthetic peptide
QEMRMAW

SEQ ID NO: 222 - protein, 7 aa, Artificial Sequence
  Description of Artificial Sequence Synthetic peptide
LGKNMPW

SEQ ID NO: 223 - protein, 7 aa, Artificial Sequence
  Description of Artificial Sequence Synthetic peptide
YGKSYPL

SEQ ID NO: 224 - protein, 7 aa, Artificial Sequence
  Description of Artificial Sequence Synthetic peptide
MGKMPHL

SEQ ID NO: 225 - protein, 7 aa, Artificial Sequence
  Description of Artificial Sequence Synthetic peptide
SGKLPLL

SEQ ID NO: 226 - protein, 7 aa, Artificial Sequence
  Description of Artificial Sequence Synthetic peptide
YGKMTPM

SEQ ID NO: 227 - protein, 7 aa, Artificial Sequence
  Description of Artificial Sequence Synthetic peptide
YGKMTLL

SEQ ID NO: 228 - protein, 7 aa, Artificial Sequence
  Description of Artificial Sequence Synthetic peptide
YGKLPWI

SEQ ID NO: 229 - protein, 7 aa, Artificial Sequence
  Description of Artificial Sequence Synthetic peptide
KGKPAPI

SEQ ID NO: 230 - protein, 7 aa, Artificial Sequence
  Description of Artificial Sequence Synthetic peptide
FGKLHPE

SEQ ID NO: 231 - protein, 7 aa, Artificial Sequence
  Description of Artificial Sequence Synthetic peptide
FGKLPLL

SEQ ID NO: 232 - protein, 12 aa, Artificial Sequence
  Description of Artificial Sequence Synthetic peptide
QCVGKLLCDARR

SEQ ID NO: 233 - protein, 12 aa, Artificial Sequence
  Description of Artificial Sequence Synthetic peptide
QVNTCLGRMICT

SEQ ID NO: 234 - protein, 12 aa, Artificial Sequence
  Description of Artificial Sequence Synthetic peptide
ASCGKMICTPLL

SEQ ID NO: 235 - protein, 12 aa, Artificial Sequence
  Description of Artificial Sequence Synthetic peptide
SVRQMPIWLMSG

SEQ ID NO: 236 - protein, 12 aa, Artificial Sequence
  Description of Artificial Sequence Synthetic peptide
QSPGKLVALPGF

SEQ ID NO: 237 - protein, 7 aa, Artificial Sequence
  Description of Artificial Sequence Synthetic peptide
QGKPSPV

SEQ ID NO: 238 - protein, 12 aa, Artificial Sequence
  Description of Artificial Sequence Synthetic peptide
HCGKLICHAFSS

SEQ ID NO: 239 - protein, 12 aa, Artificial Sequence
  Description of Artificial Sequence Synthetic peptide
LIAPATPRGGDS

SEQ ID NO: 240 - protein, 12 aa, Artificial Sequence
  Description of Artificial Sequence Synthetic peptide
GSTPRGADALVR

SEQ ID NO: 241 - protein, 12 aa, Artificial Sequence
  Description of Artificial Sequence Synthetic peptide
HHWNRASALTPR

SEQ ID NO: 242 - protein, 7 aa, Artificial Sequence
  Description of Artificial Sequence Synthetic peptide
SHPHTPR

SEQ ID NO: 243 - protein, 12 aa, Artificial Sequence
  Description of Artificial Sequence Synthetic peptide
VRLPPGASGYTP

SEQ ID NO: 244 - protein, 12 aa, Artificial Sequence
  Description of Artificial Sequence Synthetic peptide
SCVGKHVCAHPL

SEQ ID NO: 245 - protein, 7 aa, Artificial Sequence
  Description of Artificial Sequence Synthetic peptide
CIGKYLC

SEQ ID NO: 246 - protein, 7 aa, Artificial Sequence
  Description of Artificial Sequence Synthetic peptide
LGQLRCT

SEQ ID NO: 247 - protein, 7 aa, Artificial Sequence
  Description of Artificial Sequence Synthetic peptide
LGKPPWL

SEQ ID NO: 248 - protein, 7 aa, Artificial Sequence
  Description of Artificial Sequence Synthetic peptide
TGGKLRA

SEQ ID NO: 249 - protein, 7 aa, Artificial Sequence
  Description of Artificial Sequence Synthetic peptide
MGKFYHA

SEQ ID NO: 250 - protein, 7 aa, Artificial Sequence
  Description of Artificial Sequence Synthetic peptide
PLGKLVG

SEQ ID NO: 251 - protein, 7 aa, Artificial Sequence
  Description of Artificial Sequence Synthetic peptide
TGKLQCI

SEQ ID NO: 252 - protein, 7 aa, Artificial Sequence
  Description of Artificial Sequence Synthetic peptide
PFGKLLS

SEQ ID NO: 253 - protein, 7 aa, Artificial Sequence
  Description of Artificial Sequence Synthetic peptide
PLGKLVG

SEQ ID NO: 254 - protein, 12 aa, Artificial Sequence
  Description of Artificial Sequence Synthetic peptide
TYLGKFPGIFRD

SEQ ID NO: 255 - protein, 12 aa, Artificial Sequence
  Description of Artificial Sequence Synthetic peptide
DTHPRCNKLHCW

SEQ ID NO: 256 - protein, 7 aa, Artificial Sequence
  Description of Artificial Sequence Synthetic peptide
SHGKLLA

SEQ ID NO: 257 - protein, 12 aa, Artificial Sequence
  Description of Artificial Sequence Synthetic peptide
GLRPGMAQPFYA

SEQ ID NO: 258 - protein, 12 aa, Artificial Sequence
  Description of Artificial Sequence Synthetic peptide
MPRASPGSPHYT

SEQ ID NO: 259 - protein, 12 aa, Artificial Sequence
  Description of Artificial Sequence Synthetic peptide
NCHRRGSRAFHY

SEQ ID NO: 260 - protein, 12 aa, Artificial Sequence
  Description of Artificial Sequence Synthetic peptide
TQLRAGPAQKFY

SEQ ID NO: 261 - protein, 12 aa, Artificial Sequence
  Description of Artificial Sequence Synthetic peptide
MHKPIRTGPAEV

SEQ ID NO: 262 - protein, 12 aa, Artificial Sequence
  Description of Artificial Sequence Synthetic peptide
DKLMMYYQPPGM

SEQ ID NO: 263 - protein, 12 aa, Artificial Sequence
  Description of Artificial Sequence Synthetic peptide
HLQNITGIKVPP

SEQ ID NO: 264 - protein, 12 aa, Artificial Sequence
  Description of Artificial Sequence Synthetic peptide
SCMGKLMCPSER

SEQ ID NO: 265 - protein, 12 aa, Artificial Sequence
  Description of Artificial Sequence Synthetic peptide
LEICNGKLNCII

SEQ ID NO: 266 - protein, 12 aa, Artificial Sequence
  Description of Artificial Sequence Synthetic peptide
NVLNNCMGKWVC

SEQ ID NO: 267 - protein, 12 aa, Artificial Sequence
  Description of Artificial Sequence Synthetic peptide
SPPVCNGKLLCT

SEQ ID NO: 268 - protein, 12 aa, Artificial Sequence
  Description of Artificial Sequence Synthetic peptide
EPSKPCAGKLLC

SEQ ID NO: 269 - protein, 12 aa, Artificial Sequence
  Description of Artificial Sequence Synthetic peptide
WCAGKHTCVTHS

SEQ ID NO: 270 - protein, 12 aa, Artificial Sequence
  Description of Artificial Sequence Synthetic peptide
GWSSCGGKLTCS

SEQ ID NO: 271 - protein, 12 aa, Artificial Sequence
  Description of Artificial Sequence Synthetic peptide
FGACQLGKLSAC

SEQ ID NO: 272 - protein, 12 aa, Artificial Sequence
  Description of Artificial Sequence Synthetic peptide
SMDPCYGKLNCL

SEQ ID NO: 273 - protein, 12 aa, Artificial Sequence
  Description of Artificial Sequence Synthetic peptide
YYERCLGKMQCA

SEQ ID NO: 274 - protein, 12 aa, Artificial Sequence
  Description of Artificial Sequence Synthetic peptide
CNKFLCSSDAAP

SEQ ID NO: 275 - protein, 12 aa, Artificial Sequence
  Description of Artificial Sequence Synthetic peptide
CNKMLCLDSMSP

SEQ ID NO: 276 - protein, 12 aa, Artificial Sequence
  Description of Artificial Sequence Synthetic peptide
GKPISLAPQFWT

SEQ ID NO: 277 - protein, 12 aa, Artificial Sequence
  Description of Artificial Sequence Synthetic peptide
QTACNKLLCLAT

SEQ ID NO: 278 - protein, 12 aa, Artificial Sequence
  Description of Artificial Sequence Synthetic peptide
NCLGHLRCTLGN

SEQ ID NO: 279 - protein, 12 aa, Artificial Sequence
  Description of Artificial Sequence Synthetic peptide
HACTGKLRCTTT

SEQ ID NO: 280 - protein, 12 aa, Artificial Sequence
  Description of Artificial Sequence Synthetic peptide
HCAGSLRCIHPP

SEQ ID NO: 281 - protein, 12 aa, Artificial Sequence
  Description of Artificial Sequence Synthetic peptide
EKLITSYQQARP

SEQ ID NO: 282 - protein, 12 aa, Artificial Sequence
  Description of Artificial Sequence Synthetic peptide
EKLITLSSHHFH

SEQ ID NO: 283 - protein, 12 aa, Artificial Sequence
  Description of Artificial Sequence Synthetic peptide
ELSCHKLLCTAQ

SEQ ID NO: 284 - protein, 6 aa, Artificial Sequence
  Description of Artificial Sequence Synthetic peptide
SCKLYC

SEQ ID NO: 285 - protein, 6 aa, Artificial Sequence
  Description of Artificial Sequence Synthetic peptide
SCKLVC

SEQ ID NO: 286 - protein, 7 aa, Artificial Sequence
  Description of Artificial Sequence Synthetic peptide
EKLISLY

SEQ ID NO: 287 - protein, 7 aa, Artificial Sequence
  Description of Artificial Sequence Synthetic peptide
EKLISFH

SEQ ID NO: 288 - protein, 7 aa, Artificial Sequence
  Description of Artificial Sequence Synthetic peptide
EKLIMFH

SEQ ID NO: 289 - protein, 7 aa, Artificial Sequence
  Description of Artificial Sequence Synthetic peptide
TGKLQCI

SEQ ID NO: 290 - protein, 7 aa, Artificial Sequence
  Description of Artificial Sequence Synthetic peptide
TGKLMCP

SEQ ID NO: 291 - protein, 7 aa, Artificial Sequence
  Description of Artificial Sequence Synthetic peptide
TGGKLLT

SEQ ID NO: 292 - protein, 7 aa, Artificial Sequence
  Description of Artificial Sequence Synthetic peptide
TLGKLAA

SEQ ID NO: 293 - protein, 7 aa, Artificial Sequence
  Description of Artificial Sequence Synthetic peptide
ACLGKLH

SEQ ID NO: 294 - protein, 7 aa, Artificial Sequence
  Description of Artificial Sequence Synthetic peptide
PPKGTKT

SEQ ID NO: 295 - protein, 7 aa, Artificial Sequence
  Description of Artificial Sequence Synthetic peptide
PYGKLTA

SEQ ID NO: 296 - protein, 7 aa, Artificial Sequence
  Description of Artificial Sequence Synthetic peptide
PHGKLTA

SEQ ID NO: 297 - protein, 7 aa, Artificial Sequence
  Description of Artificial Sequence Synthetic peptide
THGKLHA

SEQ ID NO: 298 - protein, 7 aa, Artificial Sequence
  Description of Artificial Sequence Synthetic peptide
MHGKLRA

SEQ ID NO: 299 - protein, 7 aa, Artificial Sequence
  Description of Artificial Sequence Synthetic peptide
SYGKLLA

SEQ ID NO: 300 - protein, 7 aa, Artificial Sequence
  Description of Artificial Sequence Synthetic peptide
YGKLLCG

SEQ ID NO: 301 - protein, 7 aa, Artificial Sequence
  Description of Artificial Sequence Synthetic peptide
YAGKLLA

SEQ ID NO: 302 - protein, 7 aa, Artificial Sequence
  Description of Artificial Sequence Synthetic peptide
SHGKLLA

SEQ ID NO: 303 - protein, 7 aa, Artificial Sequence
  Description of Artificial Sequence Synthetic peptide
LGRLSCM

SEQ ID NO: 304 - protein, 7 aa, Artificial Sequence
  Description of Artificial Sequence Synthetic peptide
FGKMLPE

SEQ ID NO: 305 - protein, 7 aa, Artificial Sequence
  Description of Artificial Sequence Synthetic peptide
NFGKLSA

SEQ ID NO: 306 - protein, 7 aa, Artificial Sequence
  Description of Artificial Sequence Synthetic peptide
NFGKLRA

SEQ ID NO: 307 - protein, 7 aa, Artificial Sequence
  Description of Artificial Sequence Synthetic peptide
WQGKLIA

SEQ ID NO: 308 - protein, 7 aa, Artificial Sequence
  Description of Artificial Sequence Synthetic peptide
LSGKLIA

SEQ ID NO: 309 - protein, 7 aa, Artificial Sequence
  Description of Artificial Sequence Synthetic peptide
KFGKLSA

SEQ ID NO: 310 - protein, 12 aa, Artificial Sequence
  Description of Artificial Sequence Synthetic peptide
KLLSPGDQYSLW

SEQ ID NO: 311 - protein, 12 aa, Artificial Sequence
  Description of Artificial Sequence Synthetic peptide
LQKETPLTWPPP

SEQ ID NO: 312 - protein, 12 aa, Artificial Sequence
  Description of Artificial Sequence Synthetic peptide
SVSVGMKPSPRP

SEQ ID NO: 313 - protein, 12 aa, Artificial Sequence
  Description of Artificial Sequence Synthetic peptide
KVPQKKPRSTPN

SEQ ID NO: 314 - protein, 12 aa, Artificial Sequence
  Description of Artificial Sequence Synthetic peptide
HCGGSPGSPHHP

SEQ ID NO: 315 - protein, 7 aa, Artificial Sequence
  Description of Artificial Sequence Synthetic peptide
ITRNFLL

SEQ ID NO: 316 - protein, 7 aa, Artificial Sequence
  Description of Artificial Sequence Synthetic peptide
PWSSFFP

SEQ ID NO: 317 - protein, 12 aa, Artificial Sequence
  Description of Artificial Sequence Synthetic peptide
QLSHSTWSPAGM

SEQ ID NO: 318 - protein, 12 aa, Artificial Sequence
  Description of Artificial Sequence Synthetic peptide
SSQQWALRSLSH

SEQ ID NO: 319 - protein, 12 aa, Artificial Sequence
  Description of Artificial Sequence Synthetic peptide
TYWPSWFLSHPP

SEQ ID NO: 320 - protein, 7 aa, Artificial Sequence
  Description of Artificial Sequence Synthetic peptide
LSHVPPP

SEQ ID NO: 321 - protein, 7 aa, Artificial Sequence
  Description of Artificial Sequence Synthetic peptide
LSQVPPP

SEQ ID NO: 322 - protein, 7 aa, Artificial Sequence
  Description of Artificial Sequence Synthetic peptide
LSHVPPP

SEQ ID NO: 323 - protein, 7 aa, Artificial Sequence
  Description of Artificial Sequence Synthetic peptide
FTSVPRP

SEQ ID NO: 324 - protein, 7 aa, Artificial Sequence
  Description of Artificial Sequence Synthetic peptide
SSPFVPW

SEQ ID NO: 325 - protein, 7 aa, Artificial Sequence
  Description of Artificial Sequence Synthetic peptide
SLAPPWL

SEQ ID NO: 326 - protein, 7 aa, Artificial Sequence
  Description of Artificial Sequence Synthetic peptide
PPMAIHI

SEQ ID NO: 327 - protein, 7 aa, Artificial Sequence
  Description of Artificial Sequence Synthetic peptide
PPNHTQP

SEQ ID NO: 328 - protein, 7 aa, Artificial Sequence
  Description of Artificial Sequence Synthetic peptide
NQAPSPM

SEQ ID NO: 329 - protein, 7 aa, Artificial Sequence
  Description of Artificial Sequence Synthetic peptide
PSRKPSQ

SEQ ID NO: 330 - protein, 7 aa, Artificial Sequence
  Description of Artificial Sequence Synthetic peptide
PSPKLQN

SEQ ID NO: 331 - protein, 12 aa, Artificial Sequence
  Description of Artificial Sequence Synthetic peptide
YTSSSRTVSPQL

SEQ ID NO: 332 - protein, 7 aa, Artificial Sequence
  Description of Artificial Sequence Synthetic peptide
YTDYSPR

SEQ ID NO: 333 - protein, 7 aa, Artificial Sequence
  Description of Artificial Sequence Synthetic peptide
PRFYSAV

SEQ ID NO: 334 - protein, 12 aa, Artificial Sequence
  Description of Artificial Sequence Synthetic peptide
FTPIYQIYSNAP

SEQ ID NO: 335 - protein, 12 aa, Artificial Sequence
  Description of Artificial Sequence Synthetic peptide
LHKTNNQLAAAL

SEQ ID NO: 336 - protein, 7 aa, Artificial Sequence
  Description of Artificial Sequence Synthetic peptide
LAASFNA

SEQ ID NO: 337 - protein, 7 aa, Artificial Sequence
  Description of Artificial Sequence Synthetic peptide
MTAARTP

SEQ ID NO: 338 - protein, 12 aa, Artificial Sequence
  Description of Artificial Sequence Synthetic peptide
AAHLNKAPKMAF

SEQ ID NO: 339 - protein, 7 aa, Artificial Sequence
  Description of Artificial Sequence Synthetic peptide
FASAKIP

SEQ ID NO: 340 - protein, 12 aa, Artificial Sequence
  Description of Artificial Sequence Synthetic peptide
AQLFALPHKASF

SEQ ID NO: 341 - protein, 12 aa, Artificial Sequence
  Description of Artificial Sequence Synthetic peptide
SGHMHKMSRTTL

SEQ ID NO: 342 - protein, 12 aa, Artificial Sequence
  Description of Artificial Sequence Synthetic peptide
NWWKAMPITLQS

SEQ ID NO: 343 - protein, 12 aa, Artificial Sequence
  Description of Artificial Sequence Synthetic peptide
HWPVTLGRWATV

SEQ ID NO: 344 - protein, 7 aa, Artificial Sequence
  Description of Artificial Sequence Synthetic peptide
KAPHTTS

SEQ ID NO: 345 - protein, 7 aa, Artificial Sequence
  Description of Artificial Sequence Synthetic peptide
GWNDLQH

SEQ ID NO: 346 - protein, 7 aa, Artificial Sequence
  Description of Artificial Sequence Synthetic peptide
IVSRPDL

SEQ ID NO: 347 - protein, 12 aa, Artificial Sequence
  Description of Artificial Sequence Synthetic peptide
TIRDYLNVMLNL

SEQ ID NO: 348 - protein, 12 aa, Artificial Sequence
  Description of Artificial Sequence Synthetic peptide
TIGYPLNRWPLM

SEQ ID NO: 349 - protein, 7 aa, Artificial Sequence
  Description of Artificial Sequence Synthetic peptide
MAYYDIA

SEQ ID NO: 350 - protein, 7 aa, Artificial Sequence
  Description of Artificial Sequence Synthetic peptide
YHKSTDN

SEQ ID NO: 351 - protein, 12 aa, Artificial Sequence
  Description of Artificial Sequence Synthetic peptide
TSRAPHISAHTN

SEQ ID NO: 352 - protein, 7 aa, Artificial Sequence
  Description of Artificial Sequence Synthetic peptide
PMWEKCD

SEQ ID NO: 353 - protein, 7 aa, Artificial Sequence
  Description of Artificial Sequence Synthetic peptide
FSVPRYY

SEQ ID NO: 354 - protein, 7 aa, Artificial Sequence
  Description of Artificial Sequence Synthetic peptide
QQDQPNR

SEQ ID NO: 355 - protein, 7 aa, Artificial Sequence
  Description of Artificial Sequence Synthetic peptide
STQTLAR

SEQ ID NO: 356 - protein, 7 aa, Artificial Sequence
  Description of Artificial Sequence Synthetic peptide
PPTPSTR

SEQ ID NO: 357 - protein, 7 aa, Artificial Sequence
  Description of Artificial Sequence Synthetic peptide
TQIYLDA

SEQ ID NO: 358 - protein, 7 aa, Artificial Sequence
  Description of Artificial Sequence Synthetic peptide
EATKLKP

SEQ ID NO: 359 - protein, 7 aa, Artificial Sequence
  Description of Artificial Sequence Synthetic peptide
PAHPRSQ

SEQ ID NO: 360 - protein, 7 aa, Artificial Sequence
  Description of Artificial Sequence Synthetic peptide
SETSQLP

SEQ ID NO: 361 - protein, 7 aa, Artificial Sequence
  Description of Artificial Sequence Synthetic peptide
MAAQVTG

SEQ ID NO: 362 - protein, 7 aa, Artificial Sequence
  Description of Artificial Sequence Synthetic peptide
TPLLPSP

SEQ ID NO: 363 - protein, 7 aa, Artificial Sequence
  Description of Artificial Sequence Synthetic peptide
QYYTKIK

SEQ ID NO: 364 - protein, 7 aa, Artificial Sequence
  Description of Artificial Sequence Synthetic peptide
QLSQTPP

SEQ ID NO: 365 - protein, 7 aa, Artificial Sequence
  Description of Artificial Sequence Synthetic peptide
MQLRPST

SEQ ID NO: 366 - protein, 7 aa, Artificial Sequence
  Description of Artificial Sequence Synthetic peptide
LSHVPPP

SEQ ID NO: 367 - protein, 14 aa, Artificial Sequence
  Description of Artificial Sequence Synthetic peptide
SSPFVPWSLAPPWL

SEQ ID NO: 368 - protein, 14 aa, Artificial Sequence
  Description of Artificial Sequence Synthetic peptide
SYSLSRLQPTTWWW

SEQ ID NO: 369 - protein, 12 aa, Artificial Sequence
  Description of Artificial Sequence Synthetic peptide
SWHHKDQHHPNR

SEQ ID NO: 370 - protein, 12 aa, Artificial Sequence
  Description of Artificial Sequence Synthetic peptide
VGTDFNYHESNM

SEQ ID NO: 371 - protein, 12 aa, Artificial Sequence
  Description of Artificial Sequence Synthetic peptide
TTSNKTFPWDFV

SEQ ID NO: 372 - protein, 12 aa, Artificial Sequence
  Description of Artificial Sequence Synthetic peptide
DASGPSNFPKWK

SEQ ID NO: 373 - protein, 12 aa, Artificial Sequence
  Description of Artificial Sequence Synthetic peptide
YTTTTYSPILGR

SEQ ID NO: 374 - protein, 12 aa, Artificial Sequence
  Description of Artificial Sequence Synthetic peptide
WASHYSNRHPPL

SEQ ID NO: 375 - protein, 12 aa, Artificial Sequence
  Description of Artificial Sequence Synthetic peptide
NTFDNQNNTSKT

SEQ ID NO: 376 - protein, 12 aa, Artificial Sequence
  Description of Artificial Sequence Synthetic peptide
HANTPNSAPDKT

SEQ ID NO: 377 - protein, 12 aa, Artificial Sequence
  Description of Artificial Sequence Synthetic peptide
VHAPRGADFLGI

SEQ ID NO: 378 - protein, 12 aa, Artificial Sequence
  Description of Artificial Sequence Synthetic peptide
SPSPNWGNSTPR

SEQ ID NO: 379 - protein, 12 aa, Artificial Sequence
  Description of Artificial Sequence Synthetic peptide
TDINTPRGPDMW

SEQ ID NO: 380 - protein, 12 aa, Artificial Sequence
  Description of Artificial Sequence Synthetic peptide
YTKSTPRGPDIW

SEQ ID NO: 381 - protein, 12 aa, Artificial Sequence
  Description of Artificial Sequence Synthetic peptide
IVITNTPRGPDF

SEQ ID NO: 382 - protein, 12 aa, Artificial Sequence
  Description of Artificial Sequence Synthetic peptide
WSTPRGPDTGDR

SEQ ID NO: 383 - protein, 12 aa, Artificial Sequence
  Description of Artificial Sequence Synthetic peptide
HLHTPRGVDNFL

SEQ ID NO: 384 - protein, 12 aa, Artificial Sequence
  Description of Artificial Sequence Synthetic peptide
TDINTPRGPDMW

SEQ ID NO: 385 - protein, 12 aa, Artificial Sequence
  Description of Artificial Sequence Synthetic peptide
FQSHWKPQPSPR

SEQ ID NO: 386 - protein, 12 aa, Artificial Sequence
  Description of Artificial Sequence Synthetic peptide
QWLTPRGADSLH

SEQ ID NO: 387 - protein, 12 aa, Artificial Sequence
  Description of Artificial Sequence Synthetic peptide
NYWIMTPRGPDS

SEQ ID NO: 388 - protein, 12 aa, Artificial Sequence
  Description of Artificial Sequence Synthetic peptide
YWPHHTRLETPK

SEQ ID NO: 389 - protein, 7 aa, Artificial Sequence
  Description of Artificial Sequence Synthetic peptide
TVFYAPR

SEQ ID NO: 390 - protein, 7 aa, Artificial Sequence
  Description of Artificial Sequence Synthetic peptide
KTYLTPR

SEQ ID NO: 391 - protein, 7 aa, Artificial Sequence
  Description of Artificial Sequence Synthetic peptide
FYGTTPR

SEQ ID NO: 392 - protein, 7 aa, Artificial Sequence
  Description of Artificial Sequence Synthetic peptide
SMWFTPR

SEQ ID NO: 393 - protein, 7 aa, Artificial Sequence
  Description of Artificial Sequence Synthetic peptide
FDLTTPR

SEQ ID NO: 394 - protein, 7 aa, Artificial Sequence
  Description of Artificial Sequence Synthetic peptide
NTLKTPR

SEQ ID NO: 395 - protein, 7 aa, Artificial Sequence
  Description of Artificial Sequence Synthetic peptide
YPTLTPR

SEQ ID NO: 396 - protein, 7 aa, Artificial Sequence
  Description of Artificial Sequence Synthetic peptide
SPLKAPR

SEQ ID NO: 397 - protein, 7 aa, Artificial Sequence
  Description of Artificial Sequence Synthetic peptide
HYPYTPR

SEQ ID NO: 398 - protein, 7 aa, Artificial Sequence
  Description of Artificial Sequence Synthetic peptide
SMWFTPR

SEQ ID NO: 399 - protein, 7 aa, Artificial Sequence
  Description of Artificial Sequence Synthetic peptide
SEIRTPR

SEQ ID NO: 400 - protein, 7 aa, Artificial Sequence
  Description of Artificial Sequence Synthetic peptide
KVMQTPR

SEQ ID NO: 401 - protein, 7 aa, Artificial Sequence
  Description of Artificial Sequence Synthetic peptide
SPPFTPR

SEQ ID NO: 402 - protein, 7 aa, Artificial Sequence
  Description of Artificial Sequence Synthetic peptide
SGKLPLL

SEQ ID NO: 403 - protein, 7 aa, Artificial Sequence
  Description of Artificial Sequence Synthetic peptide
FGKLHPA

SEQ ID NO: 404 - protein, 7 aa, Artificial Sequence
  Description of Artificial Sequence Synthetic peptide
LGKLHPL

SEQ ID NO: 405 - protein, 7 aa, Artificial Sequence
  Description of Artificial Sequence Synthetic peptide
SHSKLVA

SEQ ID NO: 406 - protein, 7 aa, Artificial Sequence
  Description of Artificial Sequence Synthetic peptide
SGKLPFV

SEQ ID NO: 407 - protein, 12 aa, Artificial Sequence
  Description of Artificial Sequence Synthetic peptide
TSITELWIINSQ

SEQ ID NO: 408 - protein, 12 aa, Artificial Sequence
  Description of Artificial Sequence Synthetic peptide
TKQTELNMLRLH

SEQ ID NO: 409 - protein, 12 aa, Artificial Sequence
  Description of Artificial Sequence Synthetic peptide
SVKDEVDITLLW

SEQ ID NO: 410 - protein, 12 aa, Artificial Sequence
  Description of Artificial Sequence Synthetic peptide
SQEGDIFQLWNY

SEQ ID NO: 411 - protein, 12 aa, Artificial Sequence
  Description of Artificial Sequence Synthetic peptide
SSFDIFQLWGLR

SEQ ID NO: 412 - protein, 12 aa, Artificial Sequence
  Description of Artificial Sequence Synthetic peptide
SAVQHSDIVHLW

SEQ ID NO: 413 - protein, 7 aa, Artificial Sequence
  Description of Artificial Sequence Synthetic peptide
YDIFTIW

SEQ ID NO: 414 - protein, 7 aa, Artificial Sequence
  Description of Artificial Sequence Synthetic peptide
DDILELW

SEQ ID NO: 415 - protein, 7 aa, Artificial Sequence
  Description of Artificial Sequence Synthetic peptide
TRIFRLW

SEQ ID NO: 416 - protein, 12 aa, Artificial Sequence
  Description of Artificial Sequence Synthetic peptide
EPDHVGRIWDKS

SEQ ID NO: 417 - protein, 7 aa, Artificial Sequence
  Description of Artificial Sequence Synthetic peptide
ADIFSIW

SEQ ID NO: 418 - protein, 7 aa, Artificial Sequence
  Description of Artificial Sequence Synthetic peptide
WDITALW

SEQ ID NO: 419 - protein, 7 aa, Artificial Sequence
  Description of Artificial Sequence Synthetic peptide
TTILDLW

SEQ ID NO: 420 - protein, 7 aa, Artificial Sequence
  Description of Artificial Sequence Synthetic peptide
STIIELW

SEQ ID NO: 421 - protein, 12 aa, Artificial Sequence
  Description of Artificial Sequence Synthetic peptide
GWHTDVLSLWTN

SEQ ID NO: 422 - protein, 7 aa, Artificial Sequence
  Description of Artificial Sequence Synthetic peptide
ALQTVTL

SEQ ID NO: 423 - protein, 7 aa, Artificial Sequence
  Description of Artificial Sequence Synthetic peptide
MWRADLY

SEQ ID NO: 424 - protein, 12 aa, Artificial Sequence
  Description of Artificial Sequence Synthetic peptide
SDFQNYTSRTRH

SEQ ID NO: 425 - protein, 7 aa, Artificial Sequence
  Description of Artificial Sequence Synthetic peptide
ALYIPVL

SEQ ID NO: 426 - protein, 7 aa, Artificial Sequence
  Description of Artificial Sequence Synthetic peptide
LYIPVLG

SEQ ID NO: 427 - protein, 7 aa, Artificial Sequence
  Description of Artificial Sequence Synthetic peptide
SLYIPVL

SEQ ID NO: 428 - protein, 7 aa, Artificial Sequence
  Description of Artificial Sequence Synthetic peptide
NLYIPVL

SEQ ID NO: 429 - protein, 7 aa, Artificial Sequence
  Description of Artificial Sequence Synthetic peptide
LYIPIIP

SEQ ID NO: 430 - protein, 12 aa, Artificial Sequence
  Description of Artificial Sequence Synthetic peptide
TQTLYIPILGPY

SEQ ID NO: 431 - protein, 12 aa, Artificial Sequence
  Description of Artificial Sequence Synthetic peptide
TYLYIPTITQQA

SEQ ID NO: 432 - protein, 12 aa, Artificial Sequence
  Description of Artificial Sequence Synthetic peptide
TYIPVLAPSSET

SEQ ID NO: 433 - protein, 7 aa, Artificial Sequence
  Description of Artificial Sequence Synthetic peptide
AVYIPVL

SEQ ID NO: 434 - protein, 7 aa, Artificial Sequence
  Description of Artificial Sequence Synthetic peptide
TTYIPVL

SEQ ID NO: 435 - protein, 7 aa, Artificial Sequence
  Description of Artificial Sequence Synthetic peptide
TYIPVLV

SEQ ID NO: 436 - protein, 7 aa, Artificial Sequence
  Description of Artificial Sequence Synthetic peptide
YTYIPIL

SEQ ID NO: 437 - protein, 7 aa, Artificial Sequence
  Description of Artificial Sequence Synthetic peptide
YIYIPVL

SEQ ID NO: 438 - protein, 7 aa, Artificial Sequence
  Description of Artificial Sequence Synthetic peptide
ATYISVL

SEQ ID NO: 439 - protein, 7 aa, Artificial Sequence
  Description of Artificial Sequence Synthetic peptide
AHYIPVL

SEQ ID NO: 440 - protein, 7 aa, Artificial Sequence
  Description of Artificial Sequence Synthetic peptide
GMYIPVL

SEQ ID NO: 441 - protein, 7 aa, Artificial Sequence
  Description of Artificial Sequence Synthetic peptide
YTYIPIL

SEQ ID NO: 442 - protein, 7 aa, Artificial Sequence
  Description of Artificial Sequence Synthetic peptide
TYIPILL

SEQ ID NO: 443 - protein, 12 aa, Artificial Sequence
  Description of Artificial Sequence Synthetic peptide
GTYISVLRETVA

SEQ ID NO: 444 - protein, 12 aa, Artificial Sequence
  Description of Artificial Sequence Synthetic peptide
SNATYIPVLQSL

SEQ ID NO: 445 - protein, 12 aa, Artificial Sequence
  Description of Artificial Sequence Synthetic peptide
TYIPVLGMHLNT

SEQ ID NO: 446 - protein, 12 aa, Artificial Sequence
  Description of Artificial Sequence Synthetic peptide
SEIYIPVLSPRA

SEQ ID NO: 447 - protein, 12 aa, Artificial Sequence
  Description of Artificial Sequence Synthetic peptide
STTYISVLNSLG

SEQ ID NO: 448 - protein, 12 aa, Artificial Sequence
  Description of Artificial Sequence Synthetic peptide
QSHHPTYISVLW

SEQ ID NO: 449 - protein, 12 aa, Artificial Sequence
  Description of Artificial Sequence Synthetic peptide
HTHVYISVLGSG

SEQ ID NO: 450 - protein, 12 aa, Artificial Sequence
  Description of Artificial Sequence Synthetic peptide
TITYIPTMPTLT

SEQ ID NO: 451 - protein, 12 aa, Artificial Sequence
  Description of Artificial Sequence Synthetic peptide
SYIYIPTLMNRD

SEQ ID NO: 452 - protein, 12 aa, Artificial Sequence
  Description of Artificial Sequence Synthetic peptide
DWTTYIPTLPTS

SEQ ID NO: 453 - protein, 12 aa, Artificial Sequence
  Description of Artificial Sequence Synthetic peptide
SSTYIPLLRPWI

SEQ ID NO: 454 - protein, 12 aa, Artificial Sequence
  Description of Artificial Sequence Synthetic peptide
NHLYIPVIPGGY

SEQ ID NO: 455 - protein, 12 aa, Artificial Sequence
  Description of Artificial Sequence Synthetic peptide
DPVYNSTIPWMD

SEQ ID NO: 456 - protein, 12 aa, Artificial Sequence
  Description of Artificial Sequence Synthetic peptide
TDAHLYIATIPQ

SEQ ID NO: 457 - protein, 7 aa, Artificial Sequence
  Description of Artificial Sequence Synthetic peptide
MWPLRPP

SEQ ID NO: 458 - protein, 12 aa, Artificial Sequence
  Description of Artificial Sequence Synthetic peptide
SDLNEDLFILWS

SEQ ID NO: 459 - protein, 12 aa, Artificial Sequence
  Description of Artificial Sequence Synthetic peptide
EDLFSLWNHPHQ

SEQ ID NO: 460 - protein, 12 aa, Artificial Sequence
  Description of Artificial Sequence Synthetic peptide
DMNLEDIFWLWR

SEQ ID NO: 461 - protein, 12 aa, Artificial Sequence
  Description of Artificial Sequence Synthetic peptide
NENIFELWASLP

SEQ ID NO: 462 - protein, 12 aa, Artificial Sequence
  Description of Artificial Sequence Synthetic peptide
MQNHEDVVMLWQ

SEQ ID NO: 463 - protein, 12 aa, Artificial Sequence
  Description of Artificial Sequence Synthetic peptide
DWDLTHMTTAMF

SEQ ID NO: 464 - protein, 12 aa, Artificial Sequence
  Description of Artificial Sequence Synthetic peptide
AVEWDLMHPRPF

SEQ ID NO: 465 - protein, 12 aa, Artificial Sequence
  Description of Artificial Sequence Synthetic peptide
TLLWPSNYLILN

SEQ ID NO: 466 - protein, 7 aa, Artificial Sequence
  Description of Artificial Sequence Synthetic peptide
EGIIELW

SEQ ID NO: 467 - protein, 7 aa, Artificial Sequence
  Description of Artificial Sequence Synthetic peptide
EDIISLW

SEQ ID NO: 468 - protein, 7 aa, Artificial Sequence
  Description of Artificial Sequence Synthetic peptide
LWPLWRS

SEQ ID NO: 469 - protein, 7 aa, Artificial Sequence
  Description of Artificial Sequence Synthetic peptide
DWRSVIY

SEQ ID NO: 470 - protein, 7 aa, Artificial Sequence
  Description of Artificial Sequence Synthetic peptide
MSFENIL

SEQ ID NO: 471 - protein, 7 aa, Artificial Sequence
  Description of Artificial Sequence Synthetic peptide
KVVTSMS

SEQ ID NO: 472 - protein, 7 aa, Artificial Sequence
  Description of Artificial Sequence Synthetic peptide
ESLIELW

SEQ ID NO: 473 - protein, 7 aa, Artificial Sequence
  Description of Artificial Sequence Synthetic peptide
EWSLWHA

SEQ ID NO: 474 - protein, 7 aa, Artificial Sequence
  Description of Artificial Sequence Synthetic peptide
YVPWWPA

SEQ ID NO: 475 - protein, 12 aa, Artificial Sequence
  Description of Artificial Sequence Synthetic peptide
EFGPWGFRLTTP

SEQ ID NO: 476 - protein, 12 aa, Artificial Sequence
  Description of Artificial Sequence Synthetic peptide
QHPFTSYIGRFH

SEQ ID NO: 477 - protein, 12 aa, Artificial Sequence
  Description of Artificial Sequence Synthetic peptide
LPYDHLQFSFLD

SEQ ID NO: 478 - protein, 7 aa, Artificial Sequence
  Description of Artificial Sequence Synthetic peptide
SWNPFLP

SEQ ID NO: 479 - protein, 7 aa, Artificial Sequence
  Description of Artificial Sequence Synthetic peptide
YNLSLTF

SEQ ID NO: 480 - protein, 7 aa, Artificial Sequence
  Description of Artificial Sequence Synthetic peptide
WQKQMTI

SEQ ID NO: 481 - protein, 12 aa, Artificial Sequence
  Description of Artificial Sequence Synthetic peptide
TTKTSSLGLWSS

SEQ ID NO: 482 - protein, 12 aa, Artificial Sequence
  Description of Artificial Sequence Synthetic peptide
VEHKSWSLGSLS

SEQ ID NO: 483 - protein, 12 aa, Artificial Sequence
  Description of Artificial Sequence Synthetic peptide
WTKEYSVGLLSS

SEQ ID NO: 484 - protein, 12 aa, Artificial Sequence
  Description of Artificial Sequence Synthetic peptide
YKDYTMGHLSQP

SEQ ID NO: 485 - protein, 12 aa, Artificial Sequence
  Description of Artificial Sequence Synthetic peptide
DWRTSYMERLHG

SEQ ID NO: 486 - protein, 7 aa, Artificial Sequence
  Description of Artificial Sequence Synthetic peptide
NSKDWTV

SEQ ID NO: 487 - protein, 7 aa, Artificial Sequence
  Description of Artificial Sequence Synthetic peptide
LDSKDWS

SEQ ID NO: 488 - protein, 7 aa, Artificial Sequence
  Description of Artificial Sequence Synthetic peptide
TNSKDWT

SEQ ID NO: 489 - protein, 7 aa, Artificial Sequence
  Description of Artificial Sequence Synthetic peptide
LNPKDWT

SEQ ID NO: 490 - protein, 7 aa, Artificial Sequence
  Description of Artificial Sequence Synthetic peptide
WDSKDWS

SEQ ID NO: 491 - protein, 7 aa, Artificial Sequence
  Description of Artificial Sequence Synthetic peptide
KDWTLRL

SEQ ID NO: 492 - protein, 7 aa, Artificial Sequence
  Description of Artificial Sequence Synthetic peptide
KDWTLYN

SEQ ID NO: 493 - protein, 7 aa, Artificial Sequence
  Description of Artificial Sequence Synthetic peptide
GDFKDWT

SEQ ID NO: 494 - protein, 12 aa, Artificial Sequence
  Description of Artificial Sequence Synthetic peptide
LPSYEPFTTRFY

SEQ ID NO: 495 - protein, 7 aa, Artificial Sequence
  Description of Artificial Sequence Synthetic peptide
YEIDIIL

SEQ ID NO: 496 - protein, 12 aa, Artificial Sequence
  Description of Artificial Sequence Synthetic peptide
HYSGFSAWGNRP

SEQ ID NO: 497 - protein, 12 aa, Artificial Sequence
  Description of Artificial Sequence Synthetic peptide
FGPWGNHMPSLL

SEQ ID NO: 498 - protein, 12 aa, Artificial Sequence
  Description of Artificial Sequence Synthetic peptide
FGPWGMQTPLQY

SEQ ID NO: 499 - protein, 7 aa, Artificial Sequence
  Description of Artificial Sequence Synthetic peptide
FGPWGNR

SEQ ID NO: 500 - protein, 7 aa, Artificial Sequence
  Description of Artificial Sequence Synthetic peptide
ASFGPWG

SEQ ID NO: 501 - protein, 7 aa, Artificial Sequence
  Description of Artificial Sequence Synthetic peptide
AFGFGPW

SEQ ID NO: 502 - protein, 7 aa, Artificial Sequence
  Description of Artificial Sequence Synthetic peptide
YMSLPWT

SEQ ID NO: 503 - protein, 7 aa, Artificial Sequence
  Description of Artificial Sequence Synthetic peptide
DYYLPWT

SEQ ID NO: 504 - protein, 7 aa, Artificial Sequence
  Description of Artificial Sequence Synthetic peptide
YSELPWT

SEQ ID NO: 505 - protein, 7 aa, Artificial Sequence
  Description of Artificial Sequence Synthetic peptide
YRTMPWT

SEQ ID NO: 506 - protein, 7 aa, Artificial Sequence
  Description of Artificial Sequence Synthetic peptide
YPSLPFT

SEQ ID NO: 507 - protein, 7 aa, Artificial Sequence
  Description of Artificial Sequence Synthetic peptide
YRTLPWT

SEQ ID NO: 508 - protein, 7 aa, Artificial Sequence
  Description of Artificial Sequence Synthetic peptide
IWSLPWT

SEQ ID NO: 509 - protein, 7 aa, Artificial Sequence
  Description of Artificial Sequence Synthetic peptide
HWNLPFT

SEQ ID NO: 510 - protein, 7 aa, Artificial Sequence
  Description of Artificial Sequence Synthetic peptide
HPTLPFT